SEQUENCE LISTING (14 sequences)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - protein, 188 aa, Homo sapiens [sequence omitted]

SEQ ID NO: 3 - protein, 189 aa, Homo sapiens [sequence omitted]

SEQ ID NO: 4 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 5 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 6 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 7 - protein, 403 aa, Homo sapiens [sequence omitted]

SEQ ID NO: 8 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 9 - protein, 480 aa, Homo sapiens [sequence omitted]

SEQ ID NO: 10 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 11 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 12 - protein, 384 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 13 - protein, 384 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMIKGGVLFPGTDHIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 14 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMIKGGVLFPGTDHIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

(19) United States
(12) Patent Application Publication
(10) Pub. No.: US 2016/0022757 A1
(43) Pub. Date: Jan. 28, 2016

(54) THERAPEUTIC METHODS FOR TREATING SOLID TUMORS AND RELATED DIAGNOSTIC METHODS

(71) Applicants: Erdem BANGI, New York, NY (US); Ross CAGAN, New York, NY (US)

(72) Inventors: Erdem BANGI, New York, NY (US); Ross CAGAN, New York, NY (US)

(73) Assignee: ICAHN SCHOOL OF MEDICINE AT MOUNT SINAI, New York, NY (US)

(21) Appl. No.: 14/775,472

(22) PCT Filed: Mar. 14, 2014
(86) PCT No.: PCT/US14/29445

§ 371 Date: Sep. 11, 2015

Related U.S. Application Data
(60) Provisional application No. 61/799,595, filed on Mar. 15, 2013.
(60) Provisional application No. 61/895,265, filed on Oct. 24, 2013.

(51) Int. Cl. A61K 38/05; A61K 31/519; G01N 33/574; A61K 31/416; A61K 31/7088; A61K 45/06; A61K 31/4745; A61K 31/5377

(52) U.S. Cl. CPC A61K 38/05; A61K 31/4745; A61K 31/519; A61K 31/5377; A61K 31/416; A61K 31/7088; A61K 45/06; G01N 33/57419; G01N 2800/52

(57) ABSTRACT

The present invention provides a method for treating a subject afflicted with a cancer which comprises administering to the subject (i) a proteasome antagonist and (ii) a PI3K signal transduction pathway antagonist, each of (i) and (ii) in an amount such that when both (i) and (ii) are administered, the administration is effective to treat the subject. The present invention also provides a method for treating a subject afflicted with a cancer which comprises administering to the subject (i) a proteasome antagonist, and (ii) an oligonucleotide which decreases the amount of PI3K, mTor, TORC1, TORC2, AKT, or JNK produced by cells of the cancer. The present invention provides processes for identifying whether a compound is an epithelial cancer drug candidate. The present invention also provides a method for identifying a cancer patient who will likely benefit from treatment with a PI3K signal transduction pathway antagonist.

This application claims priority of U.S. Provisional Patent Application No. 61/799,595, filed Mar. 15, 2013 and U.S. Provisional Patent Application No. 61/895,265, filed Oct. 24, 2013, the entire contents of each of which are hereby incorporated herein by reference.

This application incorporates-by-reference nucleotide and/or amino acid sequences which are present in the file named “140314_{—}0028_{—}84569.A PCT_SequenceListing_REB.txt”, which is 64.5 kilobytes in size, and which was created Mar. 14, 2014 in the IBM-PC machine format, having an operating system compatibility with MS-Windows, which is contained in the text file filed Mar. 14, 2014 as part of this application.

Throughout this application, various publications are referenced by Arabic numerals. Pull citations for these publications may be found at the end of the specification immediately preceding the claims. The disclosures of these publications in their entireties are hereby incorporated by reference into this application in order to more fully describe the state of the art to which this invention pertains.

This invention was made with government support under grant numbers R01-CA109730 and R01-CA084309 awarded by the National Institutes of Health. The U.S. Government has certain rights in the invention.

### BACKGROUND OF THE INVENTION

The success rate of cancer clinical trials remains among the lowest of the major diseases (1). One difficulty is that cancer is a multigenic and highly heterogeneous disease: individual tumors typically carry mutations in dozens of genes. Recent genome-wide mutation profiling of human tumors has provided important insights into this genetic complexity and diversity (2). The results have highlighted the difficulties of developing effective therapeutics through models that focus on a single target, and likely explain the low success rate of drugs that have entered clinical trials based on current animal models: approval rates in colon cancer are approximately 3% (3). The discrepancy between preclinical data and clinical trials has been partially attributed to the inadequacy of currently available preclinical animal models (1,4). Use of more complex preclinical models that reflect the multigenic and heterogeneous nature of human tumors will be crucial to bridge this gap. A central challenge, then, is to develop models that reflect tumors' genetic complexities while preserving the detailed interactions between tumor and host. There is a need for such models.

Additionally, there is a need for new treatments and diagnostic methods for cancer.

### SUMMARY OF THE INVENTION

The present invention provides a method for treating a subject afflicted with a cancer which comprises administering to the subject (i) a proteasome antagonist and (ii) a PI3K signal transduction pathway antagonist, each of (i) and (ii) in an amount such that when both (i) and (ii) are administered, the administration is effective to treat the subject.

The present invention provides a method for treating a subject afflicted with a cancer which comprises administering to the subject (i) a proteasome antagonist, and (ii) an oligonucleotide which decreases the amount of PI3K, mTor, TORC1, TORC2, ART, or JNK produced by cells of the cancer, each of (i) and (ii) in an amount that when both (i) and (ii) are administered, the administration is effective to treat the subject.

The present invention provides a pharmaceutical composition comprising (i) a proteasome antagonist and (ii) a PI3K signal transduction pathway antagonist or an oligonucleotide which decreases the amount of PI3K, mTor, TORC1, TORC2, Akt, or JNK produced by cells of the cancer, for use in treating a subject afflicted with a cancer.

The present invention provides a composition for treating a subject afflicted with a cancer comprising (i) a proteasome antagonist and (ii) a PI3K signal transduction pathway antagonist or an oligonucleotide which decreases the amount of PI31, mTor, TORC1, TORC2, AKT, or JNK produced by cells of the cancer.

The present invention provides a process for identifying whether a compound is an epithelial cancer drug candidate comprising
i) obtaining a D. melanogaster which is genetically modified to have
   a) increased Ras activity,
   b) increased PI3K activity, and/or
   c) a reduced level of p53, PTEN, or APC expression or activity,
   in the colon epithelium thereof, such that there is a cancer phenotype in the colon epithelium of the D. melanogaster;
ii) contacting the D. melanogaster with the compound;
iii) determining whether there is a difference between the cancer phenotype of the D. melanogaster of ii) and the cancer phenotype of a corresponding D. melanogaster not contacted with the compound; and
vi) identifying the compound as an epithelial cancer drug candidate if there is a difference between the cancer phenotype of the D. melanogaster contacted with the compound and the cancer phenotype of the corresponding D. melanogaster not contacted with the compound.

The present invention provides a process for identifying whether a combination of a first compound and a second compound is likely to be useful for the treatment of an epithelial cancer comprising
i) obtaining a D. melanogaster which is genetically modified to have
   a) increased Ras activity.
   b) increased PI3K activity, and/or
   c) a reduced level of p53, PTEN, or APC expression or activity,
   in the colon epithelium thereof, such that there is a cancer phenotype in the colon epithelium of the D. melanogaster;
ii) contacting the D. melanogaster with each of the first compound and the second compound;
iii) determining whether there is a difference between the cancer phenotype of the D. melanogaster of ii) and the cancer phenotype of a corresponding D. melanogaster not contacted with the first compound and the second compound; and
   vi) identifying the combination of the first compound and the second compound as likely to be useful for the treatment of epithelial cancer if there is a difference between the cancer phenotype of the at least one D. melanogaster contacted with the first compound and the second compound and the cancer phenotype of the corresponding D. melanogaaster not contacted with the first compound and the second compound.

The present invention provides a process of producing a cancer drug comprising steps i) to iv), followed by
v) producing the compound identified in step iv), thereby producing the cancer drug.

The present invention provides a process for identifying whether a compound is an epithelial cancer drug candidate comprising
i) obtaining an epithelial cell which has
   a) increased Ras activity.
   b) increased PI3K activity, and/or
   c) a reduced level of p53. PTEN, or APC expression or activity,
   such that the epithelial cell has a cancer phenotype;
ii) contacting the epithelial cell with the compound;
iii) determining whether there is a difference between the cancer phenotype of the epithelial cell of ii) and the cancer phenotype of a corresponding epithelial cell not contacted with the compound; and
vi) identifying the compound as an epithelial cancer drug candidate if there is a difference between the cancer phenotype of the epithelial cell contacted with the compound and the cancer phenotype of the corresponding epithelial cell not contacted with the compound.

The present invention provides a process for identifying whether a combination of a first compound and a second compound is likely to be useful for the treatment of an epithelial cancer comprising
i) obtaining an epithelial cell which has
   a) increased Ras activity,
   b) increased PI3K activity, and/or
   c) a reduced level of p53, PTEN, or APC expression or activity.
   such that the epithelial cell has a cancer phenotype;
ii) contacting the epithelial cell with each of the first compound and the second compound;
iii) determining whether there is a difference between the cancer phenotype of the epithelial cell of ii) and the cancer phenotype of a corresponding epithelial cell not contacted with the first compound and the second compound; and
vi) identifying the combination of the first compound and the second compound as likely to be useful for the treatment of epithelial cancer if there is a difference between the cancer phenotype of the at least one epithelial cell contacted with the first compound and the second compound and the cancer phenotype of the corresponding epithelial cell not contacted with the first compound and the second compound.

The present invention provides a process of producing a cancer drug comprising steps i) to iv), followed by
v) producing the compound identified in step iv), thereby producing the cancer drug.

The present invention provides a method for identifying a cancer patient who will likely benefit from treatment with a PI3K signal transduction pathway antagonist comprising
i) obtaining a biological sample comprising cancer tissue from the cancer patient;
ii) detecting whether the cancer tissue in the biological sample
   a) has increased Ras activity and
      α) increased PI3K activity, or
      β) reduced PTEN expression or activity, or
   b) has an increased amount of pAkt and a reduced level of TORC1 activity,
compared to normal tissue of the same type; and
iii) identifying the cancer patient as a cancer patient who will likely benefit from treatment with a PI3K signal tranaduction pathway antagonist if in step (ii) neither
   a) increased Ras activity and
      α) increased PI3K activity, or
      ρ) reduced PTEN expression or activity, nor
   b) an increased amount of pAkt and a reduced level of TORC1 activity,
   is detected in cancer tissue in the biological sample, and identifying the cancer patient as a cancer patient who will not likely benefit from treatment with a PI3K signal transduction pathway antagonist if in step (ii) either
   a) increased Ras activity and
      α) increased PI3K activity, or
      β) reduced PTEN expression or activity, or
   b) an increased amount of pAkt and a reduced level of TORC1 activity,
   is detected in cancer tissue in the biological sample.

The present invention provides a method of treating a cancer patient identified to not likely benefit from treatment with a PI3K signal transduction pathway antagonist comprising the method of the invention.

The present invention provides a kit for identifying a cancer patient who will likely benefit from treatment with a PI3K signal transduction pathway antagonist comprising
i) at least one probe or primer for determining
   a) whether PTEN expression is reduced; or
   b) whether there is a mutation in PTEN reduces the activity thereof,
   in a biological sample, or from nucleic acid obtained from a biological sample, and/or
ii) at least one probe or primer for determining whether there is a mutation in Ras that increases the activity thereof,
   in a biological sample, or from nucleic acid obtained from the biological sample, and/or
iii) at least one probe or primer for determining whether there is a mutation in PI3K that increases the activity thereof.
   in a biological sample, or from nucleic acid obtained from the biological sample, and/or
iv) at least one antibody for determining the amount of p-4EBP in a biological sample, or in protein obtained from the biological sample, and/or
v) at least one antibody for determining the amount of pAKT in a biological sample, or in protein obtained from the biological sample.

### BRIEF DESCRIPTION OF THE FIGURES

FIG. 1. Targeting multigenic colorectal cancer combinations to the adult Drosophila hindgut. a, Most frequently deregulated pathways in colon tumors and the transgenes that represent them. b, Mutation status of individual tumors with respect to the five deregulated pathways. Blue boxes indicate mutation in a pathway component. c, Quadruple mutation combinations in individual human tumors and corresponding combinations generated in Drosophila. d, The adult Drosophila digestive track. Hindgut cells are marked with byn>GFP; nuclei are in red. e-l, control (byn>GFP,dcr2) and ras^{G12v} p53^{RNAi} pten^{RNAi} apc^{RNAi} hindguts 7 and 21 days after induction as labeled. Asterisks (f,h) indicate regions of multilayering. Longitudinal optical sections (i,j) and pylorus regions (k,l) are shown. Abbreviations: crop (cr), midgut (m), malphigian tubules (mp), hindgut (h), rectum (r), pylorus (p), and ileum (i).

FIG. 2. Migration phenotypes induced by quadruple combinations. a-f, control (a) and ras^{G12V} p53R^{NAi} pten^{RNAi} apc^{RNAi} (b-f) ilea; arrows indicate migrating cells. c, Close-up view of b. d-f, Apical-to-basal confocal sections of a migrating cell (asterisk) g,h, Surface views of ras^{G12V} p53^{RNAi} smad4^{RNAi} apc^{RNAi} hindguts with cells migrating on top of muscle layer. i-j, phospho-Src staining of control and ras^{G12V} p53^{RNAi} pten^{RNAi} apc^{RNAi} hindguts. k,l MMP1 staining of control and ras^{G12V} p53^{RNAi} pten^{RNAi} apc^{RNAi} hindguts. k′,l′, MMP1 channel only. m-p, Cross sections of control and ras^{G12V} p53^{RNAi} pten^{RNAi} apc^{RNAi} hindguts. m′-p′, Laminin channel only; arrows indicate reduced/absent Laminin staining. q-w, Dissemination phenotype of quadruple combinations. Arrows indicate GFP-positive foci inside the abdominal cavity (q), below the abdomen epidermis (r), ovaries (s), head (t) and legs (u,v) (t: trachea, n: nephrocyte, f: fat body). w, live confocal image of a multicellular GFP foci inside the abdominal cavity. Nuclei are visualized by a nuclear dsRed transgene (nls-dsRed). x, Quantification of dissemination into the abdominal cavity. none: no dissemination; weak: 1-3 GFP-positive foci inside the abdominal cavity; moderate: 4-10 GFP-positive foci; strong: >10 GFP-positive foci (n=20-30 flies/replicate; error bars reflect standard error of the mean).

FIG. 3. Follow-up analysis of proliferation, multilayering and distant migration phenotypes. a-e, Seven day continuous BRDU labeling (red) of hindguts with the indicated genotypes. Whole hindgut (a) or pylorus regions (b-e) are outlined with solid lines; dashed lines indicate hindgut/midgut boundary (m: midgut). f-J, Whole hindguts of indicated genotypes; asterisks indicate regions of multilayering. k, Quantification of dissemination one week after induction (n=25-30 flies/replicate; error bars: standard error of the mean; *: p<0.01). l,m, top views of ras^{G12V} (1) and ras^{G12V} p53^{RNAi} pten^{RNAi} apc^{RNAi} (m) hindguts with migrating cells on top. Cleaved caspase-3 (n-r) and SA-β-gal (s-v) staining of hindguts with indicated genotypes. Hindguts outlined by solid lines in n-r. w, Features of cancer recapitulated by our multigenic models. x, Summary of interactions between individual transgenes for each phenotype.

FIG. 4. Combinatorial therapy as an effective means to overcome resistance to single agent therapy observed in multigenic models. a,b, Quantification of dissemination in ras^{G12V} (a) and ras^{G12V} p53^{RNAi} pten^{RNAi} apc^{RNAi} (b) animals treated with indicated compounds. c, Summary of compound effects against ras^{G12V} and ras^{G12V} p53^{RNAi} pten^{RNAi} apC^{RNAi}. d, Quantification of dissemination in ras^{G12V} pten^{RNAi} and ras^{G12V} p53^{RNAi} apc^{RNAi} animals treated with BEZ235 e, Western blot analysis of hindguts with indicated genotypes seven days after the induction of transgenes. Syn (Syntaxin): loading control. f, Time course analysis of PI3K pathway activation status in control, ras^{G12V}, pten^{RNAi} and ras^{G12V} p53^{RNAi} pten^{RNAi} apc^{RNAi} hindguts. Each data point represents the average of 2-5 western blots. Error bars represent standard error of the mean. a. g,h, Quantification of dissemination in ras^{G12V} p53^{RNAi} pten^{RNAi} apc^{RNAi} (g) and ras^{G12V} pten^{RNAi} (h) animals treated with indicated compounds. i, Western blot analysis of ras^{G12V} p53^{RNAi} pten^{RNAi} apc^{RNAi} hindguts seven days after treatment with indicated compounds. j, Schematic illustration of the mechanism of resistance to BEZ235 and LY294002 and the mechanism by which combinatorial therapy overcomes resistance. k, Quantification of dissemination in ras^{G12V} p53^{RNAi} pten^{RNAi} apc^{RNAi} animals after sequential treatment with bortezomib and BEZ235 as indicated. (a,b,d,g,h,k: n=30 flies/replicate; error bars: standard error of the mean *: p<0.01; **: p<0.05).

FIG. 5. Validation of BEZ resistance and effectiveness of combinatorial therapy in human colorectal cancer line DLD-1. a, BEZ235 dose response curve of DLD-1 parental (Ras and PI3K active) versus DLD-1 WT (Ras active, PI3K wildtype) cell lines. b, PI3K pathway activation status of DLD-1 cells after 4 hour treatment with the indicated doses of bortezomib. c,d, Time course of PI3K pathway activation by indicated doses of bortezomib in DLD-1 cells after 1, 4, 12, 18 and 24 hours of treatment. Each data point represents the average of 2-3 western blots. Error bars represent standard error of the mean. e, BEZ235 dose response curve of DLD-1 cells after pretreatment with DMSO (control) or indicated doses of bortezomib for 24 hours.

FIG. 6. Multigenic combinations generated in Drosophila and corresponding human tumors. Tumor IDs shown in bold are exact matches to their corresponding combinations with respect to mutated genes. Others match to their corresponding tumors with respect to the deregulated pathways but not the mutated genes.

FIG. 7. Survival curves of multigenic combinations after induction of transgenes. Survival curves of quadruple phenotypes (a), and of subcombinations that make up the 4 hit combination ras^{G12V} p53^{RNAi} pten^{RNAi} apc^{RNAi} (b).

FIG. 8. Follow-up analysis of cancer phenotypes observed in ras^{G12V} p53^{RNAi} pten^{RNAi} apc^{RNAi}. a-d, 7 day continuous BRDU labeling (red) of hindguts with the indicated genotypes. Pylorus region of the hindguts are outlined with solid lines; dashed lines indicate hindgut/midgut boundary (m: midgut). e-j, Cleaved caspase-3 staining of hindguts (outlined by solid lines) with indicated genotypes. Hindgut cells occasionally displayed high levels of membrane-associated cleaved caspase (e.g. f), though its functional significance is unclear. k, Quantification of distant migration in animals with indicated genotypes 1, 2, 3 and 4 weeks after induction (n=25-30 flies/replicate; error bars: standard error of the mean). l,m, Examples of GFP negative cells from hindguts carrying ras^{G12V} (l) and ras^{G12V} pten^{RNAi} (m). n, SA-β-gal positive enterocytes in hindguts carrying ras^{G12V} were also GFP negative (n′). o-s, SA-β-gal staining of hindguts with indicated genotypes.

FIG. 9. MAPK pathway activation status in ras^{G12V} and ras^{G12V} p53^{RNAi} pten^{RNAi} apc^{RNAi} hindguts. Western blot analysis of MAPK activity as measured by dual ERK phosphorylation (dpERK) in in ras^{G12V} and ras^{G12V} p53^{RNAi} pten^{RNAi} apc^{RNAi} hindguts 7 days after the induction of transgenes.

FIG. 10. Comparison of migrating cell sizes in subcombinations of ras^{G12V} and ras^{G12V} p53^{RNAi} pten^{RNAi} apc^{RNAi}. Examples of cells migrating on top of hindguts with indicated genotypes.

FIG. 11. Targets, feeding doses and toxicity of compounds. a, List of compounds used for feeding experiments along with their targets, mechanisms of actions and concentrations used in the food. Estimated amount ingested was calculated based on our observations that adult females ingest approximately 0.2 μl of food per day (not shown). Estimated amount of ingested compound was also converted into mg/kg body weight/day using 1.1 mg as the average weight of an adult female. b, Survival curves of ras^{G12V} p53^{RNAi} pten^{RNAi} apc^{RNAi} animals fed the indicated compounds. At the doses used in our experiments, compounds did not have significant toxicity.

FIG. 12. Toxicity and efficacy of LBH589 and Bortezomib. a, Survival curves of ras^{G12V} p53^{RNAi} pten^{RNAi} apc^{RNAi} animals fed LBH589 or Bortezomib at indicated doses, b,c, Quantification of distant migration phenotype of animals with indicated genotypes after feeding different doses of bortezomib (b) and LBH589 (c). n=30 flies/replicate; error bars: standard error of the mean *: p<0.01; **: p<0.05

FIG. 13. PI3K pathway activity in subcombinations of ras^{G12V} p53^{RNAi} pten^{RNAi} apc^{RNAi}. Western blot analysis of PI3K pathway activity in hindguts from indicated genotypes as measured by AKT and 4EBP phosphorylation seven days after the induction of transgenes.

FIG. 14. Combinatorial treatment of remaining compounds with Bortezomib. Quantification of distant migration phenotype of ras^{G12V} p53^{RNAi} pten^{RNAi} apc^{RNAi} animals after feeding indicated compounds in combination with bortezomib. n=30 flies/replicate; error bars: standard error of the mean *: p<0.01; **: p<0.05

FIG. 15. Increased sensitivity of DLD-1 cells to BEZ235 after pretreatment with bortezomib. a, BEZ235 dose response curve after 3 days of BEZ235 treatment of DLD-1 cells that are pretreated with DMSO or indicated doses of bortezomib for 1 day. b, Bortezomib pre-treatment alone does not affect the viability of DLD-1 cells.

FIG. 16. Validation of BEZ resistance and effectiveness of combinatorial therapy in human colorectal cancer line HCT116. a, BEZ235 dose response curve of HCT116 parental (Ras and PI3K active) versus HCT116-WT (Ras active, PI3K wildtype) cell lines. b, Bortezomib pre-treatment alone does not affect the viability of HCT116 cells. c,d, BEZ235 dose response curve after 2 (c) and 3 (d) days of treatment of HCT116 cells that are pretreated with DMSO (control) or indicated doses of bortezomib for 24 hours.

FIG. 17. Tumors with coactivation of Ras/MAPK and PI3K pathways are resistant to PI3K pathway inhibitors. a, Dissemination of tumor cells from the Drosophila colon into the abdominal cavity used as a quantitative read-out to monitor drug response. Phenotypic categories are determined by the number of disseminated foci in the abdomical cavity in each animal. b,c, Dissemination induced by ras^{G12V} alone is sensitive to PI3K pathway inhibitors (b) whereas dissemination phenotype observed in the four-hit model ras^{G12V} p53^{RNAi} pten^{RNAi} apc^{RNAi} is resistant (a). d, Summary of compound effects against ras^{G12V} and ras^{G12V} p53^{RNAi} pten^{RNAi} apc^{RNAi}. e, Quantification of dissemination in ras^{G12V} pten^{RNAi} and ras^{G12V} p53^{RNAi} apc^{RNAi} animals treated with BEZ235, indicating that resistance to PI3K pathway inhibitors observed in the four-hit model is mediated by loss of pten. (b,c,e: n=30 flies/replicate; error bars: standard error of the mean *: p<0.01; **: p<0.05).

FIG. 18. Resistance to PI3K inhibitors correlates with chronic high phospho-AKT and low TORC1 activity in response to co-activation of Ras/MAPK and PI3K pathways in Drosophila. a, Western blot analysis of Drosophila colons with indicated genotypes seven days after the induction of transgenes. Syn (Syntaxin): loading control. b, Quantification of the western blot data shown in a. Each data point represents the average of 2-5 western blots. Error bars represent standard error of the mean.

FIG. 19. Low-dose Bortezomib treatment overcomes resistance to PI3K pathway inhibitors in Ras and PI3K activated tumors. a,b, Quantification of dissemination phenotype in ras^{G12V} p53^{RNAi} pten^{RNAi} apc^{RNAi} (a) and ras^{G12V} pten^{RNAi} animals (b) treated with Bortezomib and PI3K pathway inhibitors. c, Quantification of dissemination in ras^{G12V} p53^{RNAi} pten^{RNAi} apc^{RNAi} animals after sequential treatment with bortezomib and BEZ235 as indicated. (a,b,c % n=30 flies/replicate; error bars: standard error of the mean *: p<0.01; **: p<0.05).

FIG. 20. Pretreatment with Bortezomib renders Ras and PI3K active colorectal cancer cell lines more sensitive to PI3K pathway inhibitors. a, BEZ235 dose response curve of HCT116 (a) cells after pretreatment with DMSO (control) or indicated doses of bortezomib for 24 hours. b, Bortezomib pretreatment alone has no effect on viability of DLD-1 and HCT116 cells at doses that sensitize the cells to BEZ235.

FIG. 21. Tumor growth during the course of treatment.

FIG. 22. Tumor growth during the course of treatment.

FIG. 23. Tumor growth at the end of treatment.

FIG. 24. Tumor growth at the end of treatment.

### DETAILED DESCRIPTION OF THE INVENTION

The present invention provides a method for treating a subject afflicted with a cancer which comprises administering to the subject (i) a proteasome antagonist and (ii) a PI3K signal transduction pathway antagonist, each of (i) and (ii) in an amount such that when both (i) and (ii) are administered, the administration is effective to treat the subject.

In some embodiments, the subject is a mammal.

In some embodiments, the mammal is human.

In some embodiments, the cancer is in the form of a solid tumor.

In some embodiments, cancer is colon cancer.

In some embodiments, the colon cancer is resistant to treatment.

In some embodiments, the colon cancer is resistant to treatment with a PI3K signal transduction pathway antagonist.

In some embodiments, the PI3K signal transduction pathway antagonist is an organic compound having a molecular weight less than 1000 Daltons, a DNA aptamer, an RNA aptamer, or a polypeptide, which antagonist binds to PI3K, mTor, TORC1, TORC2, AKT, or JNK.

In some embodiments, the PI3K signal transduction pathway antagonist is a DNA aptamer, an RNA aptamer, or a polypeptide.

In some embodiments, the PI3K signal transduction pathway antagonist is an organic compound having a molecular weight less than 1000 Daltons.

In some embodiments, the organic compound has the structure:

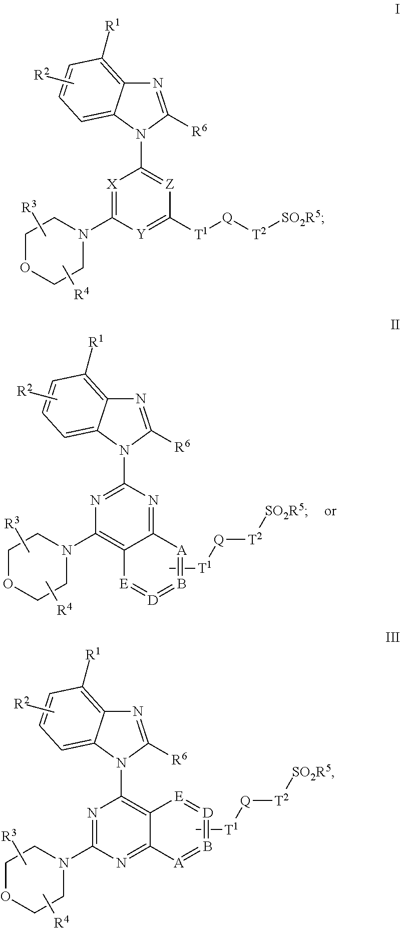

or an enantiomer, a mixture of enantiomers, or a mixture of two or more diastereomers of any of I, II, or III; or a pharmaceutically acceptable salt, solvate, hydrate, or prodrug form of any of I, II, or III;
wherein:
   each R¹ and R² is independently (a) hydrogen, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1b}, —C(O)NR^{1b}R^{1c}, —C(NR^{a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiments, one two three or four, substituents Q¹; or (iii) R^{1b} and R^{1c} together with the N atom to which they are attached form heterocyclyl, optionally substituted with one or more, in one embodiment, one two three or four, substituents Q¹;
   each R³ and R⁴ is independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
   each R⁵ is independently C₁₋₆ alkyl, C₂₋₆ alkenyl, C₂₋₆ alkynyl, C₃₋₇ cycloalkyl, C₃₋₇ cycloalkyl, C₆₋₁₄ aryl, C₇₋₁₅ aralkyl, heteroaryl, or heterocyclyl;
   each R⁶ is independently hydrogen or C₁₋₆ alkyl;
   each A, B, D, and E is independently (i) a bond; (ii) a nitrogen, oxygen, or sulfur atom; or (iii) CR⁷, where R⁷ is hydrogen, halo, C₁₋₆ alkyl, C₂₋₆ alkenyl, or C₂₋₆ alkynyl; wherein the bonds between A, B, D, and E may be saturated or unsaturated; with the proviso that no more than one of A, B, D, and E are a bond;
   each Q is C₁₋₆ alkylene, C₂₋₆ alkenylene, C₂₋₆ alkynylene, C₃₋₇ cycloalkylene, C₆₋₁₄ arylene, heteroarylene, or heterocyclylene;
   each T¹ is independently a bond, —O—, or —NR⁸—;
   each T² is independently a bond or —NR⁸—, with the proviso that the atom that is attached to —SO₂R⁵ is nitrogen;
   each R⁸ is independently hydrogen, Ca₁₋₆ alkyl, C₂₋₆ alkenyl, or C₂₋₆ alkynyl; and
   X, Y, and Z are each independently a nitrogen atom or CR⁹, with the proviso that at least two of X, Y, and Z are nitrogen atoms; where R⁹ is hydrogen or C₁₋₆ alkyl;
   wherein each alkyl, alkylene, alkenyl, alkenylene, alkynyl, alkynylene, cycloalkyl, cycloalkylene, aryl, arylene, heteroaryl, heteroarylene, heterocyclyl, and heterocyclylene is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O) R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}—, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;
   wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)N^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}; R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In some embodiments, the organic compound has the structure:

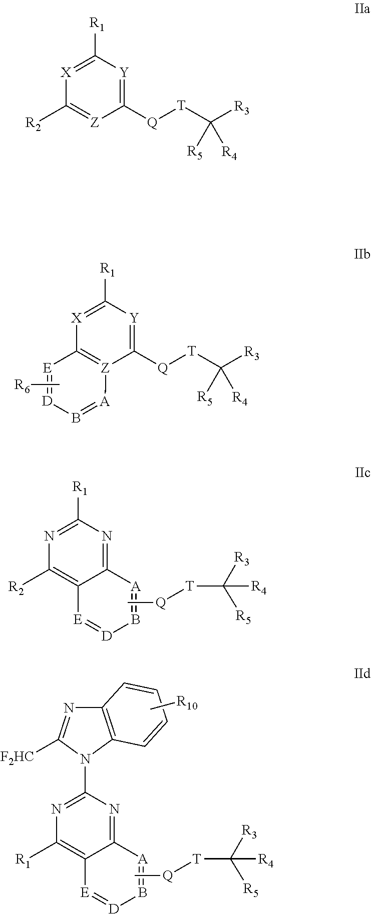

or an enantiomer, a mixture of enantiomers, or a mixture of two or more diastereomers of any of IIa to IId; or a pharmaceutically acceptable salt, solvate, hydrate, or prodrug of any of IIa to IId; wherein:
   each R₁ is independently C₆₋₁₄ aryl, heteroaryl, or heterocyclyl;
   each R₂ is independently C₆₋₁₄ aryl, heteroaryl, or heterocyclyl;
   each R₃ and R₄ is independently hydrogen, lower alkyl, C₂₋₆ alkenyl, C₂₋₆ alkynyl, or R₅;
   each R₅ is independently halogen or —OSO₂R₇;
   R₆ is C₃₋₇ cycloalkyl, C₆₋₁₄ aryl, heteroaryl, or heterocyclyl;
   R₇ is lower alkyl, C₂₋₆ alkenyl, C₂₋₆ alkynyl, C₃₋₇ cycloalkyl, C₆₋₁₄ aryl, heteroaryl, or heterocyclyl;
   R₁₀ is (a) hydrogen, amino, or hydroxyl; or (b) lower alkyl, lower alkylamino, di(lower alkyl)amino, lower alkoxy, or carboxamido;
   each Q is independently absent or a linker group;
   each T is independently —CO—, —CS—, or —SO₂—;
   X, Y, and Z are each independently a nitrogen atom or CR₈, with the proviso that at least two of X, Y, and Z are nitrogen atoms; wherein R₈ is hydrogen or lower alkyl; and
   each A, B, D, and E is independently (i) a direct bond; (ii) a nitrogen, oxygen, or sulfur atom; or (iii) CR₉, where R₉ is hydrogen, halogen, or lower alkyl; wherein the bonds between A, B, D, and E may be saturated or unsaturated; with the proviso that no more than one of A, B, D, and E are a direct bond;
   wherein each alkyl, alkenyl, alkynyl, alkoxy, alkylamino, dialkylamino, carboxamido, cycloalkyl, aryl, heteroaryl, and heterocyclyl is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;
   wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}; R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In some embodiments, the organic compound has the structure:

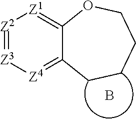

or
a stereoisomer, geometric isomer, tautomer, or pharmaceutically acceptable salt thereof, wherein:
B is a pyrazolyl, imidazolyl, or triazolyl ring fused to the benzoxepin ring and selected from the structures:

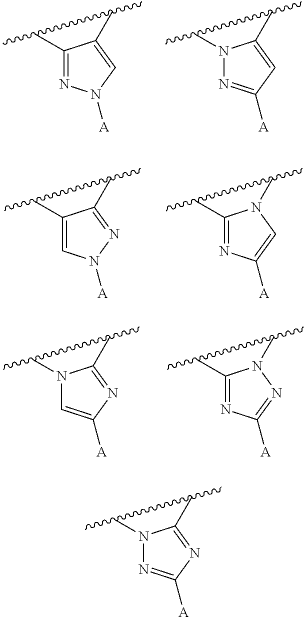

Z¹ is CR¹ or N;
Z² is CR² or N;
Z³ is CR³ or N;
Z⁴ is CR⁴ or N;
R¹, R², R³, and R⁴ are independently selected from H, F, Cl, Br, I, —CN, —COR¹⁰, —CO₂R¹⁰, —C(═)N(R¹⁰)OR¹¹, —C(═NR¹⁰)NR¹⁰R¹¹, —C(═O)NR¹⁰R¹¹, —NO₂, —NR¹⁰R¹¹, —NR¹²C(═O)R¹⁰, —NR¹²C(═O)OR¹¹, —NR¹²C(═O)NR¹⁰R¹¹, —NR¹²C(═O)(C₁-C₁₂alkylene)NR¹⁰R¹¹, NR¹² (C₁-C₁₂ alkylene)NR¹⁰R¹¹, —NR¹² (C₁-C₁₂alkylene)OR¹⁰, —NR¹²(C₁-C₁₂ alkylene)C(═O)NR¹⁰R¹¹, —OR¹⁰, —SR¹⁰, —S(O)₂R¹⁰,
—C(═O)NR¹⁰(C₁-C₁₂ alkylene)NR¹⁰R¹¹,
—C(═O)NR¹⁰(C₁-C₁₂ alkylene)NR¹⁰C(═O)OR¹¹,
—C(═O)NR¹⁰(C₁-C₁₂ alkylene)NR¹⁰C(═O)R¹¹,
—C(═O)NR¹⁰(C₁-C₁₂ alkylene)R¹⁰,
C₁-C₁₂ alkyl,
C₂-C₈ alkenyl,
C₂-C₈ alkynyl,
C₃-C₁₂ carbocyclyl,
C₂-C₂₀ heterocyclyl,
C₆-C₂₀ aryl,
C₁-C₂₀ heteroaryl,
—(C₃-C₁₂ carbocyclyl)-(C₁-C₁₂alkyl),
—(C₂-C₂₀ heterocyclyl)-(C₁-C₁₂ alkyl),
—(C₆-C₂₀ aryl)-(C₁-C₁₂ alkyl),
—(C₁-C₂₀ heteroaryl)-(C₁-C₁₂ alkyl),
—(C₁-C₁₂ alkylene)-(C₃-C₁₂ carbocyclyl),
—(C₁-C₁₂ alkylene)-(C₂-C₂₀ heterocyclyl),
—(C₁-C₁₂ alkylene)-(C₂-C₂₀ heterocyclyl)-(C₂-C₂₀ heterocyclyl),
—(C₁-C₁₂ alkylene)-(C₂-C₂₀ heterocyclyl)-(C₃-C₁₂ carbocyclyl),
—(C₁-C₁₂ alkylene)-(C₂-C₂₀ heterocyclyl)-C(═O)—(C₂-C₂₀ heterocyclyl),
—(C₁-C₂₀ alkylene)-(C₁-C₂₀ heteroaryl),
—(C₁-C₁₂ alkylene)-(C₂-C₂₀ heterocyclyl)-(C₁-C₁₂ alkyl),
—(C₁-C₁₂ alkylene)-(C₆-C₂₀ aryl)-(C₁-C₁₂ alkyl),
—(C₁-C₁₂ alkylene)-(C₁-C₂₀ heteroaryl)-(C₁-C₁₂ alkyl),
—(C₁-C₁₂ alkylene)-C(═O)—(C₂-C₂₀ heterocyclyl),
—(C₁-C₁₂ alkylene)C(═O)OR¹⁰,
—(C₁-C₁₂ alkylene)C(═O)NR¹⁰R¹¹,
—(C₁-C₁₂ alkylene)-NR¹⁰R¹¹,
—(C₁-C₁₂ alkylene)NRC(═O)R¹⁰,
—(C₁-C₁₂ alkylene)OR¹⁰,
—(C₁-C₁₂ alkylene)-NR¹⁰—(C₁-C₁₂ alkylene)-(C₁-C₂₀ heteroaryl),
—(C₁-C₁₂ alkylene)-NR¹⁰—(C₁-C₁₂ alkylene)-(C₁-C₂₀ heterocyclyl),
—(C₁-C₁₂ alkylene)-NR¹⁰—(C₁-C₁₂ alkylene)-NHC(═O)—(C₁-C₂₀heteroaryl),
—(C₁-C₁₂ alkylene)-(C₂-C₂₀ heterocyclyl)-NR¹⁰R¹¹, and
—(C₁-C₁₂ alkylene)-(C₂-C₂₀ heterocyclyl)-(C₁-C₁₂ alkyl)-NR¹⁰R¹¹,
where alkyl, alkenyl, alkynyl, alkylene, carbocyclyl, heterocyclyl, aryl, and heteroaryl are optionally substituted with one or more groups independently selected from F, Cl, Br, I, R¹⁰, —SR¹⁰, —S(O)₂R¹⁰, —S(O)₂NR¹⁰R¹¹, NR¹⁰R¹¹, —NR¹²C(O)R¹⁰, CO₂R¹⁰, —C(O)R¹⁰, —CONR¹⁰R¹¹, oxo, and —OR¹⁰;
A is selected from —C(═O)NR⁵R⁶, —NR⁵R⁶, C₆-C₂₀ aryl, C₂-C₂₀heterocyclyl and C₁-C₂₀ heteroaryl wherein aryl, heterocyclyl and heteroaryl are optionally substituted with one or more groups independently selected from F, Cl, Br, I, —CN, —COR¹⁰, —CO₂R¹⁰, —C(═O)N(R¹⁰)OR¹¹, —C(═NR¹⁰)NR¹⁰R¹¹, —C(═O)NR¹⁰R¹¹, —NO₂, —NR¹⁰R¹¹, —NR¹²C(═O)R¹⁰, —NR¹²C(═O)OR¹¹, —NR¹²C(═O)NR¹⁰R¹¹, —NR¹²C(═O)(C₁-C₁₂ alkylene)NR¹⁰R¹¹, —NR¹²(C₁-C₁₂ alkylene)NR¹⁰R¹¹, —NR¹²(C₁-C₁₂ alkylene)OR¹⁰, —NR¹²(C₁-C₁₂ alkylene)C(═O)NR¹⁰R¹¹, —OR¹⁰, —S(O)₂R¹⁰,
—C(═O)NR¹⁰(C₁-C₁₂ alkylene)NR¹⁰R¹¹,
—C(═O)NR¹⁰(C₁-C₁₂ alkylene)NR¹⁰C(═O)OR¹¹,
—C(═O)NR¹⁰(C₁-C₁₂ alkylene)NR¹⁰C(═O)R¹¹,
—C(═O)NR¹⁰(C₁-C₁₂ alkylene)R¹⁰,
C₁-C₁₂ alkyl,
C₂-C₈ alkenyl,
C₂-C₈ alkynyl,
C₃-C₁₂ carbocyclyl,
C₂-C₂₀ heterocyclyl,
C₆-C₂₀ aryl,
C₁-C₂₀ heteroaryl,
—(C₃-C₁₂ carbocyclyl)-(C₁-C₁₂ alkyl),
—(C₂-C₂₀ heterocyclyl)-(C₁-C₁₂ alkyl),
—(C₆-C₂₀ aryl)-(C₁-C₁₂ alkyl),
—(C₁-C₂₀ heteroaryl)-(C₁-C₁₂ alkyl),
—(C₁-C₁₂ alkylene)-(C₃-C₁₂ carbocyclyl),
—(C₁-C₁₂ alkylene)-(C₂-C₂₀ heterocyclyl),
—(C₁-C₁₂ alkylene)-(C₂-C₂₀ heterocyclyl-(C₂-C₂₀ heterocyclyl),
—(C₁-C₁₂ alkylene)-(C₂-C₂₀ heterocyclyl)-(C₃-C₁₂ carbocyclyl),
—(C₁-C₁₂ alkylene)-(C₂-C₂₀ heterocyclyl)-C(═O)—(C₂-C₂₀ heterocyclyl),
—(C₁-C₁₂ alkylene)-(C₁-C₂₀ heteroaryl),
—(C₁-C₁₂ alkylene)-(C₂-C₂₀ heterocyclyl)-(C₁-C₁₂ alkyl),
—(C₁-C₁₂ alkylene)-(C₆-C₂₀ aryl)-(C₁-C₁₂ alkyl),
—(C₁-C₁₂ alkylene)-(C₁-C₂₀ heteroaryl)-(C₁-C₁₂ alkyl),
—(C₁-C₁₂ alkylene)-C(═O)—(C₂-C₂₀ heterocyclyl),
—(C₁-C₁₂ alkylene)C(═O)OR¹⁰,
—(C₁-C₁₂ alkylene)-NR¹⁰R¹¹,
(C₁-C₁₂ alkylene)NR¹²C(═O)R¹⁰,
—(C₁-C₁₂ alkylene)OR¹⁰,
—(C₁-C₁₂ alkylene)-NR¹⁰—(C₁-C₁₂ alkylene)-(C₁-C₂₀ heteroaryl),
—(C₁-C₁₂ alkylene)-NR¹⁰—(C₁-C₁₂ alkylene)-(C₁-C₂₀ heterocyclyl),
—(C₁-C₁₂ alkylene)-NR¹⁰—(C₁-C₁₂ alkylene)-NHC(═O)—(C₁-C₂₀heteroaryl),
—(C₁-C₁₂ alkylene)-(C₂-C₂₀ heterocyclyl)-NR¹⁰R¹¹, and
—(C₁-C₁₂ alkylene)-(C₂-C₂₀ heterocyclyl)-(C₁-C₁₂ alkyl)-NR¹⁰R¹¹,
where alkyl, alkenyl, alkynyl, alkylene, carbocyclyl, heterocyclyl, aryl, and heteroaryl are optionally substituted with one or more groups independently selected from F, Cl, Br, I, R¹⁰, —SR¹⁰, —S(O)₂R¹⁰, NR¹⁰R¹¹, —NR¹²C(O)R¹⁰, —CO₂R¹⁰, —CONR¹⁰R¹¹, and —OR¹⁰;
R⁵ is selected from H, and C₁-C₁₂ alkyl, optionally substituted with one or more groups independently selected from F, Cl, Br, I, —CN, —CO₂H, —CONH₂, —CONHCH₃, —NH₂, —NO₂, —N(CH₃)₂, —NHCOCH₃, —NHS(O)₂CH₃, —OH, —OCH₃, —OCH₂CH₃, —S(O)₂NH₂, and —S(O)₂CH₃;
R⁶ is selected from C₁-C₂ alkyl, C₃-C₁₂ carbocyclyl, C₂-C₂₀ heterocyclyl, C₁-C₂₀ heteroaryl, and C₆-C₂₀ aryl, each optionally substituted with one or more groups independently selected from F, Cl, Br, I, —CH₃, —CH₂OH, —CH₂C₆H₅, —CN, —CF₃, —CO₂H, —C(O)CH₃, —NH₂, —NO₂, —N(CH₃)₂, —NHCOCH₃, —NHS(O)₂CH₃, —OH, oxo, —OCH₃, —OCH₂CH₃, —S(O)₂NH₂, —S(O)₂CH₃, —C(═O)NR¹⁰(C₁-C₁₂ alkylene)NR¹⁰R¹¹, phenyl, pyridinyl, tetrahydro-furan-2-yl, 2,3-dihydro-benzofuran-2-yl, 1-isopropyl-pyrrolidin-3-ylmethyl, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, —C≡CR¹³, —CH═CHR¹³, and —C(═O)NR¹⁰R¹¹;
or R⁵ and R⁶ together with the nitrogen atom to which they are attached form C₂-C₂₀ heterocyclyl or C₁-C₂₀ heteroaryl, optionally substituted with one or more groups selected from F, Cl, Br, I, CH₃, C(CH₃)₃, —CH₂OH, —CH₂CH₂OH, —CH₂C₆H₅, pyridin-2-yl, 6-methyl-pyridin-2-yl, pyridin-4-yl, pyridin-3-yl, pyrimidin-2-yl, pyrazin-2-yl, tetrahydro-furan-carbonyl, 2-methoxy-phenyl, benzoyl, cyclopropylmethyl, (tetrahydrofuran-2-yl)methyl, 2,6-dimethyl-morpholin-4-yl, 4-methyl-piperazine-carbonyl, pyrrolidine-1-carbonyl, cyclopropanecarbonyl, 2,4-difluoro-phenyl, pyridin-2-ylmethyl, morpholin-4-yl, —CN, —CF₃, —CO₂H, —CONH₂, —CONHCH₃, —CON(CH₃)₂, —COCF₃, —COCH₃, —COCH(CH₃)₂, —NO₂, NHCH₃, —N(CH₃)₂, —N(CH₃CH₃)₂, —NHCOCH₃, —NCH₃COCH₃, —NHS(O)₂CH₃, —OH, —OCH₃, —OCH₂CH₃, —CH₂OCH₃, —CH₂CH₂OCH₃, —CH₂S(O)₂NHCH₃, —CH₂S(O)₂CH₂CH₃, —S(O)₂NHCH₃, —S(O)CH₂CHCH, —S(O)₂NH₂, —S(O)₂N(CH₃)₂ and —S(O)₂CH₃;
R¹⁰, R¹¹ and R¹² are independently selected from H, C₁-C₁₂ alkyl, —(C₁-C₁₂ alkylene)-(C₂-C₂₀ heterocyclyl), —(C₁-C₁₂ alkylene)-(C₆-C₂₀ aryl), —(C₁-C₂₀ alkylene)-(C₃-C₁₂ carbocyclyl), C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl, each of which are optionally substituted with one or more groups independently selected from F, Cl, Br, I, —CH₃, —CH₂CH₃, —CH(CH₃)₂, —CH₂OH, —CH₂OCH₃, —CH₂CH₂OH, —C(CH₃)₂OH, —CH₂C(CH₃) OH, —CH₂CH (CH₃) OH, —CH₂CO₂H, —CH₂CO₂CH₃, —CH₂NH₂, —(CH₂)₂N(CH₃)₂, —CH₂C₆H₅, —CN, —CF₃, —CO₂H, —C(O)CH₃, —C(O)CH(OH)CH₃, —CO₂CH₃, —CONH₂, —CONHCH₃, —CON(CH₃)₂, —C(CH₃)₂CONH₂, —NH₂, —NO₂, —N(CH₃)₂, —N(CH₃)C(CH₃)₂CONH₂, —N(CH₃)CH₂CH₂S (O) CH₃, —NHCOCH₃, —NHS(O)₂CH₃, ═O(oxo), —OH, —OCH, —OCH₂CH₃, —OCH₂CH₂OH, —OP(O)(OH)₂, —SCH₃, —S(O)₂CH₃, —S(O)₂NH₂, —S(O)₂N(CH₃)₂, —CH₂S(O)₂NHCH₃, —CH₂S(O)₂CH₂CH₃, —S(O)₂NHCH₃, —S(O)₂CH₂CH₃, pyrrolidin-1-yl, 2-oxopyrrolidin-1-yl, cyclopropyl, cyclopentyl, oxetanyl, 4-methylpiperazin-1-yl, and 4-morpholinyl;
or R¹⁰ and R¹¹ together with the nitrogen atom to which they are attached form a C₂-C₂₀ heterocyclyl ring or C₁-C₂₀ heteroaryl each of which are optionally substituted with one or more groups independently selected from F, Cl, Br, I, —CH₃, —CH₂OH, —CH₂C₆H₅, —CN, —CF₃, —CO₂H, —CONH₂, —CONHCH₃, —NO₂, —N(CH₃)₂, —NHCOCH₃, —NHS(O)₂CH₃, —OH, oxo, —OCH₃, —OCH₂CH₃, —S(O)₂NH₂, —S(O)₂CH₃, —CH(CH₃)₂, —CH₂CF₃, —CH₂CH₂OH and —C(CH₃)₂OH; and
R¹³ is selected from H, F, Cl, Br, I, —CH₃, —CH₂CH₃, —CN, —CF₃, —CH₂N(CH₃)₂, —CH₂OH, —CO₂H, —CONH₂, —CON(CH₃)₂, —NO₂, and —S(O)₂CH₃.

In some embodiments, the organic compound has the structure:

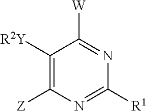

wherein
   R¹ is selected from:
   (i) a group of the following formula:

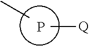

wherein
   P is (i) aryl or heteroaryl which is unsubstituted or substituted;
      (ii) an indazole group which is unsubstituted or substituted;
      (iii) an indole group which is unsubstituted or substituted; or
      (iv) a benzoimidazole group which is unsubstituted or substituted;
   Q is selected from —H, —OR, —SR, -Halo, —NR₃R₄, —OS(O)ₘR, —OC(O)R, —OC(O)NHR, —S(O)ₘNR₃R₄, —NRC(O)R, —NRS(O)ₘR, —NRC(O)NR₃R₄, and —NRC(S)NR₃R₄, wherein each R, R₃, and R₄ is independently selected from H, C₁-C₆ alkyl, C₃-C₁₀ cycloalkyl and a 5- to 12-membered carbocyclic group, aryl or heteroaryl group, the group being unsubstituted or substituted; m is 1 or 2; or R₃ and R₄, which are the same or different, are each independently selected from H, C₁-C₆ alkyl which is unsubstituted or substituted, C₃-C₁₀ cycloalkyl which is unsubstituted or substituted, —C(O)R, —C(O)N(R)₂ and —S(O)ₘR wherein R and m are as defined above, or R₃ and R₄ together with the nitrogen atom to which they are attached form a saturated 5-, 6- or 7-membered N-containing heterocyclic group which is unsubstituted or substituted; —C(O)R, —C(O)N(R)₂ and —S(O)ₘR wherein R and m are as defined above;
   Y is selected from —O—(CH₂)ₙ—, —S—(CH₂)ₙ—, and —S(O)ₘ(CH₂)ₙ— wherein m is 1 or 2, n is 0 or an integer of 1 to 3, and R² is selected from H or a 5- to 12-membered carbocyclic or heterocyclic group which is unsubstituted or substituted, and a group —NR₃R₄ wherein R₃ and R₄ are as defined above;
   Z is selected from (i) halo, —(CH₂)ₛCOOR, —(CH₂)ₛCHO, —(CH₂)ₛCH₂OR, —(CH₂)ₛCONR₃R₄, —(CH₂)ₛCH₂NR₃R₄, —NR₃R₄ and —O(CH₂)ₛNR₃R₄ wherein s is 0 or an integer of 1 to 2 and wherein R, R₃ and R₄ are as defined above; (ii) substituted or unsubstituted heteroaryl, (iii) substituted or unsubstituted heterocyclyl, (iv) substituted or unsubstituted aryl, and (v) substituted or unsubstituted C₁-C₆-alkyl; and
   W is selected from (i) NR₄R₄, wherein R₅ and R₆ form, together with the N atom to which they are attached, a morpholine ring which is unsubstituted or substituted, (ii) substituted or unsubstituted heteroaryl, (iii) substituted or unsubstituted heterocyclyl, (iv) substituted or unsubstituted aryl, and (v) substituted or unsubstituted C₁-C₆-alkyl;
or a stereoisomer, or a tautomer, or an N-oxide, or a pharmaceutically acceptable salt, or an ester, or a prodrug, or a hydrate, or a solvate thereof.

In some embodiments, the organic compound has the structure:

### Va

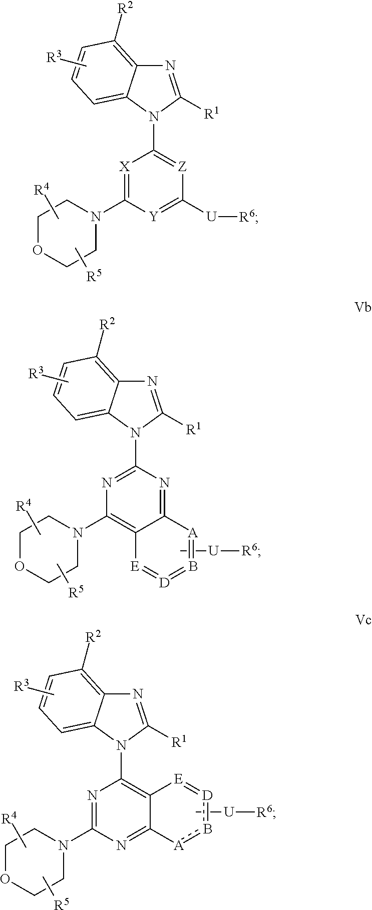 [0249] or
or an enantiomer, a mixture of enantiomers, or a mixture of two or more diastereomers of any of Va, Vb, or Vc; or a pharmaceutically acceptable salt, solvate, hydrate, or prodrug of any of Va, Vb, or Vc; wherein:
   each R¹ is independently hydrogen, C₁₋₆ alkyl, —S—C₁₋₆ alkyl, —S(O)—C₁₋₆ alkyl, or —SO₂—C₁₋₆ alkyl;
   each R² and R³ is independently (a) hydrogen, cyano, halo, ornitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyelyl; or (c) —C(O)R^{1a}, —C(O)OR^{1b}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)₂NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
   each R⁴ and R⁵ is independently hydrogen or C₁₋₆ alkyl; or R⁴ and R⁵ are linked together to form a bond, C₁₋₆ alkylene, C₁₋₆ heteroalkylene, C₂₋₆ alkenylene, or C₂₋₆ heteroalkenylene;
   each R⁶ is independently C₆₋₁₄ aryl, C₇₋₁₅ aralkyl, heteroaryl, or heteroaryl-C₁₋₆ alkyl;
   each U is independently a bond, —C(O)—, —C(O)O—, —C(O)NR^{1a}—, —O—, —OC(O)O—, —OC(O)NR^{1a}—, —NR^{1a}—, —NR^{1a}C(O)NR^{1d}—, —NR^{1a}S(O)—, —NR^{1a}S(O)₂—, —NR^{1a}S(O)NR1d-, —NR^{1a}S(O)₂NR^{1d}—, —S—, —S(O)—, or —S(O)₂—;
   each X, Y, and Z is independently N or CR⁷, with the proviso that at least two of X, Y, and Z are nitrogen atoms; where R⁷ is hydrogen or C₁₋₆ alkyl; and
   each A, B, D, and E is independently a bond, C, O, N, S, NR⁹, CR⁹, or CR⁹R¹⁰, where each R⁹ and R¹⁰ is independently hydrogen, halo, C₁₋₆ alkyl, C₂₋₆ alkenyl, or C₂₋₆ alkynyl; wherein the bonds between A, B, D, and E may be saturated or unsaturated; with the proviso that no more than one of A, B, D, and E are a bond;
   each R^{1a}, R^{1b}, R^{1c}, and R^{1d} is independently (i) hydrogen; or (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;
   wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁵, R⁶, R⁷, R⁹, R¹⁰, R^{1a}, R^{1b}, R^{1c}, or R^{1d} is optionally substituted with one or more, in one embodiment, one, two, three, or four groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyelyl, each of which is further optionally substituted with one or more, in one embodiment, one, two, three, or four, substituents Q; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O) R^{d}, —NR^{a}S(O)NR^{b}R_{c}, —SR^{a}, —S(O)R^{a}, and —S(O)NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q; or (iii) Rb and Rc together with the N atom to which they are attached form heterocyclyl, optionally substituted with one or more, in one embodiment, one, two, three, or four, substituents Q;
   wherein each Q is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}—OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)ORₕ, —NReC(O)NR^{f}R^{g}, —NR^{e}C(═NR^{h})NR^{f}R^{g}, —NR^{e}S(O)R^{h}, —NR^{e}S(O)NR^{f}R^{g}, —SR^{e}, —S(O)R^{e}, and —S(O)NR^{f}R^{g}, wherein each R^{e}, R^{f}; R^{g}, and R^{h} is independently (i) hydrogen; (ii) C₁₋₆ alkyl, C₃₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In some embodiments, the organic compound has the structure:

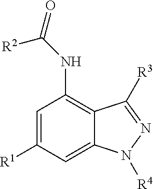

wherein
   R¹ is phenyl substituted by one or two substituents independently selected from C₁₋₆ alkyl, —OR⁵, halo, —CN, —COR⁶, CO₂R⁷, —CONR⁸R⁹, —NR¹⁰R¹¹, —NHCOR¹², —SO₂R¹³, —(CH₂)ₘSO₂NR¹⁴R¹⁵, —NHSO₂R₁₆, and 5-membered heteroaryl wherein the 5-membered heteroaryl contains one or two heteroatoms independently selected from oxygen and nitrogen; or pyridinyl optionally substituted by one or two substituents independently selected from C₁₋₆ alkyl, —OR¹⁷, halo, —SO₂R¹⁸, —SO₂NR¹⁹R²⁰, —NHSO₂R²¹ and —NHCOR²⁴;
   R² is —(CH₂)ₙ-phenyl optionally substituted by —CN or —NR²²R²³; 5- or 6-membered heteroaryl wherein the 5- or 6-membered heteroaryl contains one or two heteroatoms independently selected from oxygen, nitrogen and sulphur and is optionally substituted by C₁₋₆ alkyl, halo or —(CH₂)_{q}NR²⁵R²⁶; or C₃₋₆ cycloalkyl optionally substituted by phenyl;
   R³ is hydrogen or fluoro;
   R⁴ is hydrogen or methyl;
   R⁷, R¹⁷, R¹⁹, R²⁰, R²², R²³, R²⁷, R²⁸ and R²⁹ are each independently hydrogen or C₁₋₆ alkyl;
   R⁵ is hydrogen, C₁₋₆ alkyl or —CF₃;
   R⁶, R¹², R¹³, R¹⁸, R³³ and R³⁴ are each independently C₁₋₆ alkyl;
   R⁸ and R⁹ are each independently hydrogen or C₁₋₆ alkyl, or R⁸ and R⁹, together with the nitrogen atom to which they are attached, are linked to form a 5- or 6-membered heterocyclyl optionally containing an oxygen atom;
   R¹⁰ and R¹¹ are each independently hydrogen or C₁₋₆ alkyl, or R¹⁰ and R¹¹, together with the nitrogen atom to which they are attached, are linked to form a 5- or 6-membered heterocyclyl optionally containing an oxygen atom;
   R¹⁴ and R¹⁵ are each independently hydrogen, C₁₋₆ alkyl, C₃₋₆ cycloalkyl or —(CH₂)ₚphenyl, or R¹⁴ and R¹⁵, together with the nitrogen atom to which they are attached, are linked to form a 5- or 6-membered heterocyclyl optionally containing an oxygen atom;
   R¹⁶ is C₁₋₆ alkyl; or phenyl optionally substituted by C₁₋₆ alkyl;
   R²¹ is C₃₋₆ cycloalkyl; C₁₋₆ alkyl optionally substituted by —CF₃; phenyl optionally substituted by one or two substituents independently selected from C₁₋₆ alkyl, —OR²⁷, —CO2R²⁸ and halo; —(CH₂)ᵤNR³⁵R³⁶; or 5-membered heteroaryl wherein the 5-membered heteroaryl contains one or two heteroatoms independently selected from oxygen, nitrogen and sulphur and is optionally substituted by one or two substituents independently selected from C₁₋₆ alkyl;
   R²⁴ is C₁₋₆ alkyl optionally substituted by —OR²⁹;
   R²⁵ and R²⁶, together with the nitrogen atom to which they are attached, are linked to form a 5-, 6- or 7-membered heterocyclyl or a 10-membered bicyclic heterocyclyl wherein the 5-, 6- or 7-membered heterocyclyl or the 10-membered bicyclic heterocyclyl optionally contains an oxygen atom, a sulphur atom or a further nitrogen atom and is optionally substituted by one or two substituents independently selected from C₁₋₆ alkyl, C₃₋₆ cycloalkyl, halo, oxo, phenyl optionally substituted by halo, pyridinyl, —(CH₂)ᵣR³⁰, —(CH₂)ₛNR³¹R³², —COR³³ and —SO₂R³⁴;
   R³⁰ is hydrogen, C₁₋₆ alkyl or —(CH₂), phenyl;
   R³¹ and R³², together with the nitrogen atom to which they are attached, are linked to form a 6-membered heterocyclyl optionally containing an oxygen atom:
   R³⁵ and R³⁶, together with the nitrogen atom to which they are attached, are linked to form a 5- or 6-membered heterocyclyl wherein the 5- or 6-membered heterocyclyl optionally contains an oxygen atom or a further nitrogen atom and is optionally substituted by one or two substituents independently selected from C₁₋₆ alkyl;
   m, n, p, q, r, s and t are each independently 0, 1 or 2; and u is 1 or 2; or a salt thereof.

In some embodiments, the organic compound has the structure:

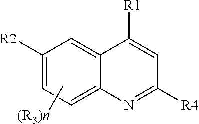

in which
R2 is an optionally substituted ring system selected from a group consisting of: formula (A), (B), (C), (D), (E), (F), (G), (H) and (I):

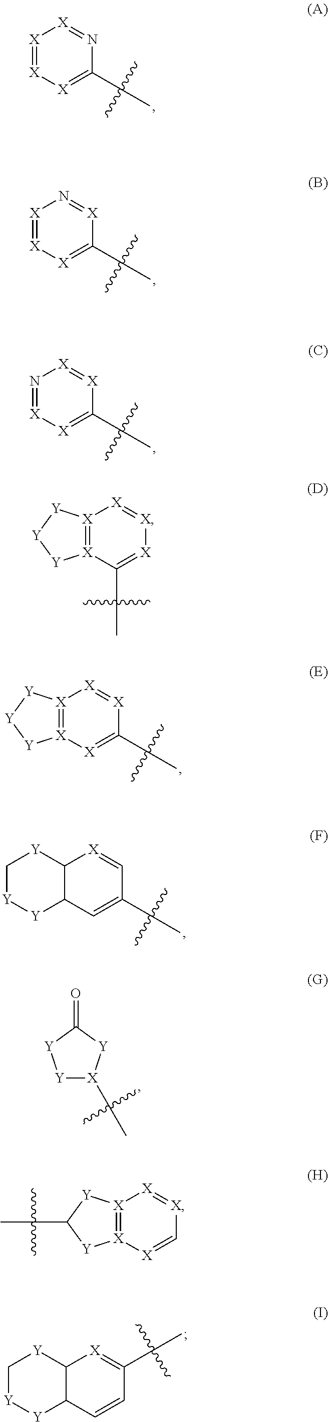

R1 is selected from a group consisting of: heterocycloalkyl, substituted heterocycloalkyl, aryl, substituted aryl, heteroaryl and substituted heteroaryl; each R3 and R4 is independently selected from: hydrogen, halogen, acyl, amino, substituted amino, C₁₋₆ alkyl, substituted C₁₋₆ alkyl, C₃₋₇ cycloalkyl, substituted C₃₋₇ cycloalkyl, C₃₋₇ heterocycloalkyl, substituted C₃₋₇ heterocycloalkyl, alkylcarboxy, aminoalkyl, aryl, substituted aryl, heteroaryl, substitutedheteroaryl, arylalkyl, substituted arylalkyl, arylcycloalkyl, substituted arylcycloalkyl, heteroarylalkyl, substituted heteroarylalkyl, cyano, hydroxyl, alkoxy, nitro, acyloxy, and aryloxy;
n is 1-2;
X is C or N; Y is C, O, N or S;
or a pharmaceutically acceptable salt thereof,
provided that in each of formula (D) to (I) at least one X or Y is not carbon; further provided that R2 is not quinoline or substituted quinoline.

In some embodiments, the organic compound has the structure:

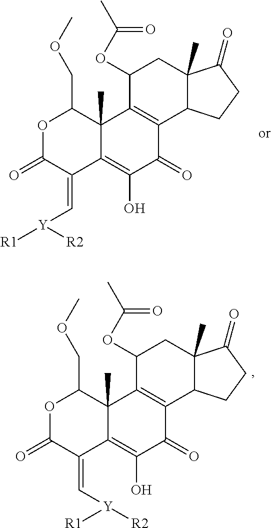

wherein Y is a heteroatom and R1 or R2 are unsaturated alkyl, non-linear alkyl, or substituted alkyl, including a branched alkyl or cyclic alkyl.

In some embodiments, the organic compound has the structure:

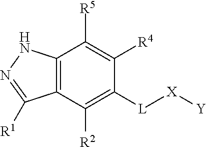

wherein
   R¹ is a C₆-C₁₄ aromatic cyclic hydrocarbon group which may be substituted or a 5- to 14-membered aromatic heterocyclic group which may be substituted;
   R², R⁴ and R⁵ each independently represent a hydrogen atom, a halogen atom, a hydroxyl group, a cyano group, a nitro group, a carboxyl group, a C₁-C₈ alkyl group which may be substituted, a C₁-C₆ alkoxy group which may be substituted, a C₂-C₇ acyl group which may be substituted, —CO—NR^{2a}R^{2b}, —NR^{2b}CO—R^{2a} or —NR^{2a}R^{2b}, wherein R^{2a} and R^{2b} each independently represent a hydrogen atom or a C₁-C₆ alkyl group which may be substituted;
   L is a single bond, a C₁-C₆ alkylene group which may be substituted, a C₂-C₈ alkenylene group which may be substituted or a C₂-C₆ alkynylene group which may be substituted;
   X is a single bond, or a group represented by —NR⁶—, —O—, —CO—, —S—, —SO—, —SO₂—, —CO—NR⁸—V²—, —C(O)O—, —NR⁸—CO—V²—, —NRS—C(O)O—, —NR⁸—S—, —NRS—SO—, —NR⁸—SO₂—V²—, —NR⁸—CO—NR¹⁰—, —NR⁹—CS—NR¹⁰—, —S(O)ₘ—NR¹¹—V²—, —C(═NR¹²)—NR¹³—, —OC(O)—, —OC(O)—N—R¹⁴— or —CH₂—NR⁸—COR⁶ wherein R⁶, R⁸, R⁹, R¹⁰, R¹¹, R¹², R¹³ and R¹⁴ each independently represent a hydrogen atom, a halogen atom, a hydroxyl group, a C₁-C₆ alkyl group which may be substituted, a C₂-C₆ alkenyl group which may be substituted, a C₂-C₆ alkynyl group which may be substituted, a C₁-C₆ alkoxy group which may be substituted, a C₂-C₆ alkenyloxy group which may be substituted, a C₁-C₆ alkylthio group which may be substituted, a C₂-C₆ alkenylthio group which may be substituted, a C₃-C₈ cycloalkyl group which may be substituted, a C₃-C₈ cycloalkenyl group which may be substituted, a 5- to 14-membered non-aromatic heterocyclic group which may be substituted, a C₆-C₁₄ aromatic cyclic hydrocarbon group which may be substituted or a 5- to 14-membered aromatic heterocyclic group which may be substituted; V² is a single bond or a C₁-C₆ alkylene group which may be substituted; and m is 0, 1 or 2; and
   Y is a hydrogen atom, a halogen atom, a nitro group, a hydroxyl group, a cyano group, a carboxyl group, a C₁-C₆ alkyl group which may be substituted, a C₂-C₆ alkenyl group which may be substituted, a C₂-C₆ alkynyl group which may be substituted, a C₁-C₆ alkoxy group which may be substituted, a C₃-C₈ cycloalkyl group which may be substituted, a C₃-C₈ cycloalkenyl group which may be substituted, a 5- to 14-membered non-aromatic heterocyclic group which may be substituted, a C₆-C₁₄ aromatic cyclic hydrocarbon group which may be substituted, a 5- to 14-membered aromatic heterocyclic group which may be substituted, an amino group or —W—R¹⁵, wherein W is —CO— or —SO₂—; and R¹⁵ is a C₁-C₆ alkyl group which may be substituted, a C₆-C₁₄ aromatic cyclic hydrocarbon group which may be substituted, a 5- to 14-membered aromatic heterocyclic group which may be substituted or an amino group,
   or a salt or a hydrate thereof.

In some embodiments, the organic compound has the structure:

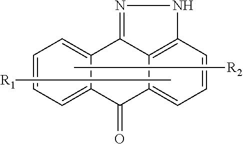

or a pharmaceutically acceptable salt thereof, wherein:
   R₁ and R₂ are optional substituents that are the same or different and independently represent alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, or a group represented by formula (a), (b), (c) or (d):

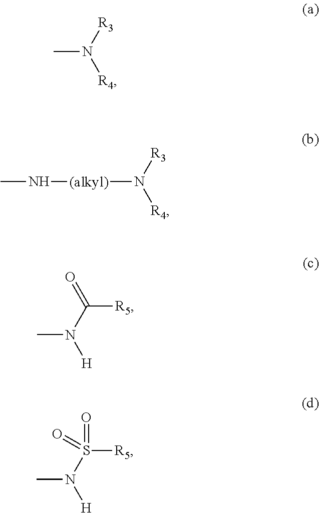

R₃ and R₄ taken together represent alkylidene or a heteroatom-containing alkylidene, or R₃ and R₄ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino); and
   R₅ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino.

In some embodiments, the organic compound has the structure:

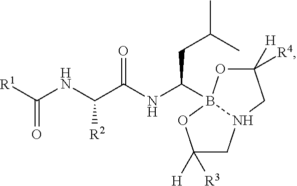

or a boronic ester thereof,
wherein
R¹ is 2-(6-phenyl)pyridinyl, R² is (1R)-1-hydroxyethyl, and R³ and R⁴ are H;
R¹ is 2-(6-phenyl)pyridinyl, R² is (1R)-1-hydroxyethyl, and R³ and R⁴ are methyl; or
R¹ is 2-pyrazinyl, R² is benzyl, and R³ and R⁴ are H.

In some embodiments, the organic compound has the structure:

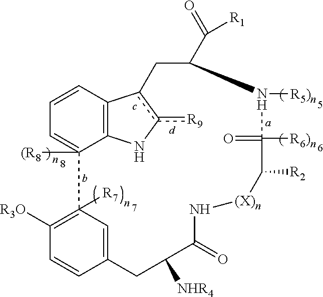

wherein:
   at least one of the bonds a and b, and only one of the bonds c or d, are present, provided that:
      when the bonds a and b are present simultaneously, then R₉ is H, and n₅=n₆ n₇=n₈=0,
      when the bond a is present, but not the bond b, then n₅=n₆=0, and n₇=n₈=1,
      when the bond b is present, but not the bond a, then n₅=n₆=1, and n₇=n₈=0,
      when the bond c is present, and d is absent, then R₉ is H,
      when the bond d is present, and c is absent, then R₉ is an oxygen atom O,
   n₀ is 0 or 1, and when n₀ is 1, X═CH₂ or X═NCH₂C₆H₅,
   R₁ is:
      OH, or a OR₁₀ group in which R₁₀ is a linear or branched alkyl group from 1 to 5 carbon atoms,
   or a group of formula NH—(CH₂)ₙ₁—R₁₁ in which:
   n₁=0, or an integer from 1 to 5,
      R₁₁ is a linear or branched alkyl group from 1 to 5 carbon atoms, an aryl group, possibly substituted, NH₂, or NHR₁₂ in which R₁₂ is a protecting group of amine functions, such as the tertiobutyloxycarbonyl (Boc) group, or the CO—O—CH₂—C₆H₅ (Z) group,
   R₂ is:
      H, or a linear or branched alkyl group from 1 to 5 carbon atoms,
      or a group of formula (CH₂)ₙ₂—(CO)ₙ₃—NR₁₃R₁₄, in which:
         n₂ is an integer from 1 to 5,
         n₃=0 or 1,
      R₁₃ and R₁₄, independently from one another, are:
         H,
         or a protecting group of amine functions, such as Boc, or Z,
         or a group of formula C(═NH)NHR₁₅ in which R₁₅ is H or a protecting group of amine functions, such as Boc, or Z, mentioned above,
   or a side chain from proteogenic amino acids,
   R₃ is H, or a linear or branched alkyl group from 1 to 5 carbon atoms, optionally substituted with an aryl group,
   R₄ is H, or a protecting group of amine functions, such as Boc, or Z,
   R₅ is H, or a protecting group of amine functions, such as Boc, or Z,
   R₆ is a OR₁₆ group in which R₁₆ is a linear or branched alkyl group from 1 to 5 carbon atoms,
   R₇ and R₈, independently from one another, are H, or a halogen atom, such as Br, I, or Cl.

In some embodiments, the PI3K signal transduction pathway antagonist binds to PI3K and has the structure:

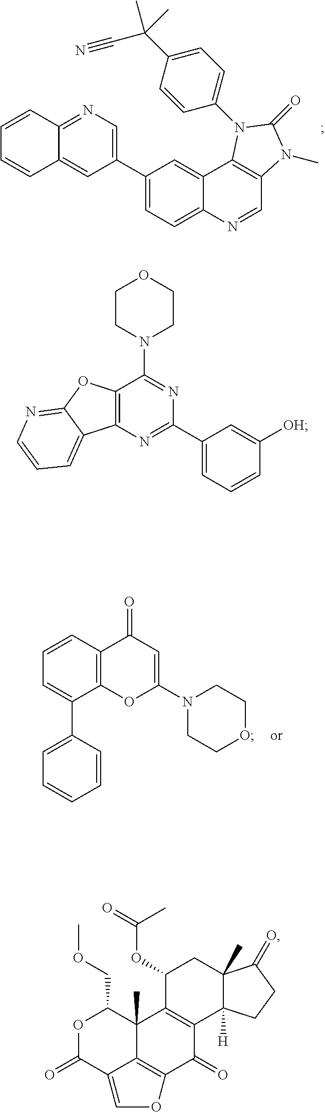 [SMILES: Cn1c(=O)n(-c2ccc(C(C)(C)C#N)cc2)c2c3cc(-c4cnc5ccccc5c4)ccc3ncc21 ; Oc1cccc(-c2nc(N3CCOCC3)c3oc4ncccc4c3n2)c1 ; O=c1cc(N2CCOCC2)oc2c(-c3ccccc3)cccc12 ; COC[C@H]1OC(=O)c2coc3c2[C@@]1(C)C1=C(C3=O)[C@@H]2CCC(=O)[C@@]2(C)C[C@H]1OC(C)=O]

or a pharmaceutically acceptable salt or ester thereof.

In some embodiments, the PI3K signal transduction pathway antagonist is capable of separately binding both PI3K and mTor.

In some embodiments, the PI3K signal transduction pathway antagonist binds to JNK and has the structure:

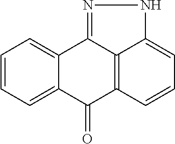 [SMILES: O=C1c2ccccc2-c2n[nH]c3cccc1c23]

or a pharmaceutically acceptable salt or ester thereof.

In some embodiments, the proteasome antagonist is an organic compound having a molecular weight less than 1000 Daltons, a DNA aptamer, an RNA aptamer, or a polypeptide, which antagonist inhibits proteasome function.

In some embodiments, the proteasome antagonist is a DNA aptamer, an RNA aptamer, or a polypeptide.

In some embodiments, the proteasome antagonist is an organic compound having a molecular weight less than 1000 Daltons.

In some embodiments, the proteasome antagonist has the structure:

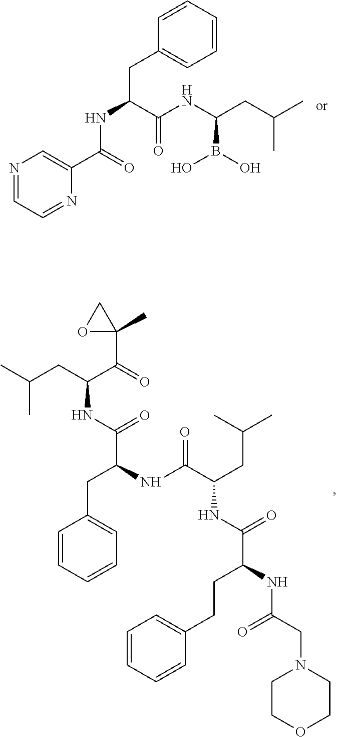

or a pharmaceutically acceptable salt or ester thereof.

In some embodiments, the amount of the proteasome antagonist when administered is effective to increase TORC1 activity in cells of the cancer, as measured by an increase in p-4EBP in the cells of the cancer.

In some embodiments, the proteasome antagonist is administered to the subject before the PI3K signal transduction pathway antagonist, such that the PI3K signal transduction pathway antagonist is administered during at least a portion of the time that the proteasome antagonist is active in the subject.

In some embodiments, the proteasome antagonist is administered to the subject concurrently with the PI3K signal transduction pathway antagonist.

In some embodiments, the amount of pAKT is increased and TORC1 activity is decreased in cells of the cancer compared to cells from tissue of the same type.

In some embodiments, the Receptor Tyrosine Kinase (RTK)/Ras signal transduction pathway and the PI3K signal transduction pathway are misregulated in cells of the cancer compared to cells from tissue of the same type.

In some embodiments, the Ras signal transduction pathway and the PI3K signal transduction pathway each have a higher level of activation in cells of the cancer compared to cells from tissue of the same type.

In some embodiments, Ras and PI3K each have a higher level of activation in cells of the cancer compared to cells from tissue of the same type.

In some embodiments, cells of the cancer have at least one activating mutant allele in Ras.

In some embodiments, the at least one activating mutant allele in Ras is in K-Ras.

In some embodiments, cells of the cancer express a K-Ras mutant protein having a G12X substitution, wherein the numbering of the K-Ras amino acid sequence is relative to the sequence set forth in SEQ ID NO: 2 or 3.

In some embodiments, the K-Ras mutant protein has a G12V substitution.

In some embodiments, the K-Ras mutant protein is a K-Ras^{G12V} mutant protein.

In some embodiments, the K-Ras mutant protein has a G12D substitution.

In some embodiments, the K-Ras mutant protein is a K-Ras^{G12D} mutant protein.

In some embodiments, cells of the cancer express a K-Ras mutant protein having a G13X substitution, wherein the numbering of the K-Ras amino acid sequence is relative to the sequence set forth in SEQ ID NO: 2 or 3.

In some embodiments, cells of the cancer express a K-Ras mutant protein having a Q61X substitution, wherein the numbering of the K-Ras amino acid sequence is relative to the sequence set forth in SEQ ID NO: 2 or 3.

In some embodiments, the at least one activating mutant allele in Ras is in N-Ras.

In some embodiments, cells of the cancer express a N-Ras mutant protein having a G12X substitution, wherein the numbering of the N-Ras amino acid sequence is relative to the sequence set forth in SEQ ID NO: 4.

In some embodiments, the N-Ras mutant protein is a N-Ras^{G12V} or a N-Ras^{G12D} mutant protein.

In some embodiments, cells of the cancer express a N-Ras mutant protein having a G13X substitution, wherein the numbering of the N-Ras amino acid sequence is relative to the sequence set forth in SEQ ID NO: 4.

In some embodiments, cells of the cancer express a N-Ras mutant protein having a Q61X substitution, wherein the numbering of the N-Ras amino acid sequence is relative to the sequence set forth in SEQ ID NO: 4.

In some embodiments, the at least one activating mutant allele in Ras is in H-Ras.

In some embodiments, cells of the cancer express a H-Ras mutant protein having a G12X substitution, wherein the numbering of the H-Ras amino acid sequence is relative to the sequence set forth in SEQ ID NO: 5 or 6.

In some embodiments, the H-Ras mutant protein is a H-Ras^{G12V} or a H-Ras^{G12D} mutant protein.

In some embodiments, cells of the cancer express a H-Ras mutant protein having a G13X substitution, wherein the numbering of the H-Ras amino acid sequence is relative to the sequence set forth in SEQ ID NO: 5 or 6.

In some embodiments, cells of the cancer express a H-Ras mutant protein having a Q61X substitution, wherein the numbering of the H-Ras amino acid sequence is relative to the sequence set forth in SEQ ID NO: 5 or 6.

In some embodiments, cells of the cancer have at least one activating mutant allele in a subunit of PI3K.

In some embodiments, cells of the cancer express a PI3K mutant subunit.

In some embodiments, the PI3K subunit is a p110 catalytic subunit.

In some embodiments, the p110 catalytic subunit is a p110α, β, or δ catalytic subunit.

In some embodiments, the at least one activating mutant allele is in the PIK3CA gene, which encodes the p110α catalytic subunit of PI3K.

In some embodiments, cells of the cancer express a p110α mutant subunit having a E542K, E545K, H1047R, or H1047L substitution, relative to the sequence set forth in SEQ ID NO: 1.

In some embodiments, the p110α mutant subunit has a E542K substitution.

In some embodiments, the p110α mutant subunit has a E545K substitution.

In some embodiments, the p110α mutant subunit has a H1047R substitution.

In some embodiments, the p110α mutant subunit has a H1047L substitution.

In some embodiments, cells of the cancer have reduced PTEN function compared to cells from tissue of the same type.

In some embodiments, cells of the cancer have at least one mutant allele in PTEN that is a deletion mutation, and/or is a mutation that results in the reduced or loss of PTEN protein function in cells of the cancer that express the PTEN mutant protein.

In some embodiments, the at least one mutant allele in PTEN results in a change at amino acid R130, R233, R130, R130, V317, R173, N323, R173, R130, P248, L318, K6, Y76, Q214, R130, E242, I101, G129, E299, or L139 relative to the amino acid sequence of PTEN set forth in SEQ ID NO: 7.

In some embodiments, the mutation reduces the catalytic activity of PTEN compared to PTEN not having the mutation.

In some embodiments, cells of the cancer have a reduced level of PTEN protein expression.

In some embodiments, cells of the cancer have a reduced level of PTEN protein expression and express a Ras^{G12V} mutant protein.

The present invention provides a method for treating a subject afflicted with a cancer which comprises administering to the subject (i) a proteasome antagonist, and (ii) an oligonucleotide which decreases the amount of PI3K, mTor, TORC1, TORC2, AKT, or JNK produced by cells of the cancer, each of (i) and (ii) in an amount that when both (i) and (ii) are administered, the administration is effective to treat the subject.

In some embodiments, the oligonucleotide comprises nucleotides in a sequence that is complementary to PI3K, mTor, AKT, or JNK-encoding mRNA.

In some embodiments, the oligonucleotide is an antisense oligodeoxynucleotide.

In some embodiments, the oligonucleotide is an RNA interference inducing compound.

In some embodiments, the oligonucleotide is a ribozyme.

The present invention provides a pharmaceutical composition comprising (i) a proteasome antagonist and (ii) a PI3K signal transduction pathway antagonist or an oligonucleotide which decreases the amount of PI3K, mTor, TORC1, TORC2, Akt, or JNK produced by cells of the cancer, for use in treating a subject afflicted with a cancer.

The present invention provides a composition for treating a subject afflicted with a cancer comprising (i) a proteasome antagonist and (ii) a PI3K signal transduction pathway antagonist or an oligonucleotide which decreases the amount of PI3K, mTor, TORC1, TORC2, AKT, or JNK produced by cells of the cancer.

The present invention provides a process for identifying whether a compound is an epithelial cancer drug candidate comprising
i) obtaining a D. melanogaster which is genetically modified to have
   a) increased Ras activity,
   b) increased PI3K activity, and/or
   c) a reduced level of p53, PTEN, or APC expression or activity,
   in the colon epithelium thereof, such that there is a cancer phenotype in the colon epithelium of the D. melanogaster;
ii) contacting the D. melanogaster with the compound;
iii) determining whether there is a difference between the cancer phenotype of the D. melanogaster of ii) and the cancer phenotype of a corresponding D. melanogaster not contacted with the compound; and
vi) identifying the compound as an epithelial cancer drug candidate if there is a difference between the cancer phenotype of the D. melanogaster contacted with the compound and the cancer phenotype of the corresponding D. melanogaster not contacted with the compound.

The present invention provides a process for identifying whether a combination of a first compound and a second compound is likely to be useful for the treatment of an epithelial cancer comprising
i) obtaining a D. melanogaster which is genetically modified to have
   a) increased Ras activity,
   b) increased PI3K activity, and/or
   c) a reduced level of p53, PTEN, or APC expression or activity,
   in the colon epithelium thereof, such that there is a cancer phenotype in the colon epithelium of the D. melanogaster;
ii) contacting the D. melanogaster with each of the first compound and the second compound;
iii) determining whether there is a difference between the cancer phenotype of the D. melanogaster of ii) and the cancer phenotype of a corresponding D. melanogaster not contacted with the first compound and the second compound; and
vi) identifying the combination of the first compound and the second compound as likely to be useful for the treatment of epithelial cancer if there is a difference between the cancer phenotype of the at least one D. melanogaster contacted with the first compound and the second compound and the cancer phenotype of the corresponding D. melanogaster not contacted with the first compound and the second compound.

In some embodiments, in step ii) the D. melanogaster is contacted with the first compound and the second compound concurrently.

In some embodiments, in step ii) the D. melanogaster is contacted with the first compound before the second compound.

In some embodiments, the first compound is a proteasome antagonist.

In some embodiments, the second compound is a PI3K signal transduction pathway antagonist.

The present invention provides a process of producing a cancer drug comprising steps i) to iv), followed by
v) producing the compound identified in step iv), thereby producing the cancer drug.

In some embodiments, contacting the D. melanogaster with a compound comprises feeding the compound to the D. melanogaster.

In some embodiments, the D. melanogaster is an adult D. melanogaster.

In some embodiments, the D. melanogaster is genetically modified to have
a) increased Ras activity, and
b) a reduced level of p53, PTEN, or APC expression or activity,
   in the colon epithelium thereof.

In some embodiments, the D. melanogaster is genetically modified to have
a) increased Ras activity, and
b) a reduced level of PTEN expression or activity
   in the colon epithelium thereof.

In some embodiments, the D. melanogaster is genetically modified to have
a) increased Ras activity, and
b) increased PI3K activity,
   in the colon epithelium thereof.

In some embodiments, the difference between the cancer phenotype of the D. melanogaster contacted with the compound and the cancer phenotype of the corresponding D. melanogaster not contacted with the compound is in the colon epithelium.

In some embodiments, the difference in the colon epithelium comprises one or more of reduced
a) proliferation of epithelial cells in the colon epithelium;
b) evasion of apoptosis by epithelial cells in the colon epithelium;
c) disruption of the architecture of the colon epithelium;
d) loss of epithelial characteristics of epithelial cells in the colon epithelium;
e) extension of membrane processes toward the basement membrane of epithelial cells in the colon epithelium;
f) delamination of epithelial cells in the colon epithelium from the colon epithelium;
g) migration of epithelial cells of the colon epithelium away from the colon epithelium;
h) migration of epithelial cells of the colon epithelium into the abdominal cavity;
i) migration of epithelial cells of the colon epithelium into the head or at least one leg;
j) cell membrane-localized pSrc in epithelial cells in the colon epithelium;
k) MMPL expression in epithelial cells in the colon epithelium; or
l) degradation of the basement membrane in the colon epithelium
   of the D. melanogaster contacted with the compound compared to the epithelium of the corresponding D. melanogaster not contacted with the compound.

In some embodiments, the difference in the colon epithelium comprises one or more of increased
a) epithelial cell apoptosis in the colon epithelium;
b) senescence in epithelial cells in the colon epithelium; or
c) laminin expression in epithelial cells in the colon epithelium
   of the D. melanogaster contacted with the compound compared to the epithelium of the corresponding D. melanogaster not contacted with the compound.

In some embodiments, the difference between the cancer phenotype of the D. melanogaster contacted with the first compound and the second compound and the cancer phenotype of the corresponding D. melanogaster not contacted with the first compound and the second compound is in the colon epithelium.

In some embodiments, the difference in the colon epithelium comprises one or more of reduced
a) proliferation of epithelial cells in the colon epithelium;
b) evasion of apoptosis by epithelial cells in the colon epithelium;
c) disruption of the architecture of the colon epithelium;
d) loss of epithelial characteristics of epithelial cells in the colon epithelium;
e) extension of membrane processes toward the basement membrane of epithelial cells in the colon epithelium;
f) delamination of epithelial cells in the colon epithelium from the colon epithelium;
g) migration of epithelial cells of the colon epithelium away from the colon epithelium;
h) migration of epithelial cells of the colon epithelium into the abdominal cavity;
i) migration of epithelial cells of the colon epithelium into the head or at least one leg;
j) cell membrane-localized pSrc in epithelial cells in the colon epithelium;
k) MMP1 expression in epithelial cells in the colon epithelium; or
l) degradation of the basement membrane in the colon epithelium
   of the D. melanogaster contacted with the first compound and the second compound compared to the corresponding D. melanogaster not contacted with the first compound and the second compound.

In some embodiments, the difference between the colon epithelium of the D. melanogaster of ii) and the colon epithelium of the corresponding D. melanogaster not contacted with the compounds comprises one or more of increased
a) epithelial cell apoptossis in the colon epithelium;
b) senescence in epithelial cells in the colon epithelium; or
c) laminin expression in epithelial cells in the colon epithelium
   of the D. melanogaster contacted with the first compound and the second compound compared to the corresponding D. melanogaster not contacted with the first compound and the second compound.

In some embodiments, the epithelial cell cancer is colon cancer.

The present invention provides a process for identifying whether a compound is an epithelial cancer drug candidate comprising
i) obtaining an epithelial cell which has
   a) increased Ras activity,
   b) increased PI3K activity, and/or
   c) a reduced level of p53, PTEN, or APC expression or activity,
   such that the epithelial cell has a cancer phenotype;
ii) contacting the epithelial cell with the compound;
iii) determining whether there is a difference between the cancer phenotype of the epithelial cell of ii) and the cancer phenotype of a corresponding epithelial cell not contacted with the compound; and
vi) identifying the compound as an epithelial cancer drug candidate if there is a difference between the cancer phenotype of the epithelial cell contacted with the compound and the cancer phenotype of the corresponding epithelial cell not contacted with the compound.

The present invention provides a process for identifying whether a combination of a first compound and a second compound is likely to be useful for the treatment of an epithelial cancer comprising
i) obtaining an epithelial cell which has
   a) increased Ras activity,
   b) increased PI3K activity, and/or
   c) a reduced level of p53, PTEN, or APC expression or activity,
   such that the epithelial cell has a cancer phenotype;
ii) contacting the epithelial cell with each of the first compound and the second compound;
iii) determining whether there is a difference between the cancer phenotype of the epithelial cell of ii) and the cancer phenotype of a corresponding epithelial cell not contacted with the first compound and the second compound; and
vi) identifying the combination of the first compound and the second compound as likely to be useful for the treatment of epithelial cancer if there is a difference between the cancer phenotype of the at least one epithelial cell contacted with the first compound and the second compound and the cancer phenotype of the corresponding epithelial cell not contacted with the first compound and the second compound.

In some embodiments, in step ii) the epithelial cell is contacted with the first compound and the second compound concurrently.

In some embodiments, in step ii) the epithelial cell is contacted with the first compound before the second compound.

In some embodiments, the first compound is a proteasome antagonist.

In some embodiments, the second compound is a PI3K signal transduction pathway antagonist.

The present invention provides a process of producing a cancer drug comprising steps i) to iv), followed by
v) producing the compound identified in step iv), thereby producing the cancer drug.

In some embodiments, the epithelial cell has
a) increased Ras activity, and
b) a reduced level of p53, PTEN, or APC expression or activity.

In some embodiments, the epithelial cell has
a) increased Ras activity, and
b) a reduced level of PTEN expression or activity.

In some embodiments, the epithelial cell has
a) increased Ras activity, and
b) increased PI3K activity.

In some embodiments, the difference between the cancer phenotype of the epithelial cell contacted with the compound and the cancer phenotype of the corresponding epithelial cell not contacted with the compound comprises reduced proliferation in the epithelial cell contacted with the compound compared to the epithelium of the corresponding epithelial cell not contacted with the compound.

In some embodiments, the difference between the cancer phenotype of the epithelial cell contacted with the compound and the cancer phenotype of the corresponding epithelial cell not contacted with the compound comprises at least one of increased apoptosis or senescence in the epithelial cell contacted with the compound compared to the epithelium of the corresponding epithelial cell not contacted with the compound.

In some embodiments, the difference between the cancer phenotype of the epithelial cell contacted with the compound and the cancer phenotype of the corresponding epithelial cell not contacted with the compound comprises reduced proliferation in the epithelial cell contacted with the first compound and the second compound compared to the corresponding epithelial cell not contacted with the first compound and the second compound.

In some embodiments, the difference between the cancer phenotype of the epithelial cell contacted with the compound and the cancer phenotype of the corresponding epithelial cell not contacted with the compound comprises at least one of increased apoptosis or senescence in the epithelial cell contacted with the first compound and the second compound compared to the corresponding epithelial cell not contacted with the first compound and the second compound.

In some embodiments, the epithelial cell is an animal cell.

In some embodiments, the epithelial cell is a mammalian cell.

In some embodiments, the epithelial cell is a human cell.

In some embodiments, the epithelial cell has been genetically modified to have
a) increased Ras activity,
b) increased PI3K activity, and/or
c) a reduced level of p53, PTEN, or APC expression or activity.

In some embodiments, the epithelial cell is a cancer cell.

In some embodiments, the cancer cell is a colon cancer cell.

The present invention provides a method for identifying a cancer patient who will likely benefit from treatment with a PI3K signal transduction pathway antagonist comprising
i) obtaining a biological sample comprising cancer tissue from the cancer patient;
ii) detecting whether the cancer tissue in the biological sample
   a) has increased Ras activity and
      α) increased PI3K activity, or
      β) reduced PTEN expression or activity, or
   b) has an increased amount of pAkt and a reduced level of TORC1 activity,
   compared to normal tissue of the same type; and
iii) identifying the cancer patient as a cancer patient who will likely benefit from treatment with a PI3K signal transduction pathway antagonist if in step (ii) neither
   a) increased Ras activity and
      α) increased PI3K activity, or
      β) reduced PTEN expression or activity, nor
   b) an increased amount of pAkt and a reduced level of TORC1 activity,
   is detected in cancer tissue in the biological sample, and identifying the cancer patient as a cancer patient who will not likely benefit from treatment with a PI3K signal transduction pathway antagonist if in step (ii) either
   a) increased Ras activity and
      α) increased PI3K activity, or
      β) reduced PTEN expression or activity, or
   b) an increased amount of pAkt and a reduced level of TORC1 activity,
   is detected in cancer tissue in the biological sample.

In some embodiments, the cancer patient is selected from the group of cancer patients having colon cancer.

The present invention provides a method of treating a cancer patient identified to not likely benefit from treatment with a PI3K signal transduction pathway antagonist comprising the method of the invention.

The present invention provides a kit for identifying a cancer patient who will likely benefit from treatment with a PI3K signal transduction pathway antagonist comprising
i) at least one probe or primer for determining
   a) whether PTEN expression is reduced; or
   b) whether there is a mutation in PTEN reduces the activity thereof,
   in a biological sample, or from nucleic acid obtained from a biological sample, and/or
ii) at least one probe or primer for determining whether there is a mutation in Ras that increases the activity thereof,
   in a biological sample, or from nucleic acid obtained from the biological sample, and/or
iii) at least one probe or primer for determining whether there is a mutation in PI3K that increases the activity thereof,
   in a biological sample, or from nucleic acid obtained from the biological sample, and/or
iv) at least one antibody for determining the amount of p-4EBP in a biological sample, or in protein obtained from the biological sample, and/or
v) at least one antibody for determining the amount of pAKT in a biological sample, or in protein obtained from the biological sample.

In some embodiments, the kit further comprises instructions for use.

In some embodiments, the kit comprises
i) at least one probe or primer for determining whether there is a mutation in Ras that increases the activity thereof,
   in a biological sample, or from nucleic acid obtained from the biological sample, and
ii) at least one antibody for determining the amount of p-4EBP in the biological sample, or in protein obtained from the biological sample.

In some embodiments, the amount of the proteasome antagonist and the amount of the PI3K signal transduction pathway antagonist when administered in combination is more effective to treat the subject than would be expected based on the additive effects of each agent administered alone.

In some embodiments, the Wnt signal transduction pathway, Receptor Tyrosine Kinase (RTK)/Ras signal transduction pathway, p53 signal transduction pathway, TGF-β signal transduction pathway, or PI3K signal transduction pathway is misregulated in cells of the cancer.

In some embodiments, at least two of the Wnt signal transduction pathway, Receptor Tyrosine Kinase (RTK)/Ras signal transduction pathway, p53 signal transduction pathway, TGF-β signal transduction pathway, and PI3K signal transduction pathway are misregulated in cells of the cancer.

In some embodiments, cells of the cancer have a reduced level of p53, PTEN, or APC protein expression.

In some embodiments, cells of the cancer express p53, PTEN, or APC protein with reduced activity.

Each embodiment disclosed herein is contemplated as being applicable to each of the other disclosed embodiments. Thus, all combinations of the various elements described herein are within the scope of the invention.

It is understood that where a parameter range is provided, all integers within that range are disclosed. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Terms

“About” in the context of a numerical value or range means±10% of the numerical value or range recited or claimed, unless the context requires a more limited range.

As used herein, “PI3K signal transduction pathway” includes any polypeptide or complex of polypeptides that physically interacts with or is a component of PI3K in a cell, and any polypeptide or complex of polypeptides that is downstream of PI3K singaling such that its level of phosphorylation, activation, binding activity, and/or catalytic rate may be directly or indirectly modulated by PI3K catalytic activity. Non-limiting examples of members of the PI3K signal transduction pathway are AKT, mTor, TORC1, TORC2, AKT, and JNK.

As used herein, a “PI3K signal transduction pathway antagonist” includes any compound that binds to and reduces the phosphorylation, activation, binding activity, and/or catalytic rate of a member of the PI3K signal transduction pathway.

As used herein, a “proteasome antagonist” includes any compound that reduces proteasome function. In some embodiments, a proteasome antagonist binds to one or more polypeptides of a proteasome.

The amino acid sequence of p110α is accessible in public databases by the accession numbers NP_{—}006209.2 and P42336, and CCDS number CCDS43171.1, and is set forth herein as SEQ ID NO: 1. Nucleotide sequences for p110α cDNA and the sequence of the PIK3CA gene are accessible in public databases, e.g. from the Gene ID for PIK3CA (or p110α), which is Gene ID 5290.

Amino acid sequences of K-Ras are accessible in public databases by the accession numbers P01116 (Isoform 2A (identifier: P01116-1); Isoform 2B (identifier: P01116-2)) and NP_{—}004976.2, and CCDS number CCDS8702.1, and are set forth herein as SEQ ID NOs: 2 and 3. Nucleotide sequences for for K-Ras cDNA and the sequence of the K-Ras gene are accessible in public databases, e.g. from the Gene ID for K-Ras, which is Gene ID 3845.

The amino acid sequence of N-Ras is accessible in public databases by the accession number P01111 and NP_{—}002515.1, and is set forth herein as SEQ ID NO: 4. Nucleotide sequences for for N-Ras cDNA and the sequence of the N-Ras gene are accessible in public databases, e.g. from the Gene ID for N-Ras, which is Gene ID 4893.

Amino acid sequences of H-Ras are accessible in public databases by the accession numbers P01112 and NP_{—}001123914.1 (Isoform 1), and P01112-2 and (Isoform 2), and are set forth herein as SEQ ID NOS: 5 and 6. Nucleotide sequences for for H-Ras cDNA and the sequence of the H-Ras gene are accessible in public databases, e.g. from the Gene ID for H-Ras, which is Gene ID 3265.

The amino acid sequence of PTEN is accessible in public databases by the accession numbers P60484 and NP_{—}000305.3 and is set forth herein as SEQ ID NO: 7. Nucleotide sequences for for PTEN cDNA and the sequence of the PTEN gene are accessible in public databases, e.g. from the Gene ID for PTEN, which is Gene ID 5728.

The amino acid sequence of mTor is accessible in public databases by the accession numbers P42345 and NP_{—}004949.1, and is set forth herein as SEQ ID NO: 8. Nucleotide sequences for for mTor cDNA and the sequence of the mTor gene are accessible in public databases, e.g. from the Gene ID for mTor, which is Gene ID 2475.

The amino acid sequence of AKT1 is accessible in public databases by the accession numbers P31749 and NP_{—}001014431.1, and is set forth herein as SEQ ID NO: 9. Nucleotide sequences for AKT1 cDNA and the sequence of the AKT1 gene are accessible in public databases, e.g. from the Gene ID for AKT1, which is Gene ID 207.

The amino acid sequence of AKT2 is accessible in public databases by the accession numbers P31751 and NP_{—}001617.1, and is set forth herein as SEQ ID NO: 10. Nucleotide sequences for for AKT2 cDNA and the sequence of the AKT2 gene are accessible in public databases, e.g. from the Gene ID for AKT2, which is Gene ID 208.

Amino acid sequences of JNK are accessible in public databases by the accession numbers P45983 (Isoform 2), P45983-2 (Isoform 1), P45983-3 (Isoform 3), P45983-4 (Isoform 4), and are set forth herein as SEQ ID NOs: 11-14, respectively. Nucleotide sequences for JNK cDNA and the sequence of the JNK gene are accessible in public databases, e.g. from the Gene ID for JNK, which is Gene ID 5599.

It will be understood that mutations, including activating and loss of function mutations in any of Ras, AKT, JNK, mTor, PTEN, p110α, or any other polypeptide disclosed herein may be identified using methods that are well known in the art. For example, PTEN may be inactivated by deletions and loss of function mutations. Kits for identifying PTEN mutants are commercially available. One kit that detects 20 most commonly observed mutations in PTEN is available from SA Biosciences (a Qiagen Company, Valencia, Calif., USA) at www.sabiosciences.com/qbiomarker_product/HTML/SMH-809A.html the entire contents of this reference are incorporated herein by reference.

Aspects of the present invention relate to a PI3K signal transduction pathway antagonist having the structure:

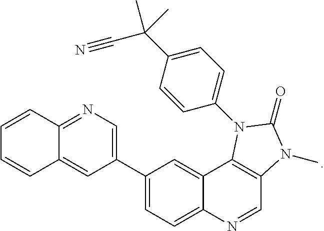 [SMILES: Cn1c(=O)n(-c2ccc(C(C)(C)C#N)cc2)c2c3cc(-c4cnc5ccccc5c4)ccc3ncc21]

This compound is also known as BEZ235 and NVP-BEZ235 and BEZ-235. BEZ235 is available from LC Labs (Woburn, Mass., USA). The PubChem CID number for BEZ235 is 11977753 and the CAS number for BEZ253 is 915019-65-7. The molecular formula for BEZ235 is C₃₀H₂₃N₅O. BEZ235 is discussed in Liu et al., (2009) “NVP-BEZ235, a novel dual phosphatidylinositol 3-kinase/mammalian target of rapamycin inhibitor, elicits multifaceted antitumor activities in human gliomas” Mol Cancer Ther August 2009 8; 2204-2210, the entire contents of which are incorporated herein by reference.

Aspects of the present invention relate to a PI3K signal transduction pathway antagonist having the structure:

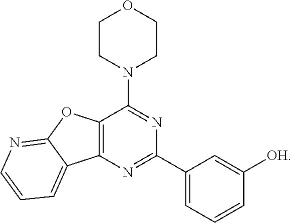 [SMILES: Oc1cccc(-c2nc(N3CCOCC3)c3oc4ncccc4c3n2)c1]

This compound is also known as PI-103. PI-103 is available from Tocris (Bristol, United Kingdom). The PubChem CID number for PI-103 is 9884685 and the CAS number for PI-103 is 371935-74-9. The molecular formula for PI-103 is C₁₉H₁₆N₄O₃. PI-103 is discussed in Fan et al. (2006) “A dual PI3 kinase/mTOR inhibitor reveals emergent efficacy in glioma.” Cancer Cell, 9(5):341-9, the entire contents of which are incorporated herein by reference.

Aspects of the present invention relate to a PI3K signal transduction pathway antagonist having the structure:

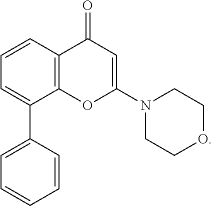 [SMILES: O=c1cc(N2CCOCC2)oc2c(-c3ccccc3)cccc12]

This compound is also known as LY294002. LY294002 is available from LC Labs (Woburn, Mass., USA). The PubChem CID number for LY294002 is 3973 and the CAS number for LY294002 is 154447-36-6. The molecular formula for LY294002 is C₁₉H₁₇NO₃. LY294002 is discussed in Vlahos et al. (1994) “A specific inhibitor of phosphatidylinositol 3-kinase, 2-(4-morpholinyl)-8-phenyl-4H-1-benzopyran-4-one (LY294002)” J Biol Chem, 269(7):5241-8., and Imai et al. (2012) “The PI3K/Akt inhibitor LY294002 reverses BCRP-mediated drug resistance without affecting BCRP translocation.” Oncol Rep, 27(6):1703-9, the entire contents of each of which are incorporated herein by reference.

Aspects of the present invention relate to a PI3K signal transduction pathway antagonist having the structure:

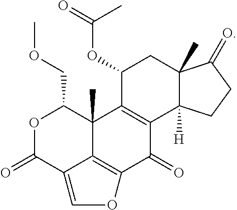 [SMILES: COC[C@H]1OC(=O)c2coc3c2[C@@]1(C)C1=C(C3=O)[C@@H]2CCC(=O)[C@@]2(C)C[C@H]1OC(C)=O]

This compound is also known as wortmannin. Wortmannin is available from LC Labs (Woburn, Mass., USA). The PubChem CID number for wortmannin is 312145 and the CAS number for wortmannin is 19545-26-7. The molecular formula for wortmannin is C₂₃H₂₄O₈. Wortmannin is discussed in Arcaro and Wymann (1993) “Wortmannin is a potent phosphatidylinositol 3-kinase inhibitor: the role of phosphatidylinositol 3,4,5-trisphosphate in neutrophil responses.” Biochem J, 296 (Pt 2):297-301, the entire contents of which are incorporated herein by reference.

Aspects of the present invention relate to a PI3K signal transduction pathway antagonist binds to JNK and having the structure:

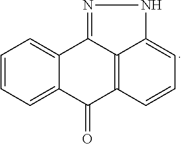 [SMILES: O=C1c2ccccc2-c2n[nH]c3cccc1c23]

This compound is also known as SP600125. SP600125 is available from LC Labs (Woburn, Mass., USA). The PubChem CID number for SP600125 is 8515 and the CAS number for SP600125 is 129-56-6. The molecular formula for SP600125 is C₁₄H₈N₂O. SP600125 is discussed in Bennett et al. (2001) “SP600125, an anthrapyrazolone inhibitor of Jun N-terminal kinase.” Proc Natl Acad Sci USA, 98(24):13681-6, the entire contents of which are incorporated herein by reference.

Aspects of the present invention relate to a proteasome antagonist having the structure:

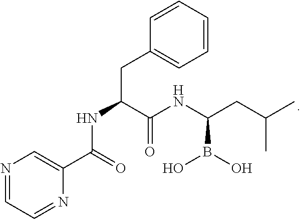 [SMILES: CC(C)C[C@H](NC(=O)[C@H](Cc1ccccc1)NC(=O)c1cnccn1)B(O)O]

This compound is also known as bortezomib, PS-341, and LDP-341. Bortezomib is available from LC Labs (Woburn, Mass., USA). The PubChem CID number for bortezomib is 387447 and the CAS number for bortezomib is 179324-69-7. The molecular formula for bortezomib is C₁₉H₂₅BN₄O₄. Bortezomib is discussed in Nawrocki et al. (2005) “Bortezomib inhibits PKR-like endoplasmic reticulum (ER) kinase and induces apoptosis via ER stress in human pancreatic cancer cells.” Cancer Res, 65(24):11510-9, the entire contents of which are incorporated herein by reference.

Aspects of the present invention relate to a proteasome antagonist having the structure:

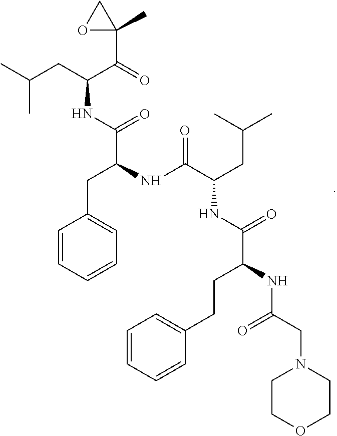 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

This compound is also known as carfilzomib. Carfilzomib is available from Active Biochem (Maplewood, N.J., USA; Cat#A-1098). The PubChem CID number for carfilzomib is 11556711 and the CAS number for carfilzomib is 868540-17-4. The molecular formula for carfilzomib is C₄₀H₅₇N₅O₇. Carfilzomib is discussed in Kuhn et al. (2007) “Potent activity of carfilzomib, a novel, irreversible inhibitor of the ubiquitin-proteasome pathway, against preclinical models of multiple myeloma.” Blood, 110(9):3281-90, the entire contents of which are incorporated herein by reference.

### Organic Compound Structure I

In some embodiments, the PI3K signal transduction pathway antagonist is an organic compound that binds PI3K having the structure Ia, Ib, or Ic:

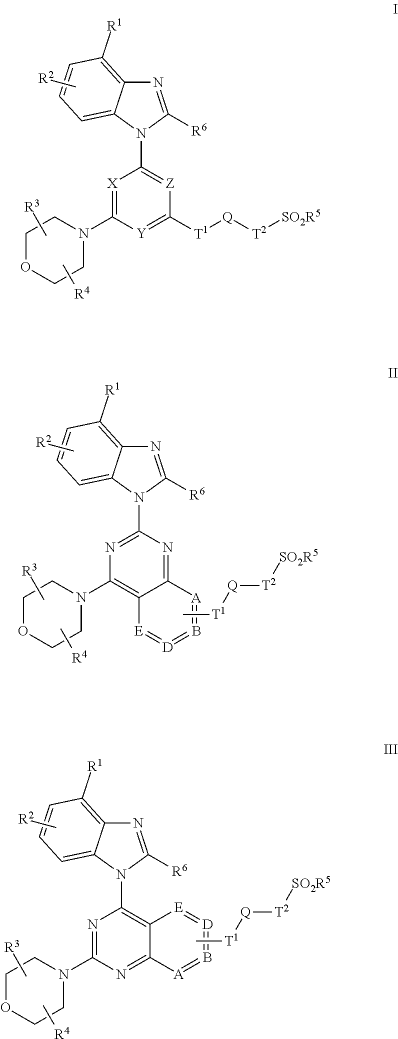

or an enantiomer, a mixture of enantiomers, or a mixture of two or more diastereomers thereof; or a pharmaceutically acceptable salt, solvate, hydrate, or prodrug thereof; wherein:
each R¹ and R² is independently (a) hydrogen, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1b}, —C(O)NR^{1b}R^{1c}, —C(NR^{a})NR^{1b}R^{1c}, —OR^{1a}, —OC(O)R^{1a}, —OC(O)OR^{1a}, —OC(O)NR^{1b}R^{1c}, OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiments, one two three or four, substituents Q¹; or (iii) R^{1b} and R^{1c} together with the N atom to which they are attached form heterocyclyl, optionally substituted with one or more, in one embodiment, one two three or four, substituents Q¹;
   each R³ and R⁴ is independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
   each R⁵ is independently C₁₋₆ alkyl, C₂₋₆ alkenyl, C₂₋₆ alkynyl, C₃₋₇ cycloalkyl, C₃₋₇ cycloalkyl, C₆₋₁₄ aryl, C₇₋₁₅ aralkyl, heteroaryl, or heterocyclyl;
   each R⁶ in independently hydrogen or C₁₋₆ alkyl;
   each A, B, D, and E is independently (i) a bond; (ii) a nitrogen, oxygen, or sulfur atom; or (iii) CR⁷, where R⁷ is hydrogen, halo, C₁₋₆ alkyl, C₂₋₆ alkenyl, or C₂₋₆ alkynyl; wherein the bonds between A, B, D, and E may be saturated or unsaturated; with the proviso that no more than one of A, B, D, and E are a bond;
   each Q is C₁₋₆ alkylene, C₂₋₆ alkenylene, C₂₋₆ alkynylene, C₃₋₇ cycloalkylene, C₆₋₁₄ arylene, heteroarylene, or heterocyclylene;
   each T¹ is independently a bond, —O—, or —NR⁸—;
   each T² is independently a bond or —NR⁸—, with the proviso that the atom that is attached to —SO₂R⁵ is nitrogen;
   each R⁸ is independently hydrogen, C₁₋₆ alkyl, C₂₋₆ alkenyl, or C₂₋₆ alkynyl; and
   X, Y, and Z are each independently a nitrogen atom or CR⁹, with the proviso that at least two of X, Y, and Z are nitrogen atoms; where R⁹ is hydrogen or C₁₋₆ alkyl;
   wherein each alkyl, alkylene, alkenyl, alkenylene, alkynyl, alkynylene, cycloalkyl, cycloalkylene, aryl, arylene, heteroaryl, heteroarylene, heterocyclyl, and heterocyclylene is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)N^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;
   wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)N^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}; R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Organic compounds of this structure, as well as processes of synthesizing organic compounds of this structure are described in U.S. Patent Application Publication No. US 2010/0249099, the entire contents of each of which are incorporated herein by reference.

### Organic Compound Structure II

In some embodiments, the PI3K signal transduction pathway antagonist is an organic compound that binds PI3K having the structure IIa, IIb, IIa, or IId:

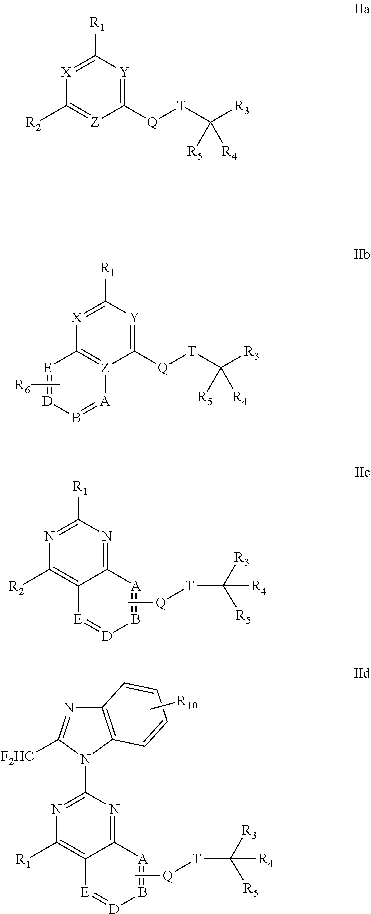

or an enantiomer, a mixture of enantiomers, or a mixture of two or more diastereomers thereof; or a pharmaceutically acceptable salt, solvate, hydrate, or prodrug thereof; wherein:
each R₁ is independently C₆-14 aryl, heteroaryl, or heterocyclyl;
each R₂ is independently C₆₋₁₄ aryl, heteroaryl, or heterocyclyl;
each R₃ and R₄ is independently hydrogen, lower alkyl, C₂₋₆ alkenyl, C₂₋₆ alkynyl, or R₅;
each R₅ is independently halogen or —OSO₂R₇;
R₆ is C₃₋₇ cycloalkyl, C₆₋₁₄ aryl, heteroaryl, or heterocyclyl;
R₇ is lower alkyl, C₂₋₆ alkenyl, C₂₋₆ alkynyl, C₃₋₇ cycloalkyl, C₆₋₁₄ aryl, heteroaryl, or heterocyclyl;
R₁₀ is (a) hydrogen, amino, or hydroxyl; or (b) lower alkyl, lower alkylamino, di(lower alkyl)amino, lower alkoxy, or carboxamido;
each Q is independently absent or a linker group;
each T is independently —CO—, —CS—, or —SO2-;
X, Y, and Z are each independently a nitrogen atom or CR₈, with the proviso that at least two of X, Y, and Z are nitrogen atoms; wherein R₈ is hydrogen or lower alkyl; and
each A, B, D, and E is independently (i) a direct bond; (ii) a nitrogen, oxygen, or sulfur atom; or (iii) CR₉, where R₉ is hydrogen, halogen, or lower alkyl; wherein the bonds between A, B, D, and E may be saturated or unsaturated; with the proviso that no more than one of A, B, D, and E are a direct bond;
wherein each alkyl, alkenyl, alkynyl, alkoxy, alkylamino, dialkylamino, carboxamido, cycloalkyl, aryl, heteroaryl, and heterocyclyl is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)NR^{b}R^{c}, C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;
wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}; R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Organic compounds of this structure, as well as processes of synthesizing organic compounds of this structure are described in U.S. Patent Application Publication No. US 2011/0053907, the entire contents of each of which are incorporated herein by reference.

### Organic Compound Structure III

In some embodiments, the PI3K signal transduction pathway antagonist is an organic compound that binds PI3K having the structure:

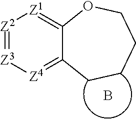

stereoisomers, geometric isomers, tautomers, and pharmaceutically acceptable salts thereof, wherein:
B is a pyrazolyl, imidazolyl, or triazolyl ring fused to the benzoxepin ring and selected from the structures:

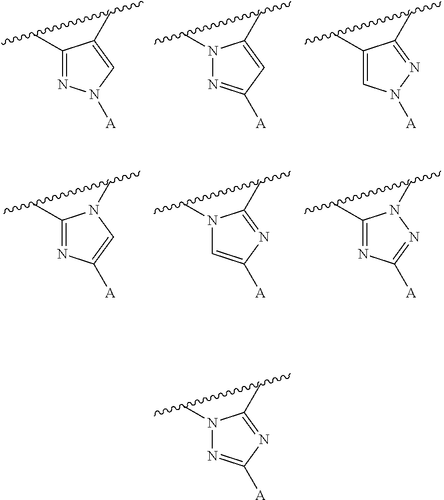

Z¹ is CR¹ or N;
Z² is CR² or N;
Z³ is CR³ or N;
Z⁴ is CR⁴ or N;
R¹, R², R³, and R⁴ are independently selected from H, F, Cl, Br, I, —CN, —COR¹⁰, —CO₂R¹⁰, —C(═O)N(R¹⁰)OR¹¹, —C(═NR¹⁰)NR¹⁰R¹¹, —C(═O)NR¹⁰R¹¹, —NO₂, —NR¹⁰R¹¹, —NR¹²C(O)R¹⁰, —NR¹²C(═O)OR¹¹, —NR¹²C(═O)NR¹⁰R¹¹, —NR¹²C(═O)(C₁-C₁₂ alkylene)NR¹⁰R¹¹, NR¹²(C₁-C₁₂ alkylene)NR¹⁰R¹¹, —NR¹²(C₁-C₁₂alkylene)OR¹⁰, —NR¹²(C₁-C₁₂ alkylene)C(═O)NR¹⁰R¹¹, —OR¹⁰, —SR¹⁰, —S(O)₂R¹⁰,
—C(═O)NR¹⁰(C₁-C₁₂ alkylene)NR¹⁰R¹¹,
—C(═O)NR¹⁰(C₁-C₁₂ alkylene)NR¹⁰C(═O)OR¹¹,
—C(═O)NR¹⁰(C₁-C₁₂ alkylene)NR¹⁰C(═O)R¹¹,
—C(═O)NR¹⁰(C₁-C₁₂ alkylene)R¹⁰,
C₁-C₁₂ alkyl,
C₂-C₈ alkenyl,
C₂-C₈ alkynyl,
C₃-C₁₂ carbocyclyl,
C₂-C₂, heterocyclyl,
C₆-C₂₀ aryl,
C₁-C₂₀ heteroaryl,
—(C₃-C₁₂ carbocyclyl)-(C₁-C₁₂ alkyl),
—(C₂-C₂₀ heterocyclyl)-(C₁-C₁₂ alkyl),
—(C₆-C₂₀ aryl)-(C₁-C₁₂ alkyl),
—(C₁-C₂₀ heteroaryl)-(C₁-C₁₂ alkyl),
—(C₁-C₁₂ alkylene)-(C₃-C₁₂ carbocyclyl),
—(C₁-C₂ alkylene)-(C₂-C₂₀ heterocyclyl),
—(C₁-C₁₂ alkylene)-(C₂-C₂₀ heterocyclyl)-(C₂-C₂₀ heterocyclyl),
—(C₁-C₁₂ alkylene)-(C₂-C₂₀ heterocyclyl)-(C₃-C₁₂ carbocyclyl),
—(C₁-C₁₂ alkylene)-(C₂-C₂₀ heterocyclyl)-C(═O)—(C₂-C₂₀ heterocyclyl),
—(C₁-C₁₂ alkylene)-(C₁-C₂₀ heteroaryl),
—(C₁-C₁₂ alkylene)-(C₂-C₂₀ heterocyclyl)-(C₁-C₁₂ alkyl),
—(C₁-C₁₂ alkylene)-(C₆-C₂₀ aryl)-(C₁-C₁₂ alkyl),
—(C₁-C₁₂ alkylene)-(C₁-C₂₀ heteroaryl)-(C₁-C₁₂ alkyl),
—(C₁-C₁₂ alkylene)-C(═O)—(C₂-C₂₀ heterocyclyl),
—(C₁-C₁₂ alkylene)C(═O) OR¹⁰,
—(C₁-C₁₂ alkylene)C(═O) NR¹⁰R¹¹,
—(C₁-C₁₂ alkylene)-NR¹⁰R¹¹,
—(C₁-C₁₂ alkylene)NR¹²C(═O)R¹⁰,
—(C₁-C₁₂ alkylene)OR¹⁰,
—(C₁-C₁₂ alkylene)-NR¹⁰—(C₁-C₁₂ alkylene)-(C₁-C₂₀ heteroaryl),
—(C₁-C₁₂ alkylene)-NR¹⁰—(C₁-C₁₂ alkylene)-(C₁-C₂₀ heterocyclyl),
—(C₁-C₁₂ alkylene)-NR₁₀—(C₁-C₁₂ alkylene)-NHC(═O)—(C₁-C₂₀ heteroaryl),
—(C₁-C₁₂ alkylene)-(C₂-C₂₀ heterocyclyl)-NR¹⁰R¹¹, and
—(C₁-C₁₂ alkylene)-(C₂-C₂₀ heterocyclyl)-(C₁-C₁₂ alkyl)-NR¹⁰R¹¹,
where alkyl, alkenyl, alkynyl, alkylene, carbocyclyl, heterocyclyl, aryl, and heteroaryl are optionally substituted with one or more groups independently selected from F, Cl, Br, I, R¹⁰, —SR¹⁰, —S(O)₂R¹⁰, —S(O)₂NR¹⁰R¹¹, NR¹⁰R¹¹, —NR¹²C(O)R¹⁰, CO₂R¹⁰, —C(O)R¹⁰, —CONR¹⁰R¹¹, oxo, and —OR¹⁰;
A is selected from —C(═O)NR⁵R⁶, —NR⁵R⁶, C₆-C₂₀ aryl, C₂-C₂₀heterocyclyl and C₁-C₂₀ heteroaryl wherein aryl, heterocyclyl and heteroaryl are optionally substituted with one or more groups independently selected from F, Cl, Br, I, —CN, —COR¹⁰, —CO₂R¹⁰, —C(═O)N(R¹⁰)OR¹¹, —C(═NR¹⁰)NR¹⁰R¹¹, —C(═O)NR¹⁰R¹¹, —NO₂, —NR¹⁰R¹¹, —NR¹²C(═O)R¹⁰, —NR¹²C(═O)OR¹¹, —NR¹²C(═O)NR¹⁰R¹¹, —NR¹²C(═O)(C₁-C₁₂ alkylene)NR¹⁰R¹¹, —NR¹²(C₁-C₁₂ alkylene)NR¹⁰R¹¹, —NR¹²(C₁-C₁₂ alkylene)OR¹⁰, —NR¹²(C₁-C₁₂ alkylene)C(═O)NR¹⁰R¹¹, —OR¹⁰, —S(O)₂R¹⁰,
—C(═O)NR¹⁰(C₁-C₁₂ alkylene)NR¹⁰R¹¹,
—C(═O)NR¹⁰ (C₁-C₁₂ alkylene)NR¹⁰C(═O)OR¹¹,
—C(═O)NR¹⁰(C₁-C₁₂ alkylene)NR¹⁰C(═O)R¹¹,
—C(═O)NR¹⁰(C₁-C₁₂ alkylene)R¹⁰,
C₁-C₁₂ alkyl,
C₂-C₈ alkenyl,
C₂-C₈ alkynyl,
C₃-C₁₂ carbocyclyl,
C₂-C₂₀ heterocyclyl,
C₆-C₂₀ aryl,
C₁-C₂₀ heteroaryl,
—(C₃-C₁₂ carbocyclyl)-(C₁-C₁₂ alkyl),
—(C₂-C₂₀ heterocyclyl)-(C₁-C₁₂ alkyl),
—(C₆-C₂₀ aryl)-(C₁-C₁₂ alkyl),
—(C₁-C₂₀ heteroaryl)-(C₁-C₁₂ alkyl),
—(C₁-C₁₂ alkylene)-(C₃-C₁₂ carbocyclyl),
—(C₁-C₁₂ alkylene)-(C₂-C₂₀ heterocyclyl),
—(C₁-C₁₂ alkylene)-(C₂-C₂₀ heterocyclyl)-(C₂-C₂₀ heterocyclyl),
—(C₁-C₁₂ alkylene)-(C₂-C₂₀ heterocyclyl)-(C₃-C₁₂ carbocyclyl),
—(C₁-C₁₂ alkylene)-(C₂-C₂₀ heterocyclyl)-C(═O)—(C₂-C₂₀ heterocyclyl),
—(C₁-C₁₂ alkylene)-(C₁-C₂₀ heteroaryl),
—(C₁-C₁₂ alkylene)-(C₂-C₂₀ heterocyclyl)-(C₁-C₁₂ alkyl),
—(C₁-C₁₂ alkylene)-(C₆-C₂₀ aryl)-(C₁-C₁₂ alkyl),
—(C₁-C₁₂ alkylene)-(C₁-C₂₀ heteroaryl)-(C₁-C₁₂ alkyl),
—(C₁-C₁₂ alkylene)-C(═O)—(C₂-C₂₀ heterocyclyl),
—(C₁-C₁₂ alkylene)C(═O) OR¹⁰,
—(C₁-C₁₂ alkylene)-NR¹⁰R¹¹,
(C₁-C₁₂ alkylene) NR¹²C(═O)R¹⁰,
—(C₁-C₁₂ alkylene)OR¹⁰,
(C₁-C₁₂ alkylene)-NR¹⁰—(C₁-C₁₂ alkylene)-(C₁-C₂₀ heteroaryl),
—(C₁-C₁₂ alkylene)-NR¹⁰—(C₁-C₁₂ alkylene)-(C₁-C₂₀ heterocyclyl),
—(C₁-C₁₂ alkylene)-NR¹⁰—(C₁-C₁₂ alkylene)-NHC(═O)—(C₁-C₂₀heteroaryl),
—(C₁-C₁₂ alkylene)-(C₂-C₂₀ heterocyclyl)-NR¹⁰R¹¹, and
—(C₁-C₁₂ alkylene)-(C₂-C₂₀ heterocyclyl)-(C₁-C₁₂ alkyl)-NR¹⁰R¹¹,
where alkyl, alkenyl, alkynyl, alkylene, carbocyclyl, heterocyclyl, aryl, and heteroaryl are optionally substituted with one or more groups independently selected from F, Cl, Br, I, R¹⁰, —SR¹⁰, —S(O)₂R¹⁰, NR¹⁰R¹¹, —NR¹²C(O)R¹⁰, —CO₂R¹⁰, —CONR¹⁰R¹¹, and —OR¹⁰;
R⁵ is selected from H, and C₁-C₁₂ alkyl, optionally substituted with one or more groups independently selected from F, Cl, Br, I, —CN, —CO₂H, —CONH₂, —CONHCH₃, —NH₂, —NO₂, —N(CH₃)₂, —NHCOCH₃, —NHS(O)₂CH₃, —OH, —OCH₃, —OCH₂CH₃, —S(O)₂NH₂, and —S(O)₂CH₃;
R⁶ is selected from C₁-C₂ alkyl, C₃-C₁₂ carbocyclyl, C₂-C₂₀ heterocyclyl, C₁-C₂₀ heteroaryl, and C₆-C₂₀ aryl, each optionally substituted with one or more groups independently selected from F, Cl, Br, I, —CH₃, —CH₂OH, —CH₂C₆H₅, —CN, —CF₃, —CO₂H, —C(O)CH₃, —NH₂, —NO₂, —N(CH₃)₂, —NHCOCH₃, —NHS(O)₂CH₃, —OH, oxo, —OCH₃, —OCH₂CH₃, —S(O)₂NH₂, —S(O)₂CH₃, —C(═O)NR¹⁰(C₁-C₁₂ alkylene)NR¹⁰R¹¹, phenyl, pyridinyl, tetrahydro-furan-2-yl, 2,3-dihydro-benzofuran-2-yl, 1-isopropyl-pyrrolidin-3-ylmethyl, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, —C≡CR¹³, —CH═CHRS³, and —C(═O)NR¹⁰R¹¹;
or R⁵ and R⁶ together with the nitrogen atom to which they are attached form C₂-C₂₀ heterocyclyl or C₁-C₂₀ heteroaryl, optionally substituted with one or more groups selected from F, Cl, Br, I, CH₃, C(CH₃)₃, —CH₂OH, —CH₂CH₂OH, —CH₂C₆H₅, pyridin-2-yl, 6-methyl-pyridin-2-yl, pyridin-4-yl, pyridin-3-yl, pyrimidin-2-yl, pyrazin-2-yl, tetrahydro-furan-carbonyl, 2-methoxy-phenyl, benzoyl, cyclopropylmethyl, (tetrahydrofuran-2-yl)methyl, 2,6-dimethyl-morpholin-4-yl, 4-methyl-piperazine-carbonyl, pyrrolidine-1-carbonyl, cyclopropanecarbonyl, 2,4-difluoro-phenyl, pyridin-2-ylmethyl, morpholin-4-yl, —CN, —CF₃, —CO₂H, —CONH₂, —CONHCH₃, —CON(CH₃)₂, —COCF₃, —COCH₃, —COCH(CH₃)₂, —NO₂, NHCH₃, —N(CH₃)₂, —N(CH₂CH₃)₂, —NHCOCH₃, —NCH₃COCH₃, —NHS(O)₂CH₃, —OH, —OCH₃, —OCH₂CH₃, —CH₂OCH₃, —CH₂CH₂OCH₃, —CH₂S(O)₂NCH₃, —C₂S(O)₂C₂CH₃, —S(O)₂NHCH₃, —S(O)₂CH₂CH₃, —S(O)₂NH₂, —S(O)₂N(CH₃)₂ and —S(O)₂CH₃;
R¹⁰, R¹¹ and R¹² are independently selected from H, C₁-C₁₂ alkyl, —(C₁-C₁₂ alkylene)-(C₂-C₂₀ heterocyclyl), —(C₁-C₁₂ alkylene)-(C₆-C₂₀ aryl), —(C₁-C₁₂ alkylene)-(C₃-C₁₂ carbocyclyl), C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl, each of which are optionally substituted with one or more groups independently selected from F, Cl, Br, I, —CH₃, —CH₂CH₃, —CH (CH₃)₂, —CH₂OH, —CH₂OCH₃, —CH₂CH₂OH, —C(CH₃)₂OH, —CH₂C(CH₃)₂OH, —CH₂CH(CH₃)OH, —CH₂CO₂H, —CH₂CO₂CH₃, —CH₂NH₂, —(CH₂)₂N(CH₃)₂, —CH₂C₆H₅, —CN, —CF₃, —CO₂H, —C(O)CH₃, —C(O)CH(OH)CH, —CO₂CH₃, —CONH₂, —CONHCH₃, —CON(CH₃)₂, —C(CH₃)₂CONH₂, —NH₂, —NO₂, —N(CH₃)₂, —N(CH₃)C(CH₃)₂CONH₂, —N(CH₃)CH₂CH₂S(O)₂CH₃, —NHCOCH₃, —NHS(O)₂CH₃, ═O(oxo), —OH, —OCH₃, —OCH₂CH₃, —OCH₂CH₂OH, —OP(O)(OH)₂, —SCH₃, —S(O)₂CH₃, —S(O)₂NH₂, —S(O)₂N(CH₃)₂, —CH₂S(O)₂NCH₃, —CH₂S(O)₂CH₂CH₃, —S(O)₂NHCH₃, —S(O)₂CH₂CH₃, pyrrolidin-1-yl, 2-oxopyrrolidin-1-yl, cyclopropyl, cyclopentyl, oxetanyl, 4-methylpiperazin-1-yl, and 4-morpholinyl;
or R¹⁰ and R¹¹ together with the nitrogen atom to which they are attached form a C₂-C₂₀ heterocyclyl ring or C₁-C₂₀ heteroaryl each of which are optionally substituted with one or more groups independently selected from F, Cl, Br, I, —CH₃, —CH₂OH, —CH₂C₆CH₅, —CN, —CF₃, —CO₂H, —CONH₂, —CONHCH₃, —NO₂, —N(CH₃)₂, —NHCOCH₃, —NHS(O)₂CH₃, —OH, oxo, —OCH₃, —OCH₂CH₃, —S(O)₂NH₂, —S(O)₂CH₃, —CH(CH₃)₂, —CH₂CF₃, —CH₂CH₂OH and —C(CH₃)₂OH; and
R¹³ is selected from H, F, Cl, Br, I, —CH₃, —CH₂CH₃, —CN, —CF₃, —CH₂N(CH₃)₂, —CH₂OH, —CO₂H, —CONH₂, —CON(CH₃)₂, —NO₂, and —S(O)₂CH₃.

Organic compounds of this structure, as well as processes of synthesizing organic compounds of this structure are described in U.S. Patent Application Publication No. US 2012/0244149, the entire contents of each of which are incorporated herein by reference.

### Organic Compound Structure IV

In some embodiments, the PI3K signal transduction pathway antagonist is an organic compound that binds PI3K having the structure:

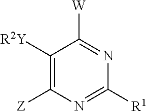

R¹ is selected from:
(i) a group of the following formula:

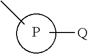

wherein
P is (i) aryl or heteroaryl which is unsubstituted or substituted;
(ii) an indazole group which is unsubstituted or substituted;
(iii) an indole group which is unsubstituted or substituted; or
(iv) a benzoimidazole group which is unsubstituted or substituted;
Q is selected from —H, —OR, —SR, -Halo, —NR₃R₄, —OS(O)ₘR, —OC(O)R, —OC(O)NHR, —S(O)ₘNR₃R₄, —NRC(O)R, —NRS(O)ₘR, —NRC(O)NR₃R₄, and —NRC(S)NR₃R₄, wherein each R, R₃, and R₄ is independently selected from H, C₁-C₆ alkyl, C₃-C₁₀ cycloalkyl and a 5- to 12-membered carbocyclic group, aryl or heteroaryl group, the group being unsubstituted or substituted; m is 1 or 2; or R₃ and R₄, which are the same or different, are each independently selected from H, C₁-C₆ alkyl which is unsubstituted or substituted, C₃-C₁₀ cycloalkyl which is unsubstituted or substituted, —C(O)R, —C(O)N(R)₂ and —S(O)ₘR wherein R and m are as defined above, or R₃ and R₄ together with the nitrogen atom to which they are attached form a saturated 5-, 6- or 7-membered N-containing heterocyclic group which is unsubstituted or substituted; —C(O)R, —C(O)N(R)₂ and —S(O)ₘR wherein R and m are as defined above;
Y is selected from —O—(CH₂)—, —S—(CH₂)ₙ—, and —S(O)(CH₂)ₙ— wherein m is 1 or 2, n is 0 or an integer of 1 to 3, and R² is selected from H or a 5- to 12-membered carbocyclic or heterocyclic group which is unsubstituted or substituted, and a group —NR₃R₄ wherein R₃ and R₄ are as defined above;
Z is selected from (i) halo, —(CH₂)ₛCOOR, —(CH₂)ₛCHO, —(CH₂)ₛCH₂OR, —(CH₂)ₛCONR₃R₄,—(CH₂)ₛCH₂NR₃R₄, —NR₃R₄ and —O(CH₂)ₛNR₃R4 wherein s is 0 or an integer of 1 to 2 and wherein R, R₃ and R₄ are as defined above; (ii) substituted or unsubstituted heteroaryl, (iii) substituted or unsubstituted heterocyclyl, (iv) substituted or unsubstituted aryl, and (v) substituted or unsubstituted C₁-C₆-alkyl; and
W is selected from (i) NR₅R₆, wherein R₅ and R₆ form, together with the N atom to which they are attached, a morpholine ring which is unsubstituted or substituted, (ii) substituted or unsubstituted heteroaryl, (iii) substituted or unsubstituted heterocyclyl, (iv) substituted or unsubstituted aryl, and (v) substituted or unsubstituted C₁-C₆-alkyl;
or a stereoisomer, or a tautomer, or an N-oxide, or a pharmaceutically acceptable salt, or an ester, or a prodrug, or a hydrate, or a solvate thereof.

Organic compounds of this structure, as well as processes of synthesizing organic compounds of this structure are described in U.S. Patent Application Publication No. US 2012/0288492, the entire contents of each of which are incorporated herein by reference.

### Organic Compound Structure V

In some embodiments, the PI3K signal transduction pathway antagonist is an organic compound that binds PI3K having the structure:

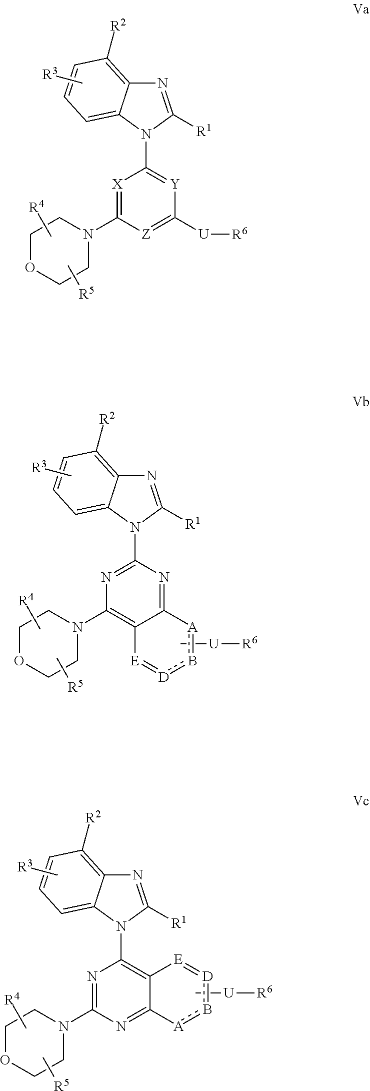

or an enantiomer, a mixture of enantiomers, or a mixture of two or more diastereomers thereof; or a pharmaceutically acceptable salt, solvate, hydrate, or prodrug thereof; wherein:
each R¹ is independently hydrogen, C₁₋₆ alkyl, —S—C₁₋₆ alkyl, —S(O)—C₁₋₆ alkyl, or —SO₂—C₁₋₆ alkyl;
each R² and R³ is independently (a) hydrogen, cyano, halo, ornitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyelyl; or (c) —C(O)R^{1a}, —C(O)OR^{1b}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a}) NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)NR^{1b}R^{1c};
each R⁴ and R⁵ is independently hydrogen or C₁₋₆ alkyl; or R⁴ and R⁵ are linked together to form a bond, C₁₋₆ alkylene, C₁₋₆ heteroalkylene, C₂₋₆ alkenylene, or C₂₋₆ heteroalkenylene;
each R⁶ is independently C₆₋₁₄ aryl, C₇₋₁₅ aralkyl, heteroaryl, or heteroaryl-C₁₋₆ alkyl;
each U is independently a bond, —C(O)—, —C(O)O—, —C(O)NR^{1a}—, —O—, —OC(O)O—, —OC(O)NR^{1a}—, —NR^{1a}—, —NR^{1a}C(O)NR^{1d}—, —NR^{1a}S(O)—, —NR^{1a}S(O)₂—, —NR^{1a}S(O)NR1d-, —NR^{1a}S(O)₂NR^{1d}—, —S—, —S(O)—, or —S(O)₂—;
each X, Y, and Z is independently N or CR⁷, with the proviso that at least two of X, Y, and Z are nitrogen atoms; where R⁷ is hydrogen or C₁₋₆ alkyl; and
each A, B, D, and E is independently a bond, C, O, N, S, NR⁹, CR⁹, or CR⁹R¹⁰, where each R⁹ and R¹⁰ is independently hydrogen, halo, C₁₋₆ alkyl, C₂₋₆ alkenyl, or C₂₋₆ alkynyl; wherein the bonds between A, B, D, and E may be saturated or unsaturated; with the proviso that no more than one of A, B, D, and E are a bond;
each R^{1a}, R^{1b}, R^{1c}, and R^{1d} is independently (i) hydrogen; or (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyelyl;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁵, R⁶, R⁷, R⁹, R¹⁰, R^{1a}, R^{1b}, R^{1c}, or R^{1d} is optionally substituted with one or more, in one embodiment, one, two, three, or four groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q; and (c) —C(O)R^{a}, —C(O)OR^{a}, —C(O)NR^{b}R^{c}, —C(NR) NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)NR^{b}R^{c}, —SR^{a}, —S(O)R^{a}, and —S(O)NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q; or (iii) Rb and Rc together with the N atom to which they are attached form heterocyclyl, optionally substituted with one or more, in one embodiment, one, two, three, or four, substituents Q;
wherein each Q is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, C(O)OR^{e}, —C(O)NR^{f}R^{g}, —C(NR) NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NReC(O)NR^{f}R^{g}, —NR^{e}C(═NR^{h})NR^{f}R^{g}, —NR^{e}S(O)R^{h}, —NR^{e}S(O)NR^{f}R^{g}, —SR^{e}, —S(O)R^{e}, and —S(O)NR^{f}R^{g}, wherein each R^{e}, R^{f}; R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ is aralkyl, heteroaryl, or heterocyclyl; or (iii) R^{f} and R^{g} together with the N atom to which they are attached form heterocyclyl.

Organic compounds of this structure, as well as processes of synthesizing organic compounds of this structure are described in U.S. Patent Application Publication No. US 2011/0009405, the entire contents of each of which are incorporated herein by reference.

### Organic Compound Structure VI

In some embodiments, the PI3K signal transduction pathway antagonist is an organic compound that binds PI3K having the structure:

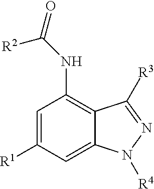

wherein
R¹ is phenyl substituted by one or two substituents independently selected from C₁₋₆ alkyl, —OR⁵, halo, —CN, —COR⁶, CO₂R⁷, —CONR⁸R⁹, —NR¹⁰R¹¹, —NHCOR¹², —SO₂R¹³, —(CH₂)ₘSO₂NR¹⁴R¹⁵, —NHSO₂R₁₆, and 5-membered heteroaryl wherein the 5-membered heteroaryl contains one or two heteroatoms independently selected from oxygen and nitrogen; or pyridinyl optionally substituted by one or two substituents independently selected from C₁₋₆ alkyl, —OR¹⁷, halo, —SO₂R¹⁸, —SO₂NR¹⁹R²⁰, —NHSO₂R²¹ and —NHCOR²⁴;
R² is —(CH₂)ₙ-phenyl optionally substituted by —CN or —NR²²R²³; 5- or 6-membered heteroaryl wherein the 5- or 6-membered heteroaryl contains one or two heteroatoms independently selected from oxygen, nitrogen and sulphur and is optionally substituted by C₁₋₆ alkyl, halo or —(CH₂)_{q}NR²⁵R²⁶; or C₃₋₆ cycloalkyl optionally substituted by phenyl;
R³ is hydrogen or fluoro;
R⁴ is hydrogen or methyl;
R⁷, R¹⁷, R¹⁹, R²⁰, R²², R²³, R²⁷, R²⁸ and R²⁹ are each independently hydrogen or C₁₋₆alkyl;
R⁵ is hydrogen, C₁₋₆ alkyl or —CF₃;
R⁶, R¹², R¹³, R¹⁸, R³³ and R³⁴ are each independently C₁₋₆ alkyl;
R⁸ and R⁹ are each independently hydrogen or C₁₋₆ alkyl, or R⁸ and R⁹, together with the nitrogen atom to which they are attached, are linked to form a 5- or 6-membered heterocyclyl optionally containing an oxygen atom;
R¹⁰ and R¹¹ are each independently hydrogen or C₁₋₆ alkyl, or R¹⁰ and R¹¹, together with the nitrogen atom to which they are attached, are linked to form a 5- or 6-membered heterocyclyl optionally containing an oxygen atom;
R¹⁴ and R¹⁵ are each independently hydrogen, C₁₋₆ alkyl, C₃₋₆ cycloalkyl or —(CH₂)ₚphenyl, or R¹⁴ and R¹⁵, together with the nitrogen atom to which they are attached, are linked to form a 5- or 6-membered heterocyclyl optionally containing an oxygen atom;
R¹⁶ is C₁₋₆ alkyl; or phenyl optionally substituted by C₁₋₆ alkyl;
R²¹ is C₃₋₆ cycloalkyl; C₁₋₆ alkyl optionally substituted by —CF₃; phenyl optionally substituted by one or two substituents independently selected from Ca-6 alkyl, —OR²⁷, —CO2R²⁸ and halo; —(CH₂)ᵤNR³⁵R³⁶; or 5-memberedheteroaryl wherein the 5-membered heteroaryl contains one or two heteroatoms independently selected from oxygen, nitrogen and sulphur and is optionally substituted by one or two substituents independently selected from C₁₋₆ alkyl;
R²⁴ is C₁₋₆ alkyl optionally substituted by —OR²⁹;
R²⁵ and R²⁶, together with the nitrogen atom to which they are attached, are linked to form a 5-, 6- or 7-membered heterocyclyl or a 10-membered bicyclic heterocyclyl wherein the 5-, 6- or 7-membered heterocyclyl or the 10-membered bicyclic heterocyclyl optionally contains an oxygen atom, a sulphur atom or a further nitrogen atom and is optionally substituted by one or two substituents independently selected from C₁₋₆ alkyl, C₃₋₆ cycloalkyl, halo, oxo, phenyl optionally substituted by halo, pyridinyl, —(CH₂)ᵣR³⁰, —(CH)ₛNR³¹R³², —COR³³ and —SO₂R³⁴;
R³⁰ is hydrogen, C₁₋₆ alkyl or —(CH₂), phenyl;
R³¹ and R³², together with the nitrogen atom to which they are attached, are linked to form a 6-membered heterocyclyl optionally containing an oxygen atom;
R³⁵ and R³⁶, together with the nitrogen atom to which they are attached, are linked to form a 5- or 6-membered heterocyclyl wherein the 5- or 6-membered heterocyclyl optionally contains an oxygen atom or a further nitrogen atom and is optionally substituted by one or two substituents independently selected from C₁₋₆ alkyl;
m, n, p, q, r, s and t are each independently 0, 1 or 2; and u is 1 or 2; and salts thereof.

Organic compounds of this structure, as well as processes of synthesizing organic compounds of this structure are described in U.S. Pat. No. 8,163,743, the entire contents of each of which are incorporated herein by reference.

### Organic Compound Structure VII

In some embodiments, the PI3K signal transduction pathway antagonist is an organic compound that binds PI3K having the structure:

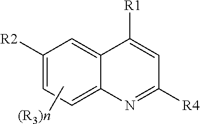

in which
R2 is an optionally substituted ring system selected from a group consisting of: formula (A), (B), (C), (D), (E), (F), (G), (H) and (I):

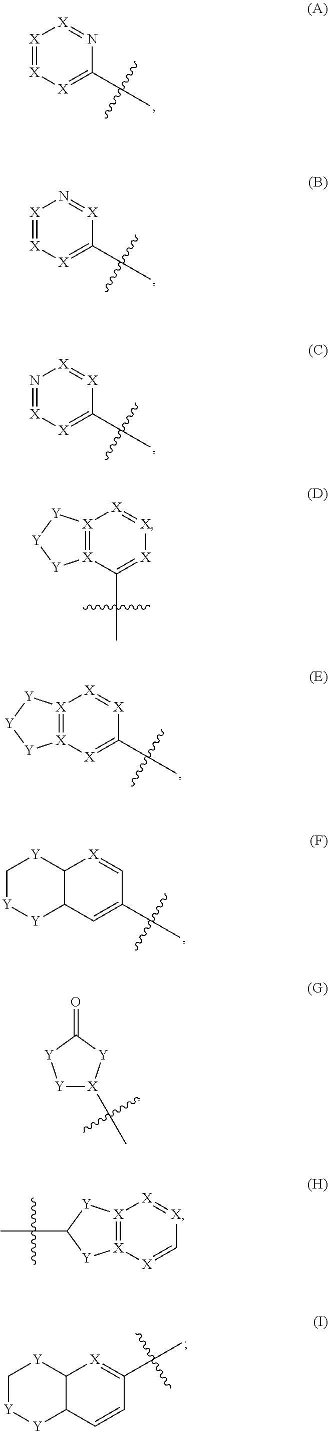

R1 is selected from a group consisting of: heterocycloalkyl, substituted heterocycloalkyl, aryl, substituted aryl, heteroaryl and substituted heteroaryl; each R3 and R4 is independently selected from: hydrogen, halogen, acyl, amino, substituted amino, C₁₋₆ alkyl, substituted C₁₋₆ alkyl, C₃₋₇ cycloalkyl, substituted C₃₋₇ cycloalkyl, C₃₋₇ heterocycloalkyl, substituted C₃₋₇ heterocycloalkyl, alkylcarboxy, aminoalkyl, aryl, substituted aryl, heteroaryl, substituted heteroaryl, arylalkyl, substituted arylalkyl, arylcycloalkyl, substituted arylcycloalkyl, heteroarylalkyl, substituted heteroarylalkyl, cyano, hydroxyl, alkoxy, nitro, acyloxy, and aryloxy;
n is 1-2;
X is C or N; Y is C, O, N or S;
and/or a pharmaceutically acceptable salt thereof,
provided that in each of formula (D) to (I) at least one X or Y is not carbon; further provided that R2 is not quinoline or substituted quinoline.
R3 can be attached to anyone of the four open carbon positions.

Organic compounds of this structure, as well as processes of synthesizing organic compounds of this structure are described in U.S. Pat. No. 8,138,347, the entire contents of each of which are incorporated herein by reference.

### Organic Compound Structure VIII

In some embodiments, the PI3K signal transduction pathway antagonist is an organic compound that binds PI3K having the structure:

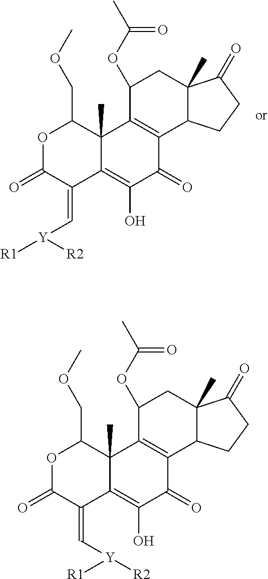

wherein Y is a heteroatom and R1 or R2 are unsaturated alkyl, non-linear alkyl, or substituted alkyl, including a branched alkyl or cyclic alkyl.

Organic compounds of this structure, as well as processes of synthesizing organic compounds of this structure are described in U.S. Pat. No. 7,858,657, the entire contents of each of which are incorporated herein by reference.

### Organic Compound Structure IX

In some embodiments, the PI3K signal transduction pathway antagonist is an organic compound that binds JNK having the structure:

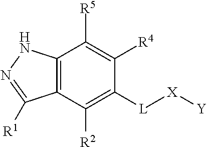

(wherein R¹ is a C₆-C₁₄ aromatic cyclic hydrocarbon group which may be substituted or a 5- to 14-membered aromatic heterocyclic group which may be substituted;
R², R⁴ and R⁵ each independently represent a hydrogen atom, a halogen atom, a hydroxyl group, a cyano group, a nitro group, a carboxyl group, a C₁-C₈ alkyl group which may be substituted, a C₁-C₆ alkoxy group which may be substituted, a C₂-C₇ acyl group which may be substituted, —CO—NR^{2a}R^{2b}, —NR^{2b}CO—R^{2a} or NR^{2a}R^{2b} (wherein R^{2a} and R^{2b} each independently represent a hydrogen atom or a C₁-C₆ alkyl group which may be substituted); L is a single bond, a C₁-C₆ alkylene group which may be substituted, a C₂-C₈ alkenylene group which may be substituted or a C₂-C₈ alkynylene group which may be substituted;
X is a single bond, or a group represented by —NR⁶—, —O—, —CO—, —S—, —SO—, —SO₂—, —CO—NR⁸—V²—, —C(O)O—, —NR⁸—CO—V²—, —NRS—C(O)O—, —NR⁸—S—, —NRS—SO—, —NR⁸—SO₂—V²—, —NR⁸—CO—NR¹⁰—, —NR⁹—CS—NR¹⁰—, —S(O)ₘ—NR¹¹—V²—, —C(NR¹²)—NR¹³—, —OC(O)—, —OC(O)—N—R¹⁴— or —CH₂—NR⁸—COR⁶ (wherein R⁶, R⁸, R⁹, R¹⁰, R¹¹, R¹², R¹³ and R¹⁴ each independently represent a hydrogen atom, a halogen atom, a hydroxyl group, a C₁-C₆ alkyl group which may be substituted, a C₂-C₆ alkenyl group which may be substituted, a C₂-C₆ alkynyl group which may be substituted, a C₁-C₆ alkoxy group which may be substituted, a C₂-C₆ alkenyloxy group which may be substituted, a C₁-C₆ alkylthio group which may be substituted, a C₂-C₆ alkenylthio group which may be substituted, a C₃-C₈ cycloalkyl group which may be substituted, a C₃-C₈ cycloalkenyl group which may be substituted, a 5- to 14-membered non-aromatic heterocyclic group which may be substituted, a C₆-C₁₄ aromatic cyclic hydrocarbon group which may be substituted or a 5- to 14-membered aromatic heterocyclic group which may be substituted; V² is a single bond or a C₁-C₆ alkylene group which may be substituted; and m is 0, 1 or 2); and
Y is a hydrogen atom, a halogen atom, a nitro group, a hydroxyl group, a cyano group, a carboxyl group, a C₁-C₆ alkyl group which may be substituted, a C₂-C₆ alkenyl group which may be substituted, a C₂-C₆ alkynyl group which may be substituted, a C₁-C₆ alkoxy group which may be substituted, a C₃-C₈ cycloalkyl group which may be substituted, a C₃-C₈ cycloalkenyl group which may be substituted, a 5- to 14-membered non-aromatic heterocyclic group which may be substituted, a C₆-C₁₄ aromatic cyclic hydrocarbon group which may be substituted, a 5- to 14-membered aromatic heterocyclic group which may be substituted, an amino group or —W—R¹⁵ (wherein W is —CO— or —SO₂—; and R¹⁵ is a C₁-C₆ alkyl group which may be substituted, a C₆-C₁₄ aromatic cyclic hydrocarbon group which may be substituted, a 5- to 14-membered aromatic heterocyclic group which may be substituted or an amino group)), a salt thereof or a hydrate of them.

Organic compounds of this structure, as well as processes of synthesizing organic compounds of this structure are described in U.S. Pat. No. 7,776,890, the entire contents of each of which are incorporated herein by reference.

### Organic Compound Structure X

In some embodiments, the PI3K signal transduction pathway antagonist is an organic compound that binds JNK having the structure:

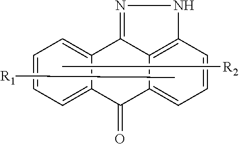

and pharmaceutically acceptable salts thereof, wherein:
R₁ and R₂ are optional substituents that are the same or different and independently represent alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, or a group represented by formula (a), (b), (c) or (d):

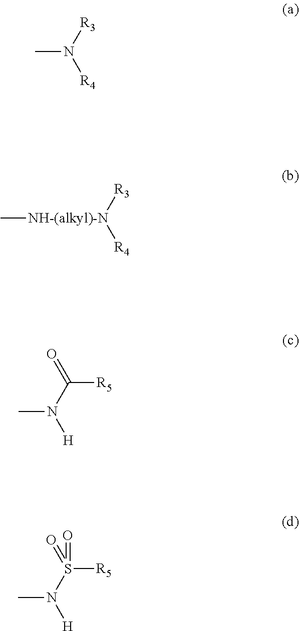

R₃ and R₄ taken together represent alkylidene or a heteroatom-containing alkylidene, or R₃ and R₄ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino); and
R₅ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino.

Organic compounds of this structure, as well as processes of synthesizing organic compounds of this structure are described in U.S. Patent Application Publication No. 2010/0233689, the entire contents of each of which are incorporated herein by reference.

### Organic Compound Structure XI

In some embodiments, the proteasome antagonist is an organic compound having the structure:

A boronic ester of

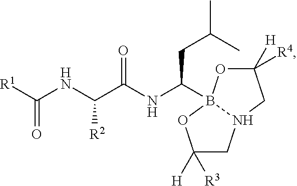

wherein R¹ is 2-(6-phenyl)pyridinyl, R² is (1R)-1-hydroxyethyl, and R³ and R⁴ are H; R¹ is 2-(6-phenyl)pyridinyl, R² is (1R)-1-hydroxyethyl, and R³ and R⁴ are methyl; or R¹ is 2-pyrazinyl, R² is benzyl, and R³ and R⁴ are H. In certain embodiments, R¹ is 2-(6-phenyl)pyridinyl, R² is (1R)-1-hydroxyethyl, and R³ and R⁴ are H. In certain embodiments, R¹ is 2-(6-phenyl)pyridinyl, R² is (1R)-1-hydroxyethyl, and R³ and R⁴ are methyl. In certain embodiments, R¹ is 2-pyrazinyl, R² is benzyl, and R³ and R⁴ are H.

Organic compounds of this structure, as well as processes of synthesizing organic compounds of this structure are described in U.S. Patent Application Publication No. 2012/0270840, the entire contents of each of which are incorporated herein by reference.

### Organic Compound Structure XII

In some embodiments, the proteasome antagonist is an organic compound having the structure:

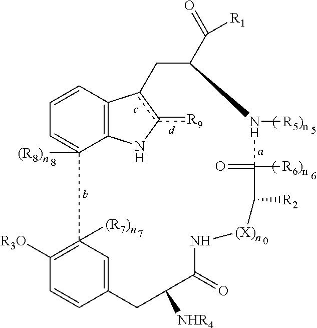

wherein
at least one of the bonds a and b, and only one of the bonds c or d, are present, provided that:
   when the bonds a and b are present simultaneously, then R₉ is H, and n₅=n₆ n₇=n₈=0,
   when the bond a is present, but not the bond b, then n₅=n₆=0, and n₇=n₈=1,
   when the bond b is present, but not the bond a, then n₅=n₆=1, and n₇=n₈=0,
   when the bond c is present, and d is absent, then R₉ is H,
   when the bond d is present, and c is absent, then R₉ is an oxygen atom O,
   n₀ is 0 or 1, and when n₀ is 1, X═CH₂ or X═NCH₂C₆H₅,
R₁ is:
   OH, or a OR₁₀ group in which R₁₀ is a linear or branched alkyl group from 1 to 5 carbon atoms,
   or a group of formula NH—(CH₂)ₙ₁—R₁₁ in which:
      n₁=0, or an integer from 1 to 5,
      R₁₁ is a linear or branched alkyl group from 1 to 5 carbon atoms, an aryl group, possibly substituted, NH₂, or NHR₁₂ in which R₁₂ is a protecting group of amine functions, such as the tertiobutyloxycarbonyl (Boc) group, or the CO—O—CH₂—C₆H₅ (Z) group,
R₂ is:
   H, or a linear or branched alkyl group from 1 to 5 carbon atoms,
   or a group of formula (CH₂)ₙ₂—(CO)ₙ₃—NR₁₃R₁₄, in which:
      n₂ is an integer from 1 to 5,
      n₃=0 or 1,
      R₁₃ and R₁₄, independently from one another, are:
         H,
         or a protecting group of amine functions, such as Boc, or Z,
         or a group of formula C(═NH)NHR₁₅ in which R₁₅ is H or a protecting group of amine functions, such as Boc, or Z, mentioned above,
   or a side chain from proteogenic amino acids,
R₃ is H, or a linear or branched alkyl group from 1 to 5 carbon atoms, optionally substituted with an aryl group,
R₄ is H, or a protecting group of amine functions, such as Boc, or Z,
R₅ is H, or a protecting group of amine functions, such as Hoc, or Z,
R₆ is a OR₁₆ group in which R₁₆ is a linear or branched alkyl group from 1 to 5 carbon atoms,
R₇ and R₈, independently from one another, are H, or a halogen atom, such as Br, I, or Cl.

Organic compounds of this structure, as well as processes of synthesizing organic compounds of this structure are described in U.S. Pat. No. 7,919,468, the entire contents of each of which are incorporated herein by reference.

Ester derivatives of compounds may be generated from a carboxylic acid group in accordance with the present invention using standard esterification reactions and methods readily available and known to those having ordinary skill in the art of chemical synthesis. Ester derivatives may serve as pro-drugs that can be converted into compounds of the invention by serum esterases.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

Compounds used in the methods of the present invention may be prepared by techniques well know in organic synthesis and familiar to a practitioner ordinarily skilled in the art. However, these may not be the only means by which to synthesize or obtain the desired compounds.

Compounds used in the methods of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In some embodiments, a compound may be in a salt form. As used herein, a “salt” is a salt of the instant compound which has been modified by making acid or base salts of the compounds. In the case of the use of compounds of the invention for treatment of cancer, the salt is pharmaceutically acceptable. Examples of pharmaceutically acceptable salts include, but are not limited to, mineral or organic acid salts of basic residues such as amines. The term “pharmaceutically acceptable salt” in this respect, refers to the relatively non-toxic, inorganic and organic base addition salts of compounds of the invention. These salts can be prepared in situ during the final isolation and purification of a compound, or by separately reacting a purified compound in its free acid form with a suitable organic or inorganic base, and isolating the salt thus formed.

### Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of target gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester intemucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). An antisense oligonucleotide also can be designed to block translation of mRNA by preventing the transcript from binding to ribosomes.

Precise complementarity is not required for successful complex formation between an antisense oligonucleotide and the complementary sequence of a target polynucleotide. Antisense oligonucleotides which comprise, for example, 1, 2, 3, 4, or 5 or more stretches of contiguous nucleotides which are precisely complementary to a target polynucleotide, each separated by a stretch of contiguous nucleotides which are not complementary to adjacent nucleotides, can provide sufficient targeting specificity for a target mRNA. Preferably, each stretch of complementary contiguous nucleotides is at least 4, 5, 6, 7, 8, 9, 10, 11, 12, 13, 14, 15, 16, 17, 18, 19, 20, 21, 22, 23, 24, 25, or more nucleotides in length. Noncomplementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular target polynucleotide sequence. Antisense oligonucleotides can be modified without affecting their ability to hybridize to a target polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art.

### Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to rnRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease target gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional teminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

### RNA Interference

Some embodiments the invention relate to an interfering RNA (RNAi) molecule. RNAi involves mRNA degradation. The use of RNAi has been described in Fire et al., 1998, Carthew et al., 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to a siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

### Use of RNAi

RNAi is a powerful tool for in vitro and in viva studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Multiple mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

Another mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell at al., 2006, Drug Discovery Today 11: 975-982). Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

Another mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects. The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

Isolated RNA molecules can mediate RNAi. That is, the isolated RNA molecules of the present invention mediate degradation or block expression of mRNA that is the transcriptional product of the gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

In some embodiments, an RNAi molecule of the invention is introduced into a mammalian cell in an amount sufficient to attenuate target gene expression in a sequence specific manner. The RNAi molecules of the invention can be introduced into the cell directly, or can be complexed with cationic lipids, packaged within liposomes, or otherwise delivered to the cell. In certain embodiments the RNAi molecule can be a synthetic RNAi molecule, including RNAi molecules incorporating modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications. In some embodiments, an RNAi molecule of the invention contains modified nucleotides that increase the stability or half-life of the RNAi molecule in vivo and/or in vitro. Alternatively, the RNAi molecule can comprise one or more aptamers, which interact(s) with a target of interest to form an aptamer:target complex. The aptamer can be at the 5′ or the 3′ end of the RNAi molecule. Aptamers can be developed through the SELEX screening process and chemically synthesized. An aptamer is generally chosen to preferentially bind to a target. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23.

### Administration

“Administering” the antagonists described herein can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject. In embodiments, administration is effected by injection or via a catheter.

Injectable drug delivery systems may be employed in the methods described herein include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, the term “effective amount” refers to the quantity of a component that is sufficient to treat a subject without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of subject being treated, the duration of the treatment, the nature of concurrent therapy (if any), and the specific formulations employed and the structure of the compounds or its derivatives.

The compounds used in the methods of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes are also a pharmaceutically acceptable carrier. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, vol 40 (Gilbert S. Banker, Christopher T. Rhodes, Eds.). All of the aforementioned publications are incorporated by reference herein.

The dosage of a compound of the invention administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the invention may comprise a compound alone, or mixtures of a compound with additional compounds used to treat cancer. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

A compound of the invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297, issued Sep. 2, 1975.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

A compound of the invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

A compound of the invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, a compound may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain a compound of the invention and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, a compound may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

A compound may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions thereof of the invention can be coated onto stents for temporary or permanent implantation into the cardiovascular system of a subject.

### Kits of the Invention

The materials for use in the methods of the present invention are suited for preparation of kits produced in accordance with well known procedures. The invention thus provides embodiments and kits comprising agents, which may include gene-specific or gene-selective probes and/or primers, for quantitating the expression of the disclosed genes, such as for predicting prognostic outcome or response to treatment. Such kits may optionally contain reagents for the extraction of polypeptides or nucleic acids from biological samples. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). In some embodiments, the kits comprise one or more reagents such as one or more antibodies for detecting the amount of a protein. In some embodiments, an antibody may be specific for a phosphorylated form of a protein. Mathematical algorithms used to estimate or quantify prognostic or predictive information are also properly potential components of kits.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### Methods Summary

Targeted expression in the adult hindgut: Multigenic combinations were targeted to the adult hindgut using the hindgut specific gal4 line byn-gal4 (V. Hartenstein) and tub-gal80^{ts} (Bloomington). Crosses were kept at 16° C. to keep the transgenes silent during development and adult females were transferred to 29° C. to induce transgene expression. In addition, GFP and Dcr2 (Dicer 2) were co-expressed to mark the hindgut cells and to facilitate RNAi-mediated knock-down, respectively.

Immunohistochemistry: Primary antibodies used in this study were rabbit anti-Laminin (1:500, Abcam), mouse anti-MMP1 (1:100, DSHB), rabbit anti-Src-pY418 (1:100, Invitrogen), mouse anti-BRDU (1:10, BD Biosciences) and rabbit anti-cleaved Caspase 3 (1:100, Cell Signaling). Alexa-568 or 633 conjugated goat anti-mouse and anti-rabbit antibodies were used as secondary antibodies (1:1000, Invitrogen). Muscle labeling was performed with Alexa-568 conjugated Phalloidin (1:200, Invitrogen) SA-β-gal staining was performed using a kit from Cell Signaling (cat #9860).

Western Blot Analysis: Primary antibodies used were rabbit anti-Drosophila phospho-AKT (S505) (1:1000, Cell Signaling), rabbit anti-pan-AKT (1:1000, Cell Signaling), rabbit anti-phospho-4EBP (T37/46) (1:1000, Cell Signaling), rabbit anti-human phospho-AKT (Ser473) (1:1000, Cell Signaling), mouse anti-α-actin (1:1000, Cell Signaling) and mouse anti-Syntaxin (1:1000, DSHB). HRP-conjugated anti-mouse and anti-rabbit antibodies were used as secondary antibodies (1:5000, Cell Signaling). For signal detection, Immobilon Chemiluminescent HRP Substrate (Millipore) was used.

Compound Feeding: Compound treated food was made by diluting compound stocks (made in 100% DMSO) in semi-defined Drosophila medium to achieve a final concentration of 0.5% DMSO (empirically determined as the non-toxic DMSO dose in semi-defined Drosophila medium). Adult flies were kept on compound treated medium in standard Drosophila vials for 7 days (30 flies/vial, 1 ml food/vial) and provided with fresh compound food every other day.

Cell culture: Parental DLD-1 and HCT-116 cell lines (ATCC) and their PI3K wildtype derivatives (DLD-1 WT and HCT116 WT) (38) that were obtained from Dr. Bert Vogelstein's laboratory were maintained using DMEM with 10% FBS. All experiments were done under low serum (2.5% FBS) conditions (38).

### Example 2

### Methods

Fly Strains: Multigenic combinations were generated by standard genetic crosses using the following fly lines (chromosome locations and sources in parentheses): UAS-ras^{G12V} (2^{nd}, G. Halder), UAS-egfr (2^{nd}, Bloomington), UAS-p53^{RNAi} (2^{nd}, VDRC), UAS-pten^{RNAi} (3^{rd}, VDRC), UAS-apc1^{RNAi} (3^{rd}, VDRC), UAS-apc2^{RNAi} (X, VDRC) and UAS-dSmad4^{RNAi} (3^{rd}, VDRC). Adult females used in this study were generated by crossing flies carrying these multigenic combinations to UAS-dcr2; +; byn-gal4 UAS-GFP tub-gal80^{ts}/S-T at 16° C.

Immunohistochemistry: Hindguts dissected in ice-cold PBS were fixed with 4% paraformaldahyde, 0.3% Triton X in PBS (ice-cold) for 15 minutes at room temperature and rinsed 3 times in PBS, followed by a 15 minute PBS wash. Tissues then were blocked in 0.1% Triton X, 1% normal goat serum in PBS at room temperature for 1 hour, incubated with primary antibody at 4° C. overnight, rinsed 3 times in PBS, blocked for 1 hour and incubated in secondary antibody for 2 hours at room temperature. Hindguts were mounted in Vectashield with DAPI (Vector Laboratories). Primary and secondary antibodies were diluted in block solution.

Western Blot Analysis: Tissue lysates for western analysis were made by grinding 10-20 hindguts in lysis buffer (50 mM Tris-HCl, 150 mM NaCl, 1 mM EDTA, 1% NP-40) supplemented with protease and phosphatase inhibitor cocktails (Sigma). Lysates were boiled for 10 minutes in SDS Sample buffer and reducing agent, resolved on SDS-Page and transferred using standard protocols. Cell lysates are prepared similarly. Protein concentrations were determined using BioRad Protein Assay Dye Reagent following manufacturer's instructions.

MTT assay: Cells were grown in sterile 25 cm² flasks to 80% confluency, trypsinized, resuspended in 100 mL DMEM with 2.5% FBS and seeded in 96-well plates in equal numbers. After 24 hours, cells were treated with compounds diluted in DMEM with 2.5% FBS as indicated for 1, 2 or 3 days. MTT assay was performed by replacing compound containing medium with fresh DMEM with 2.5% FBS and 10 mg/ml MTT reagent (Thiazoyl Blue tetrazolium Bromide, Fisher Scientific) in PBS at a ratio of 5:1 (120 μl/well; 96-well plates). Cells are incubated at 37° C. for 3 hours, then the medium is removed and cells are incubated in MTT solvent (4 mM HCl, 0.1% NP40 in isopropanol) on a shaker. The amount of MTT formazan in the resulting solution is spectrophotometrically measured at 590 nM (and a reference filter of 620 nM). Adjusted absorbance value for each well is calculated using the following formula: (OD₅₉₀SAMPLE-OD₅₉₀BLANK)−(OD₆₂₀SAMPLE-OD₆₂₀BLANK). Each experiment is done in quadruple and relative viability is expressed as fold change in adjusted absorbance compared to untreated and DMSO treated controls in each plate.

BRDU incorporation assay: To label proliferating cells, adults were fed 5 mg/ml BRDU in semi-defined Drosophila medium for 7 days; animals were provided with fresh BRDU-food daily. Dissected guts were processed for antibody staining as described above with the addition of a DNAse treatment step (200 U/ml) at 37° C. for 1 hr before incubation with primary antibody.

Compound Feeding: Compounds used in this study were (stock concentrations and sources in parentheses) AZD6244 (200 mM, Selleck Chemicals), SL327 (200 mM, Tocris), GW5074 (100 mM, Tocris), Sorafenib (200 mM, LC Labs), LY294002 (200 mM, LC Labs), Rapamycin (200 mM, LC Labs), BEZ235 (20 mM, LC Labs), Dasatinib (200 mM, LC Labs), SP600125 (200 mM, LC Labs), Bortezomib (200 mM, LC Labs), Cisplatin (200 mM, Sigma), LBH589 (200 mM, LC Labs), Wortmannin (200 mM, LC Labs), PI-103-HCl (100 mM, Tocris), Everolimus (100 mM, LC Labs) and Enzastaurin (50 mM, LC Labs).

Imaging and Scoring: Fluorescence images were taken using a Leica TCS SPE confocal microscope and processed using the Leica LAS-AF software. Scoring and imaging of SA-β-gal staining was performed using an Olympus BX41 microscope and a Nikon DS-Ri1 camera. Distant migration was imaged and scored using a Leica MZ16F dissecting scope with a GFP filter under 10× magnification.

Statistical Analysis: Statistical significance for the distant migration assay was calculated using Fisher's exact test as this test allows the analysis of contingency tables where sample sizes are relatively small. Use of a 4×2 contingency table allowed pairwise comparisons of the distant migration phenotype, which has four phenotypic categories, between different genotypes and/or treatment conditions. Each experiment was performed in duplicate (n=25-30 for each replicate) and multiple times. Error bars indicate standard error of the mean.

### Example 3

### Multigenic Models of Colorectal Tumors

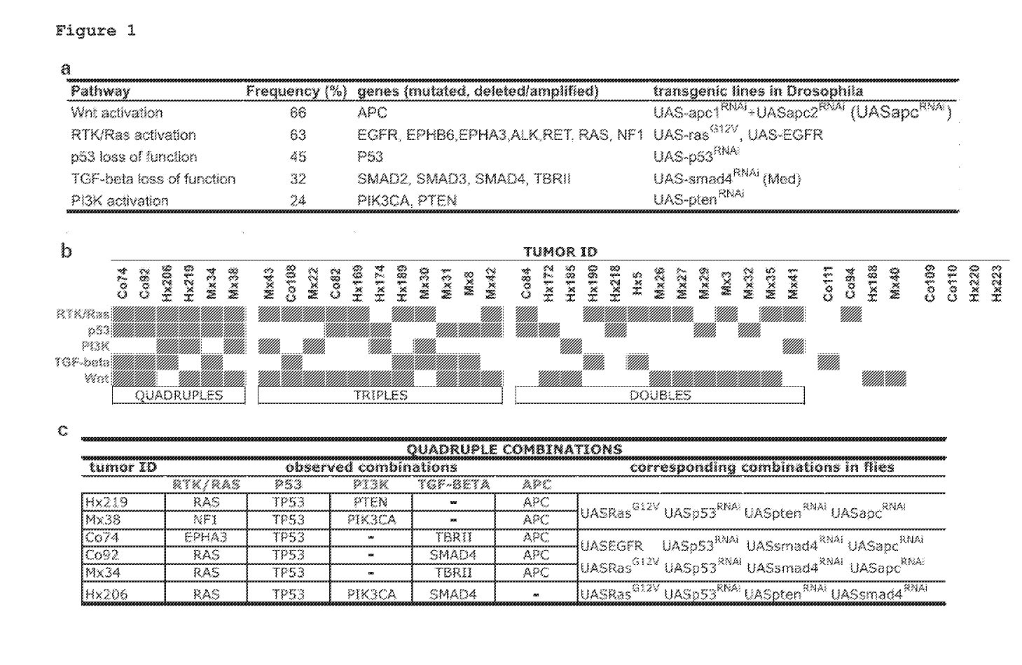

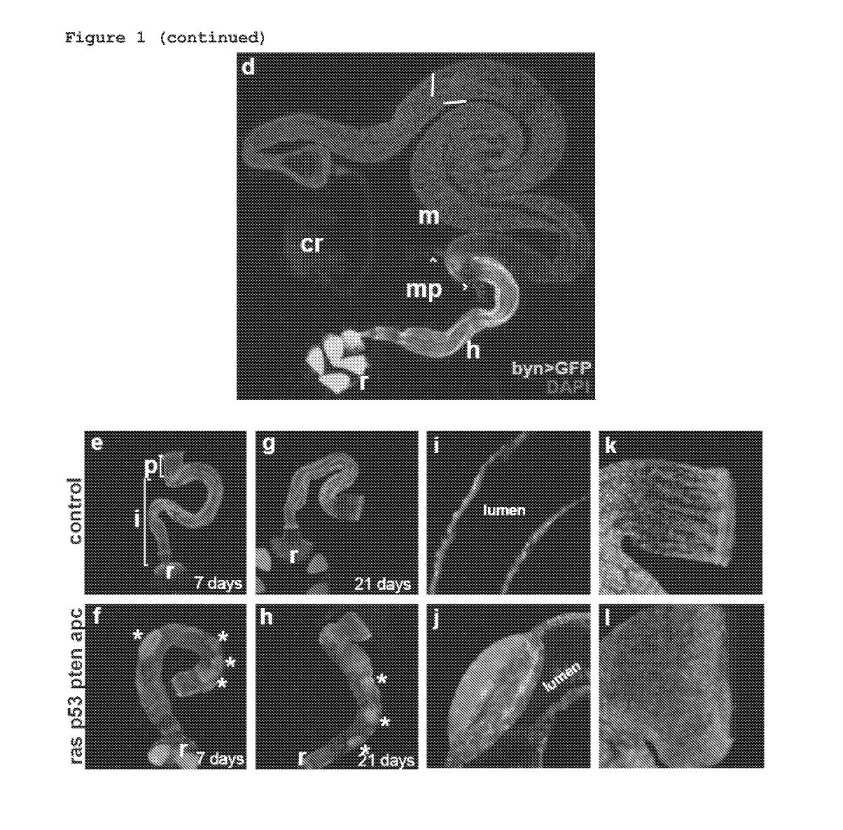

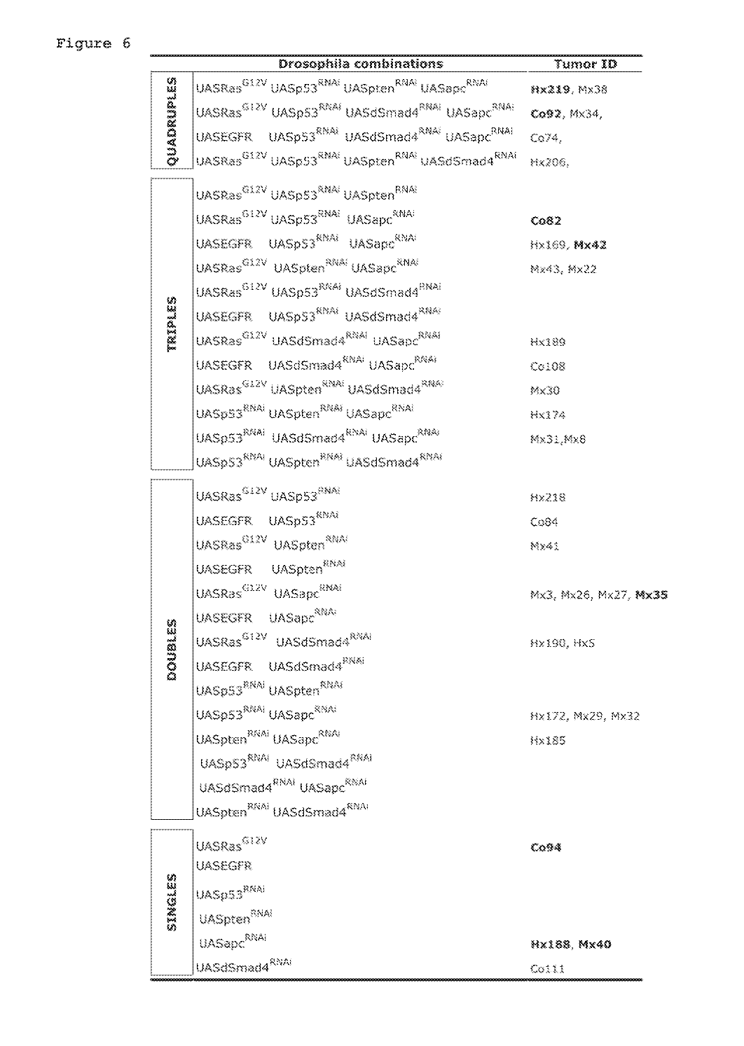

Mutational profiling data available from colon tumors (7-9) indicated that five pathways are most commonly misregulated in colon cancer: Wnt, Receptor Tyrosine Kinase (RTK)/Ras, p53, TGF-β and PI3K/Akt (FIG. 1a). To more accurately model specific human tumors in Drosophila, individual patient tumors that carried mutations in any combination of two, three or four of these pathways were identified (FIG. 1b); no tumors were reported to contain mutations in all five pathways. Transgenic lines were then used to represent the most frequently mutated genes in each pathway (FIG. 1a), generating the most common double, triple and quadruple combinations reported for human colon tumors. The four quadruple combinations that were generated and the mutation complement of the corresponding human colon tumors are shown in FIG. 1c. FIG. 6 contains a complete list of the 30 tumor combinations we generated in flies; of note, loss of apc was modeled by knockdown of both apc1 plus apc2.

Hallmarks of cancer include hyperproliferation, disruption of the normal tissue architecture, evasion of apoptosis and oncogene-induced senescence, migration, and metastasis (11). To determine which aspects of tumorigenesis were recapitulated by the multigenic combinations, the appropriate transgenes were targeted specifically to the adult Drosophila hindgut epithelium—the functional equivalent of the mammalian colon (12,13) (FIG. 1d)-using the temperature sensitive Gal4/Gal80^{ts} system and the byn promoter to control the timing and location of transgene expression (14,15). The adult hindgut is a single-layer epithelium divided into three main sections along its anterior-posterior axis (12,13) (FIG. 1e): (i) the pylorus is the anterior-most region of the hindgut that controls the passage of gut contents from the midgut to the hindgut, (ii) the ileum contains the differentiated enterocytes, and (iii) the rectum sits most posteriorly.

The analysis was focused to one of the most frequently observed quadruple combinations in human tumors: ras^{G12V} p53^{RNAi} pten^{RNAi} apc^{RNAi}. When induced, animals carrying this combination overexpressed ras^{G12V}, an oncogenic form of ras, and targeted tumor suppresors p53, pten and apc by RNAi specifically in the adult hindgut. This four-hit model of colon cancer was found to recapitulate key aspects of human cancer: overproliferation and disruption of the epithelial architecture were observed, as well as evasion of apoptosis and oncogene-induced senescence. Furthermore, hindgut epithelial cells carrying quadruple combinations lost their epithelial characteristics, extending membrane processes towards the basement membrane, delaminating from the epithelium, and migrating away. These changes were associated with elevated, membrane localized pSrc, activation of MMP1 expression, and degradation of the basement membrane.

The fate of delaminated cells subsequent to leaving the hindgut epithelium was also investigated. Numerous disseminated foci were observed throughout the body including the head, legs, and below the epidermis of the abdomen. Large numbers of disseminated foci of varying sizes were also evident within the abdominal cavity. Many of the foci were composed of multiple cells. This dissemination phenotype was quantified by categorizing the animals based on the number disseminated foci in the abdominal cavity over time (FIG. 1a).

A strong dissemination phenotype was evident starting at 7 days after induction. Overall, these findings indicate migrating cells were able to reach distant sites within the body as far as the head and the legs and survive in these foreign environments, recapitulating key early aspects of metastasis.

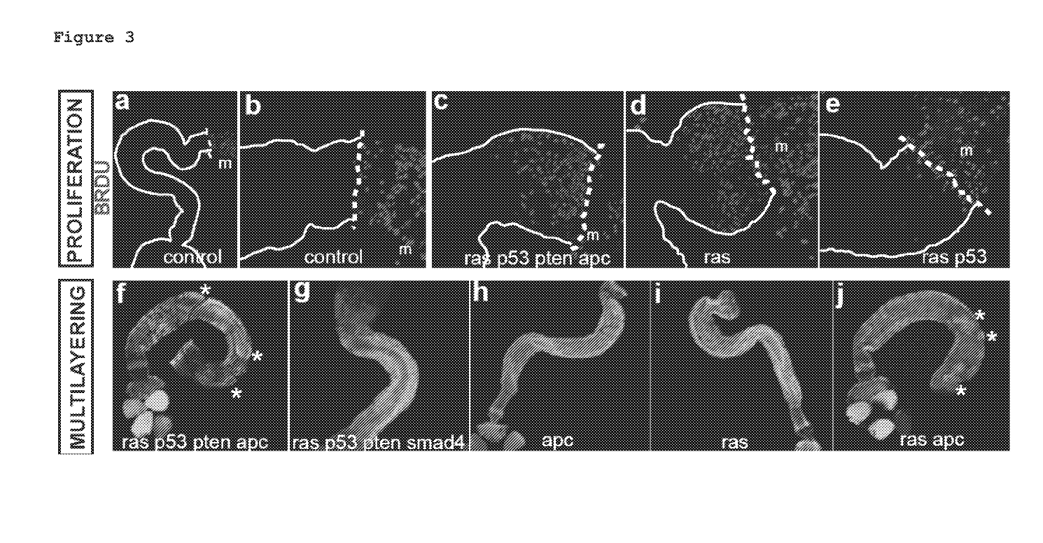

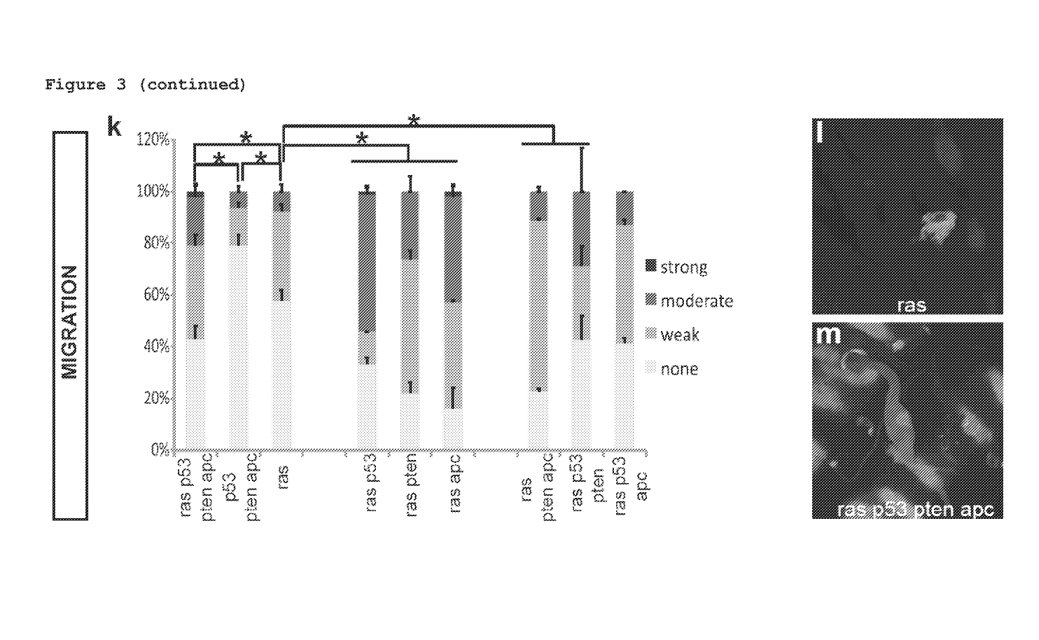

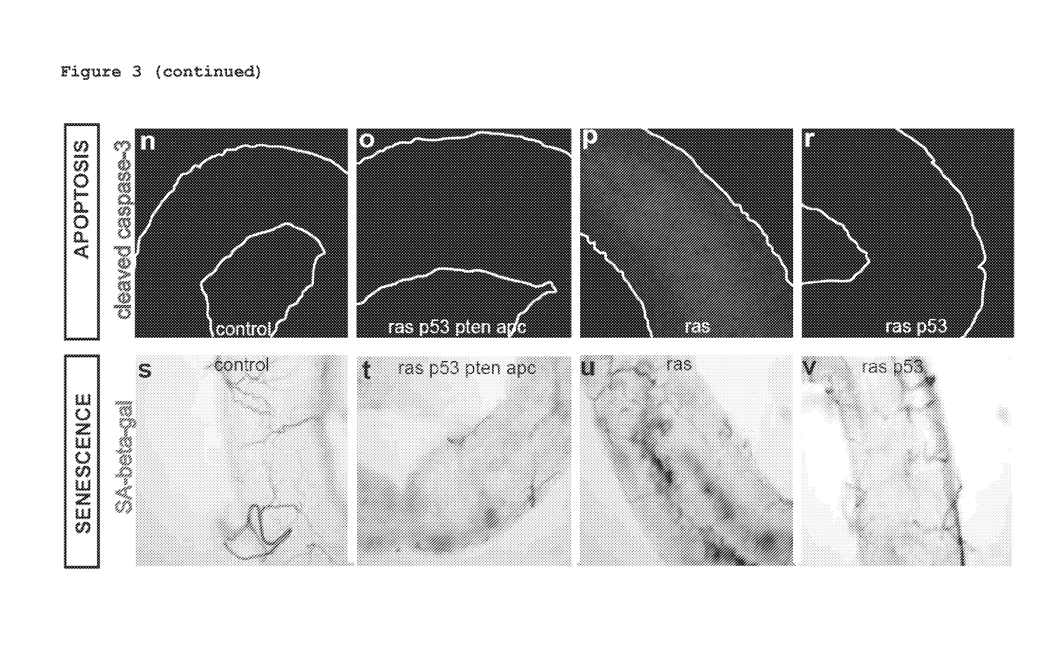

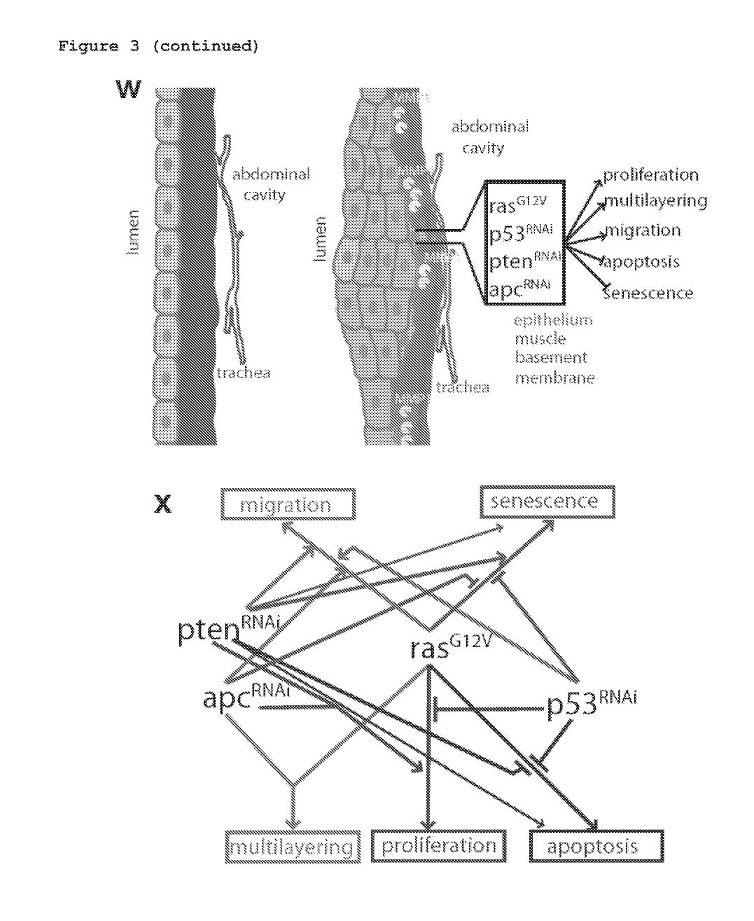

The quadruple combinations were targeted to the adult hindgut along with Dicer2 and GFP. One week after induction two phenotypes became apparent: regions of multilayered epithelia formed as bulges at discrete points along the hindgut (FIG. 1e-j), and the pylorus was expanded (FIG. 1k,l) likely due to hyperproliferation. Interestingly, while expansion of the pylorus was observed in response to all four quadruples, the multilayering was not observed with ras^{G12V} p53^{RNAi} pten^{RNAi} smad4^{RNAi} (FIG. 3g), indicating that reduced apc is a required component.

### Example 4

### Transformed Cells Displayed Migratory Behavior

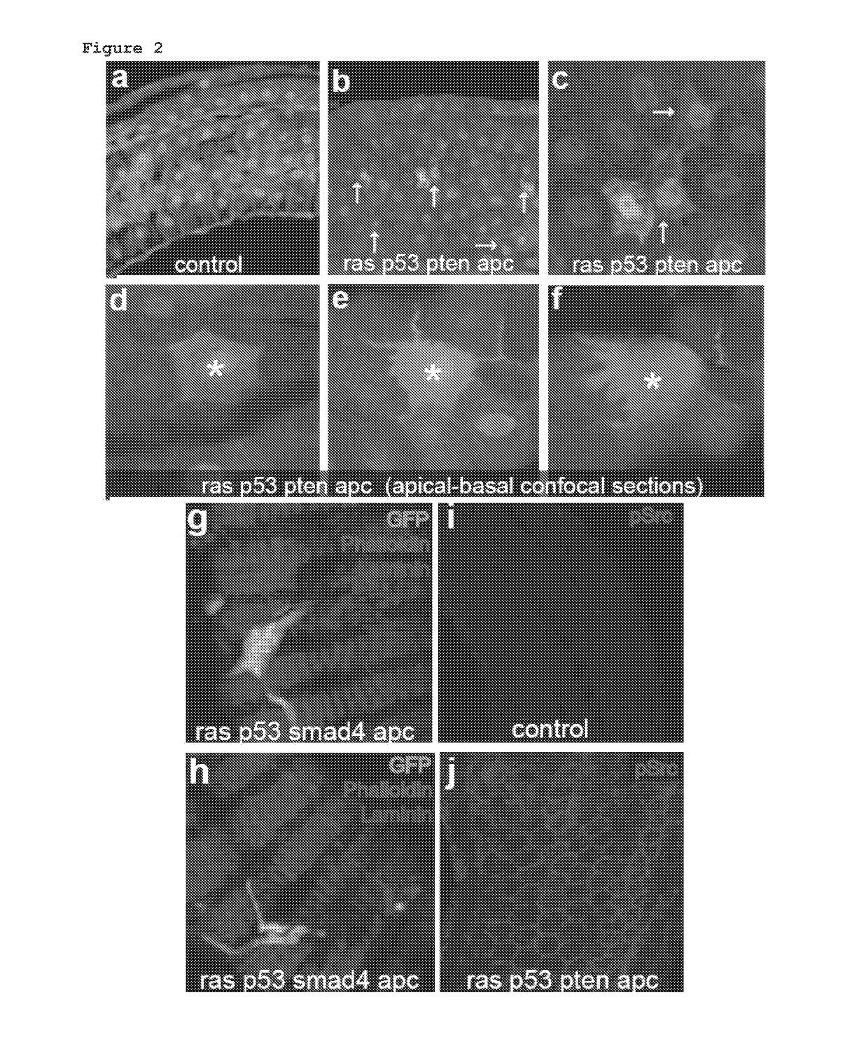

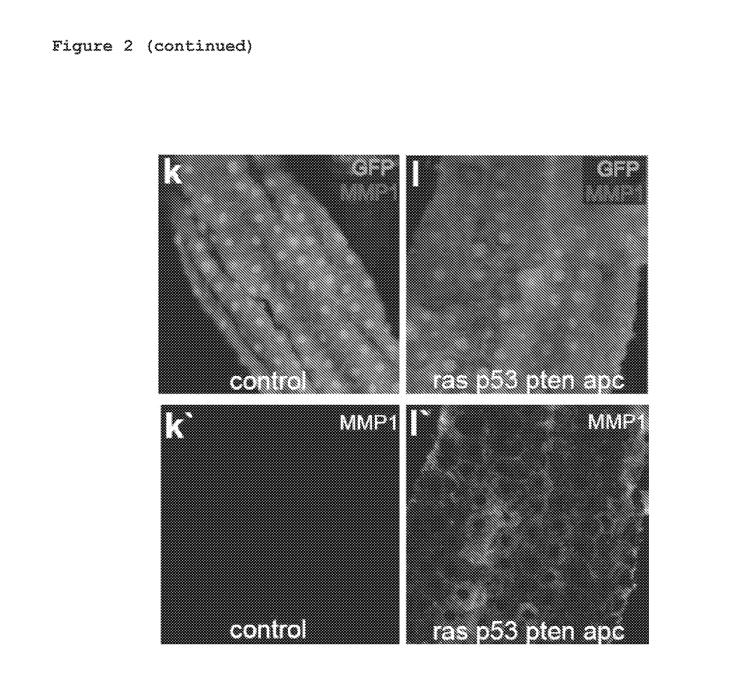

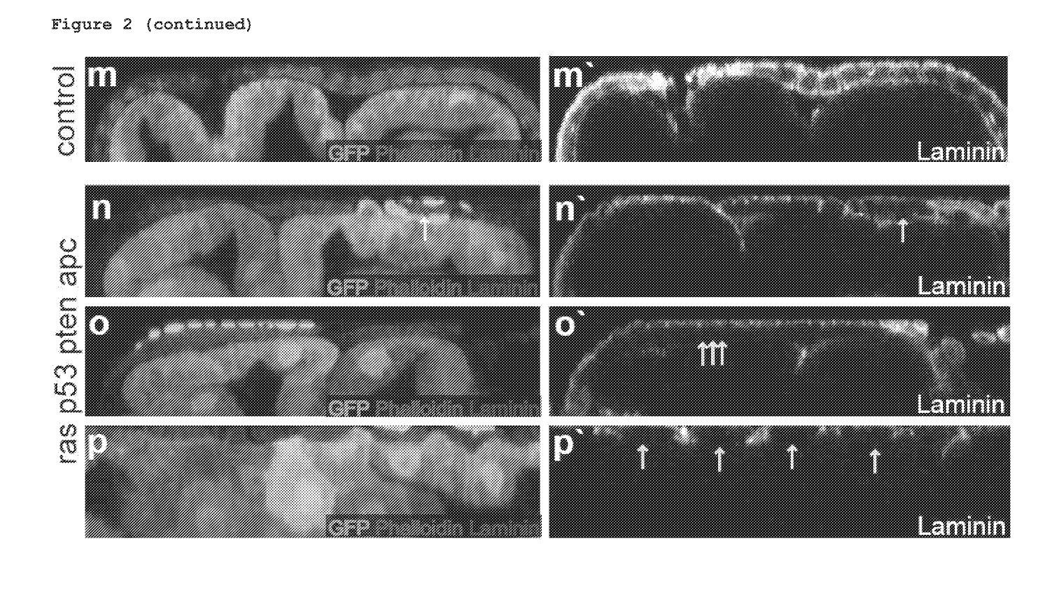

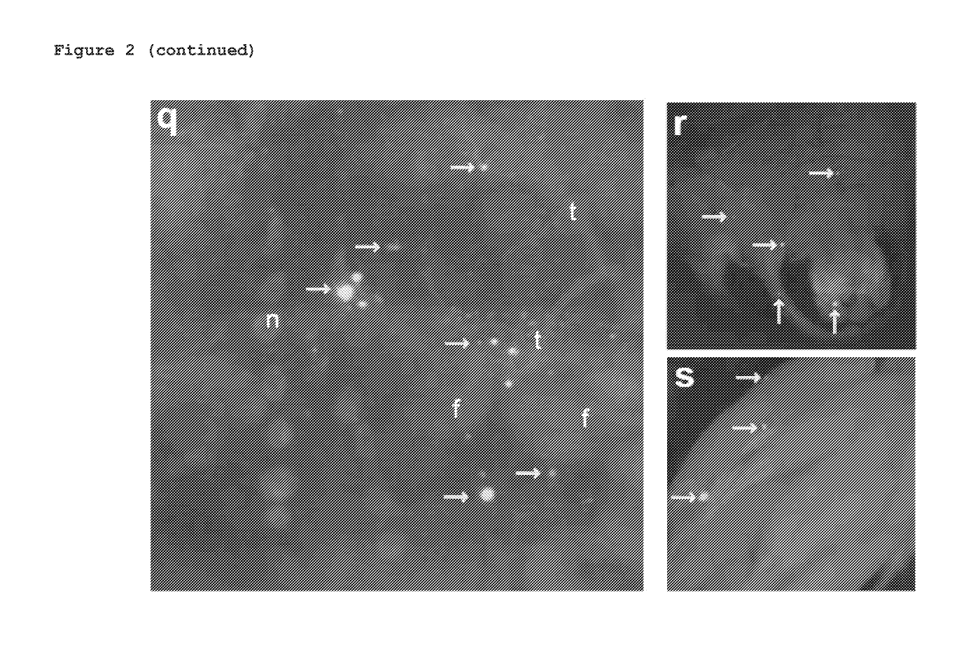

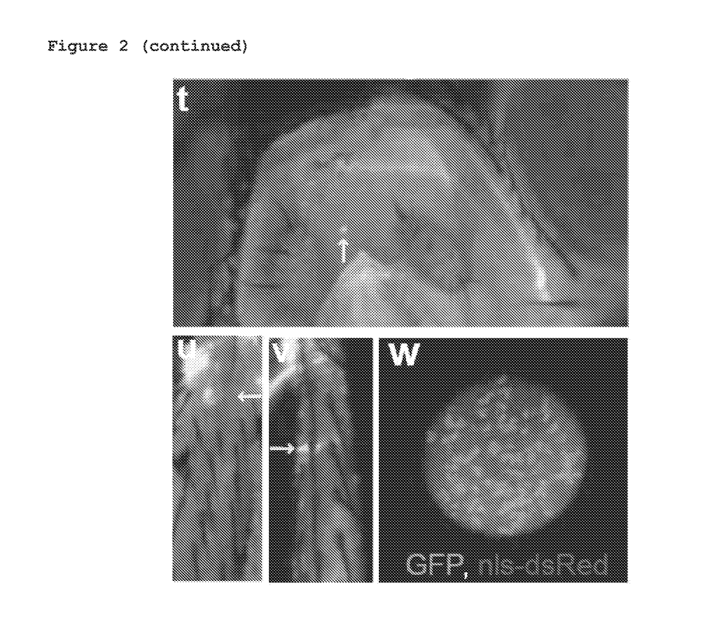

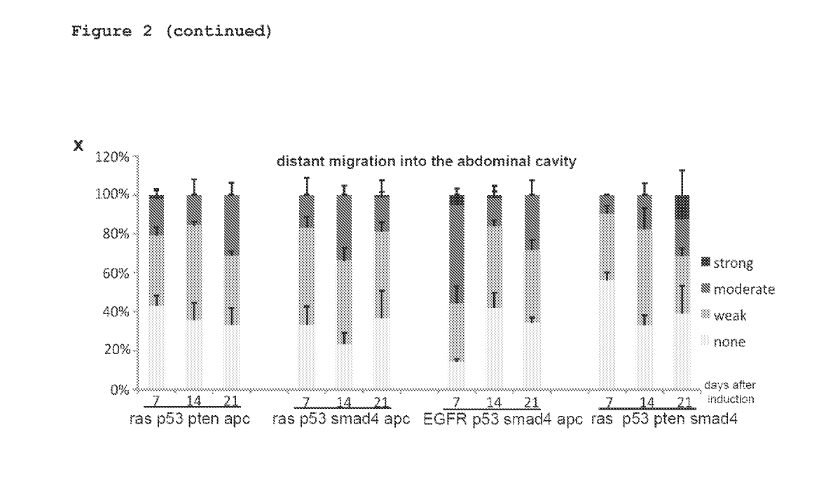

A closer inspection of the hindgut epithelium in quadruple combination animals revealed numerous cells that had lost their characteristic epithelial shape and assumed a more mesenchymal appearance; these cells extended processes towards the basement membrane and the surrounding muscle layer (FIGS. 2a-f). Further, many epithelial cells left the hindgut epithelium to migrate on top of the surrounding muscle layer (FIGS. 2g,h). These migrating cells commonly enwrapped tracheal branches, a tubular network that provides oxygen to Drosophila tissues.

Src is a key regulator of cell migration that is up-regulated in many advanced tumor types (16). Control guts exhibited low uniform phospho-Src (pSrc) levels throughout the cytoplasm with weak membrane localization in some cells. By contrast, hindguts carrying quadruple combinations displayed elevated levels of pSrc that included strong membrane localization (FIG. 2i,j). During the process of migration and metastasis, tumor cells also typically secrete matrix metalloproteases (MMPs) to degrade the basement membrane during metastasis (17). While control hindguts did not show detectable MMP1 expression, hindguts carrying quadruple combinations showed strong but non-uniform MMP1 expression throughout the epithelium (FIGS. 2k,l). They exhibited weak, patchy and absent staining of the basement membrane component Laminin particularly in the region between the epithelium and the surrounding muscle, indicating that the integrity of the basement membrane was compromised (FIG. 2m-p).

In summary, hindgut epithelial cells carrying quadruple combinations lost their epithelial characteristics, extending membrane processes towards the basement membrane, delaminating from the epithelium, and migrating away. These changes were associated with elevated, membrane localized pSrc, activation of MMP1 expression, and degradation of the basement membrane.

### Example 5

### Transformed Cells Migrated to Distant Sites

Next, the fate of delaminated cells subsequent to leaving the hindgut epithelium was investigated. Animals carrying quadruple combinations displayed numerous GFP-positive foci throughout their body including the head, legs, and below the epidermis of the abdomen (FIGS. 2r,t-v). Large numbers of GFP-positive foci of varying sizes were also evident within the abdominal cavity, most of which were loosely associated with tracheal branches (FIG. 2q). Many foci were found along the abdominal body wall near the heart tube, but hindgut cells were also occasionally observed embedded in the fat body and ovaries (FIG. 2q,s). Many of the foci were composed of multiple cells (FIG. 2w). These findings indicate migrating cells were able to reach distant sites within the body as far as the head and the legs and survive in these foreign environments, recapitulating key early aspects of metastasis.

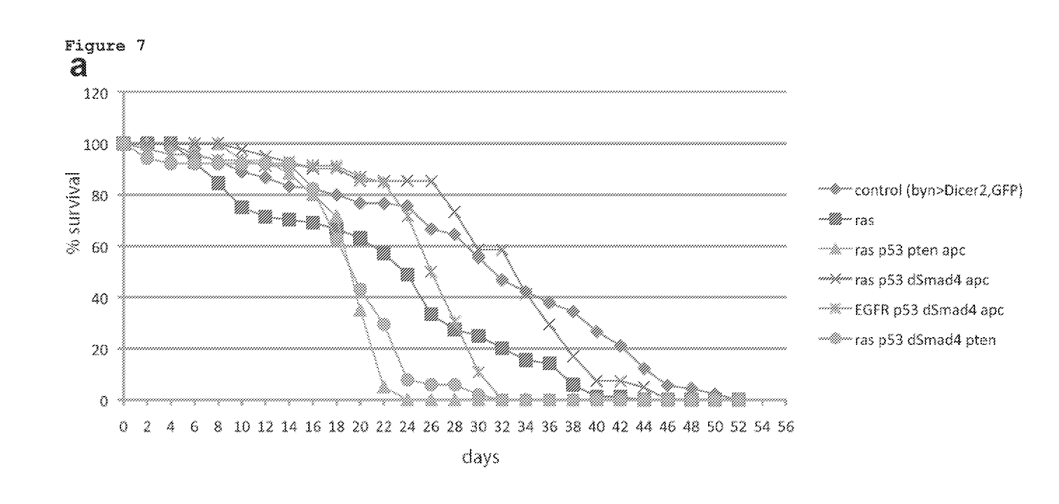

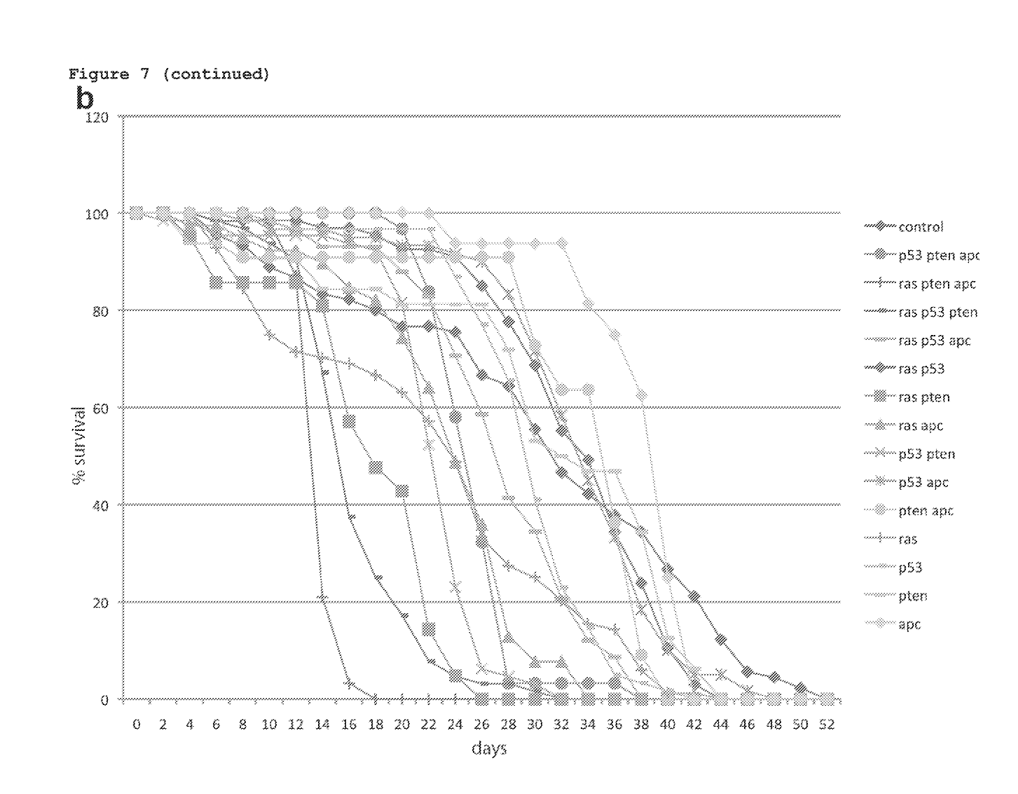

This dissemination phenotype was quantified by categorizing the animals based on the number of GFP-positive foci in the abdominal cavity over time (FIG. 2x). A strong dissemination phenotype was evident with all four combinations starting at 7 days after induction, with egfr p53^{RNAi} smad4^{RNAi} apcR^{RNAi} displaying the strongest phenotype. While the migration phenotype was progressively stronger and more penetrant over time in some combinations, in others it was not. This likely reflects the consequences of constant transgene expression throughout the hindgut, which compromised tissue integrity over time and interfered with the migration process. These combinations led to increased host mortality (FIG. 7) and hindguts with the most severe phenotypes may have been removed from the pool.

### Example 6

### Proliferation, Multilayering, and Migration are Regulated by Complex Gene Combinations

Though the Drosophila hindgut epithelium is normally quiescent, hindgut stem cells can be stimulated to proliferate in response to tissue damage (12). It was reasoned herein that expansion of the pylorus region in response to the quadruple combinations could be due to proliferation of the normally quiescent stem cells. Consistent with previous work (12), BRDU-positive cells were observed in control hindguts (FIG. 3a,b) of animals labeled for 7 days. However, large numbers of BRDU-positive cells were observed in the pylorus of ras^{G12V} p53^{RNAi} pten^{RNAi} apc^{RNAi} hindguts (FIG. 3c), consistent with proliferation.

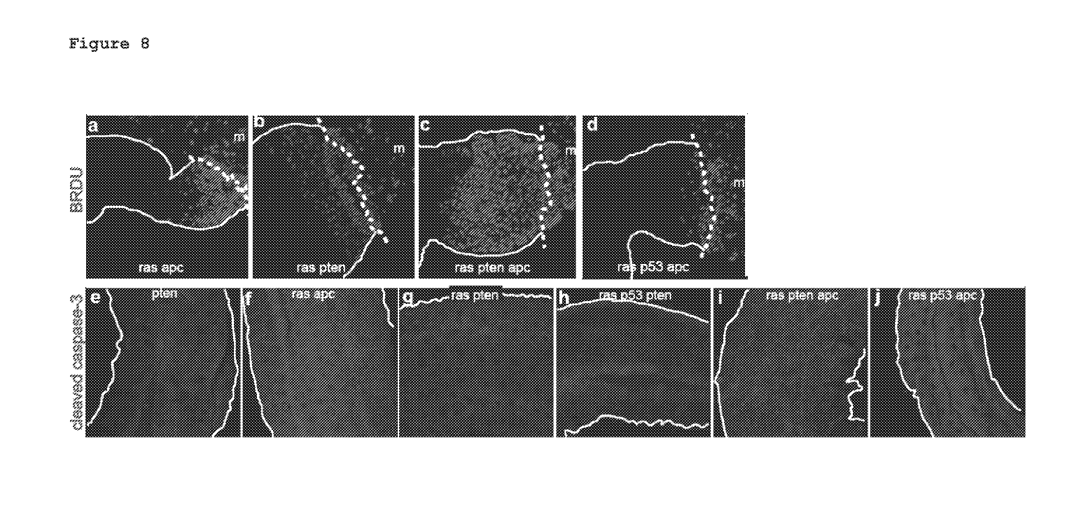

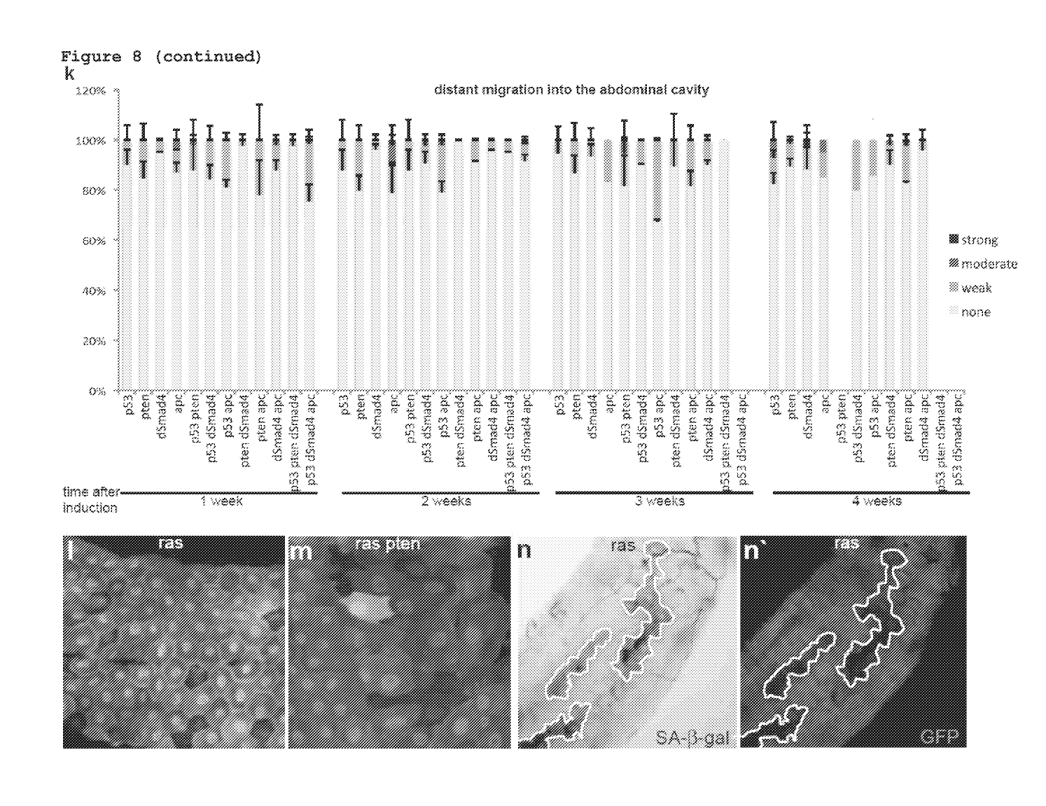

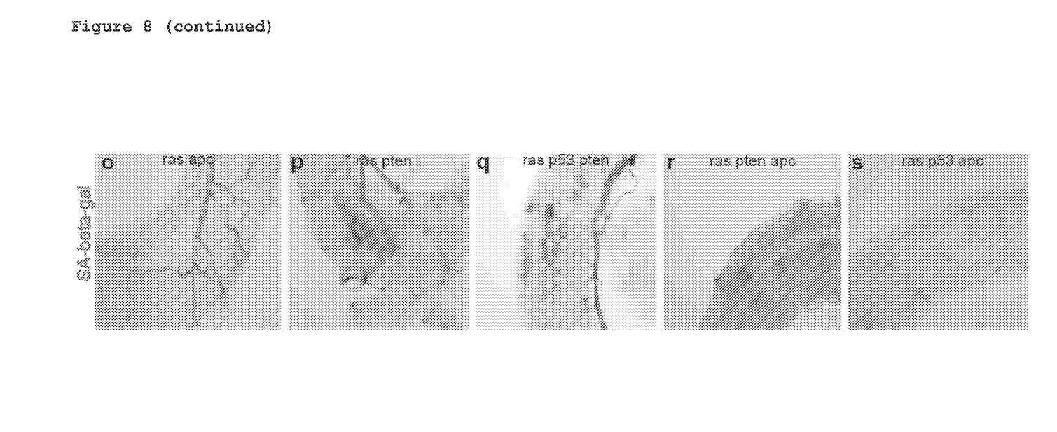

As was recently reported (18), ras^{G12V} alone was able to induce proliferation (FIG. 3d); the remaining transgenes, alone or in combination, had no measurable effect on proliferation in the absence of ras^{G12V} (not shown). While ras^{G12V} pten^{RNAi} and ras^{G12V} apc^{RNAi} exhibited strong proliferation phenotypes comparable to ras^{G12V} alone, the strongest proliferation phenotype was observed with the triple combination ras^{G12V} pten^{RNAi} apc^{RNAi} (FIG. 8). By contrast, p53^{RNAi} consistently inhibited proliferation (FIG. 3e, FIG. 8). A similar reduction of self-renewal capacity of tissues without p53 function has also been reported in mammalian systems and attributed to the accumulation of persistent DNA damage over time (19,20). Overall, these findings indicate that ras^{G12V} is necessary and sufficient to induce proliferation; pten^{RNAi} and apc^{RNAi} strongly synergize with ras^{G12V} while p53^{RNAi} inhibits proliferation.

A similar analysis of the multilayering phenotype was carried out. The absence of multilayering in ras^{G12V} p53^{RNAi} pten^{RNAi} smad4^{RNAi} (FIGS. 3f,g) suggested that apc^{RNAi} could be responsible for this phenotype, though neither apc^{RNAi} nor the other transgenes could induce multilayering by themselves (FIG. 3h,i, not shown). Analysis of double and triple combinations indicated that multilayering of the hindgut epithelium required a combination of ras^{G12V} and apc^{RNAi} (FIG. 3j).

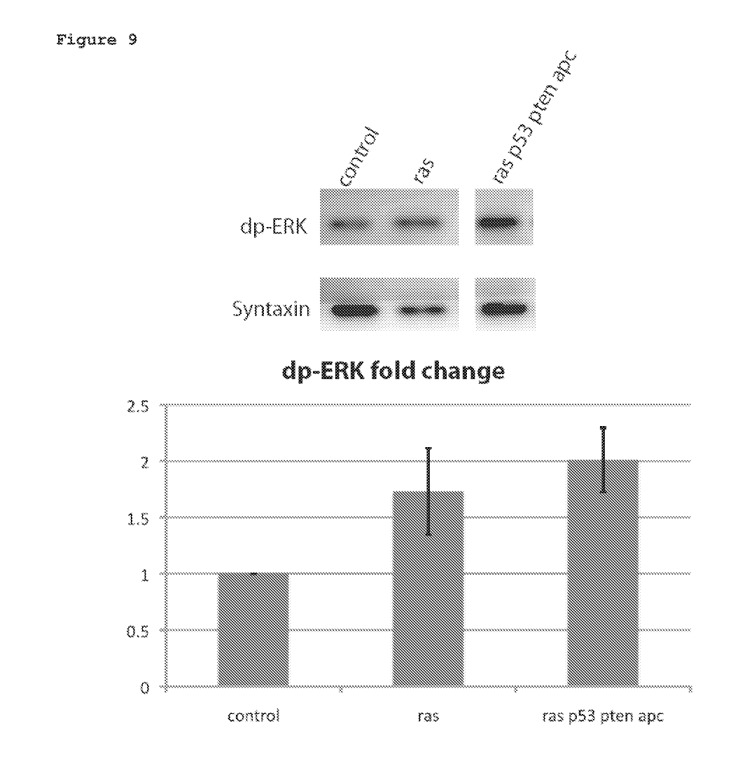

As described above, ras^{G12V} p53^{RNAi} pten^{RNAi} apc^{RNAi} displayed a highly penetrant distant migration phenotype into the abdominal cavity one week after induction of the transgenes (FIGS. 2x, 3k). Removing ras^{G12V} led to a significant suppression of distant migration as p53^{RNAi}, pten^{RNAi}, and apc^{RNAi}-alone or in combination-displayed low levels of migration (FIGS. 3k, 8). Conversely and consistent with previous observations (18), rasc^{G12V} alone had a strong migration phenotype though less penetrant than the quadruple (FIG. 3k); p53^{RNAi}, pten^{RNAi} and apc^{RNAi} each significantly enhanced this migration (FIG. 3k). Despite the strong proliferation and migration phenotypes induced in ras^{G12V} animals, ras^{G12V} overexpression in the hindgut leads to a modest 2-fold increase in MAPK activity (FIG. 9), ruling out the possibility that these phenotypes are caused by non-physiologically high levels of ras^{G12V} expression.

Curiously, pairing each transgene with ras^{G12V} showed stronger migration phenotypes than ras^{G12V} p₅₃^{RNAi} pten^{RNAi} apc^{RNAi} or the various triple combinations (FIG. 3k). As combining 3-4 transgenes together weakened the migration phenotype, the possibility that targeting all four pathways provide other benefits that outweigh reduction in distant migration was explored.

### Example 7

### Advantages Conferred to Tumors by Multigenic Combinations

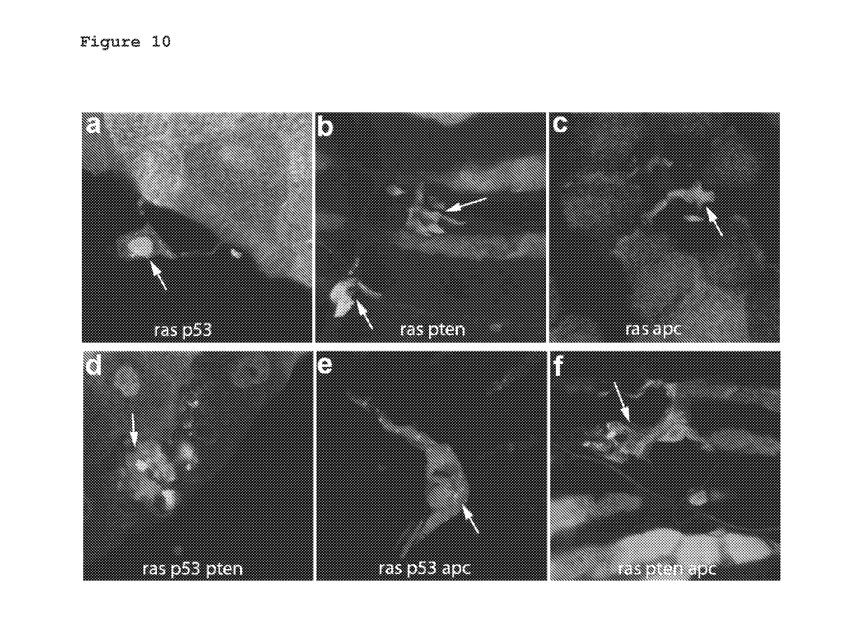

To better understand the role that genetic complexity plays in tumor progression, the transformed cells themselves were more carefully examined. For example, both ras^{G12V} alone and ras^{G12V} p53^{RNAi} pten^{RNAi} apc^{RNAi} hindguts displayed strong distant migration phenotypes (FIG. 3k). However, the migrating cells carrying these combinations were phenotypically distinct. Migrating ras^{G12V} cells were small and extended short processes (FIG. 31). Migrating ras^{G12V} p53^{RNAi} pten^{RNAi} apc^{RNAi} cells were significantly larger with long processes (FIG. 3m) and more extensive contacts with the overlying tracheal branches. Similar large migrating cells were observed in triple combinations but not in doubles (FIG. 10). These data show that that the triple and quadruple combinations led to a more complete transformation process, yielding apparently more robust migrating cells.

Apoptosis is an important cellular defense against cancer, and tumor cells acquire methods to evade it (21). Control tissue displayed no detectable caspase activation as assessed by Dronec activity, a primary initiator caspase (22)(FIG. 3n).

Within hindguts expressing single transgenes, high levels of caspase activity were observed with ras^{G12V} alone; weaker levels were observed with pten^{RNAi} and none with p53^{RNAi} or apc^{RNAi} (FIGS. 3p, 8; not shown). In contrast to ras^{G12V} hindguts, no caspase activation was detected in ras^{G12V} p53^{RNAi} pten^{RNAi} apc^{RNAi} hindguts (FIG. 30), indicating that one role of reducing tumor suppressors is to block ras^{G12V}-dependent caspase cleavage within tumors.

Specific sub-combinations were also examined. Caspase activation was not detected in ras^{G12V} p53^{RNAi} hindguts (FIG. 3r), indicating that intact p53 activity is required to activate the apoptosis pathway. ras^{G12V} pten^{RNAi} hindguts exhibited weak caspase activity (FIG. 8) indicating that reducing pten suppresses apoptosis in the context of ras^{G12V}. ras^{G12V} apc^{RNAi} hindguts showed high levels of caspase activity (FIG. 8). Further complexity emerged within triple combinations: p53^{RNAi} failed to block caspase cleavage in the presence of ras^{G12V} apc^{RNAi} but p53^{RNAi} plus pten^{RNAi} suppressed caspase cleavage induced by ras^{G12V} alone or ras^{G12V} apc^{RNAi} (FIG. 3w, FIG. 8). These data show that, in the transformation process, higher pathway complexity favors a block in apoptosis.

Oncogenic stress-induced by Ras or Raf activation or by loss of Pten-can lead to oncogene-induced senescence, another important cellular defense against malignant progression (23,24). It was found herein that subset of hindgut cells lost GFP expression, most notably those bearing ras^{G12V} or ras^{G12V} pten^{RNAi} or, to a lesser extent, pten^{RNAi} alone (FIG. 8; not shown). These same GFP-negative cells were positive for the senescence marker SA-β-gal (senescence-associated-β-gal; e.g., FIGS. 3u, 8), indicating emergence of at least some aspects of cellular senescence.

By contrast, SA-β-gal-positive cells were rarely observed in ras^{G12V} p53^{RNAi} pten^{RNAi} apc^{RNAi}, ras^{G12V} p53^{RNAi}, ras^{G12V} apc^{RNAi}, or ras^{G12V} p53^{RNAi} apc^{RNAi} hindguts (FIGS. 3t, 3v, 8), indicating that p53^{RNAi} and apc^{RNAi} oppose SA-β-gal activation. Numerous SA-β-gal positive cells were still observed in ras^{G12V} p53^{RNAi} pten^{RNAi} and a few in ras^{G12V} pten^{RNAi} apc^{RNAi} (FIG. 8), indicating that reducing p53 plus apc is required to prevent loss of SA-β-gal in the context of ras^{G12V} pten^{RNAi}. These results again indicate the emergent properties by which the four loci interact to promote tumorigenesis, in this case by blocking a senescence-like phenotype. The phenotypes observed in the quadruple combinations and the interactions observed between the four transgenes leading to each of these phenotypes are summarized in FIGS. 3w,x.

### Example 8

### Quadruple-Hit Tumors are Resistant to Targeted Therapeutic Agents

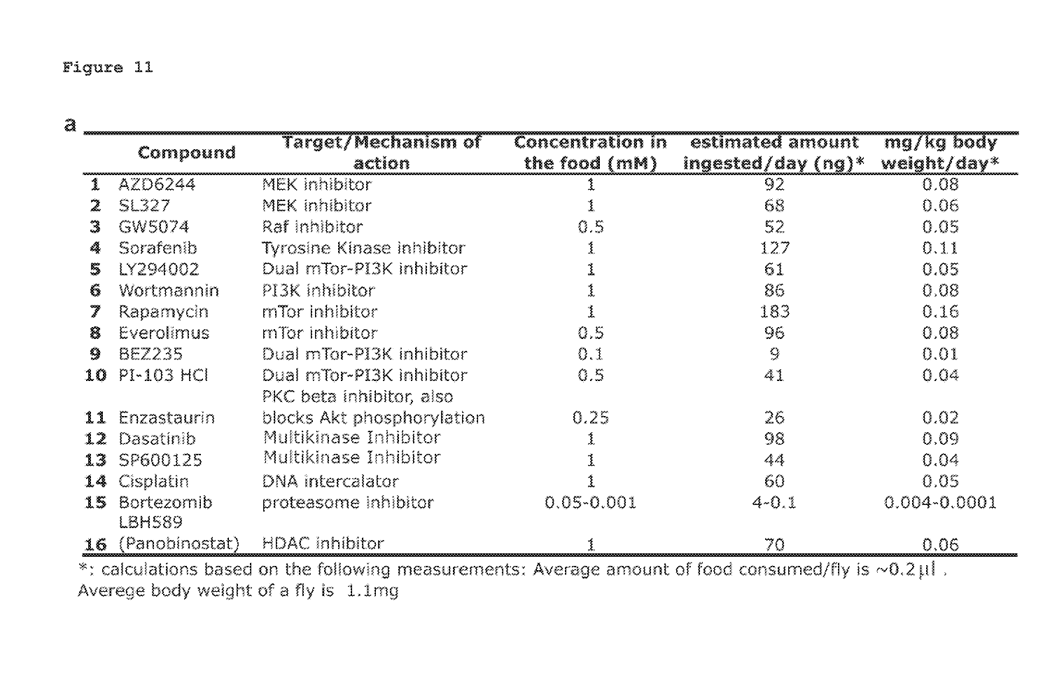

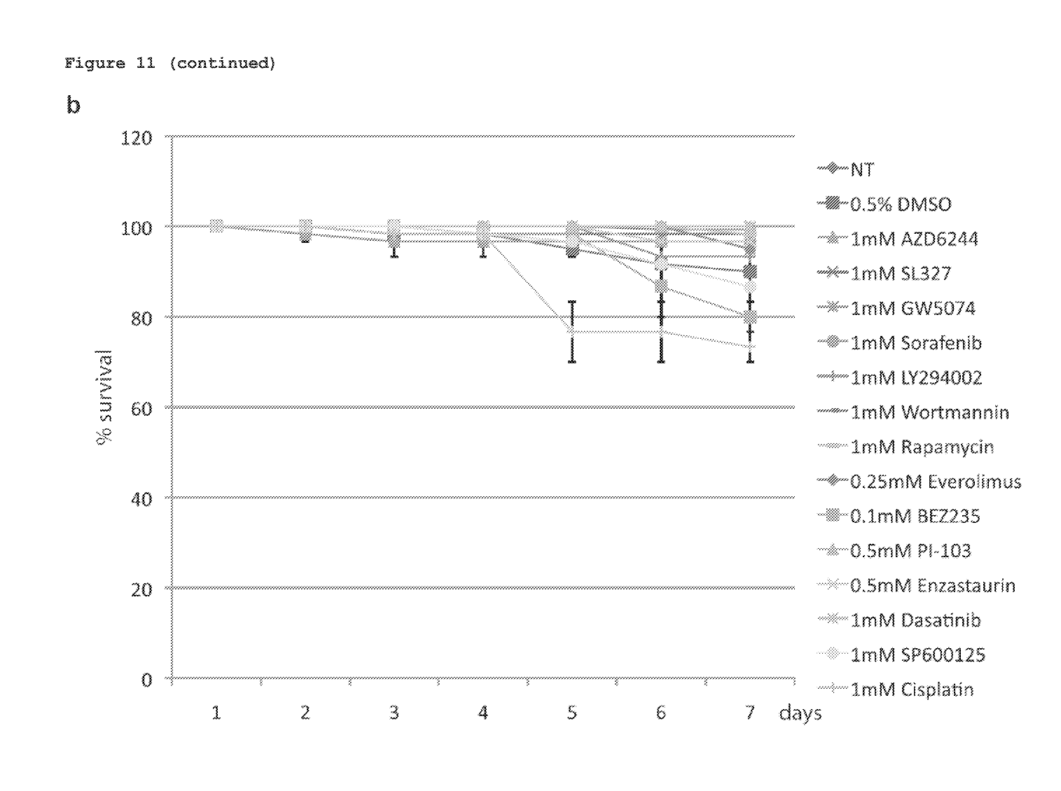

Given the low success rate of drug approvals for colorectal cancer (3) the study herein asked whether differences in genetic complexity influence a tumor model's response to drugs. Sixteen compounds that target key cancer relevant pathways and cellular processes were selected (FIG. 11); many of these compounds are currently in clinical trials including for colorectal cancer (25-27). Commencing the day of transgene induction, animals were fed compounds for one week and then scored for issemination into the abdominal cavity. Final compound concentrations in the media ranged from 1 μM-1 mM, corresponding to approximately 10-200 ng/day/animal based on adult feeding rates (FIG. 11).

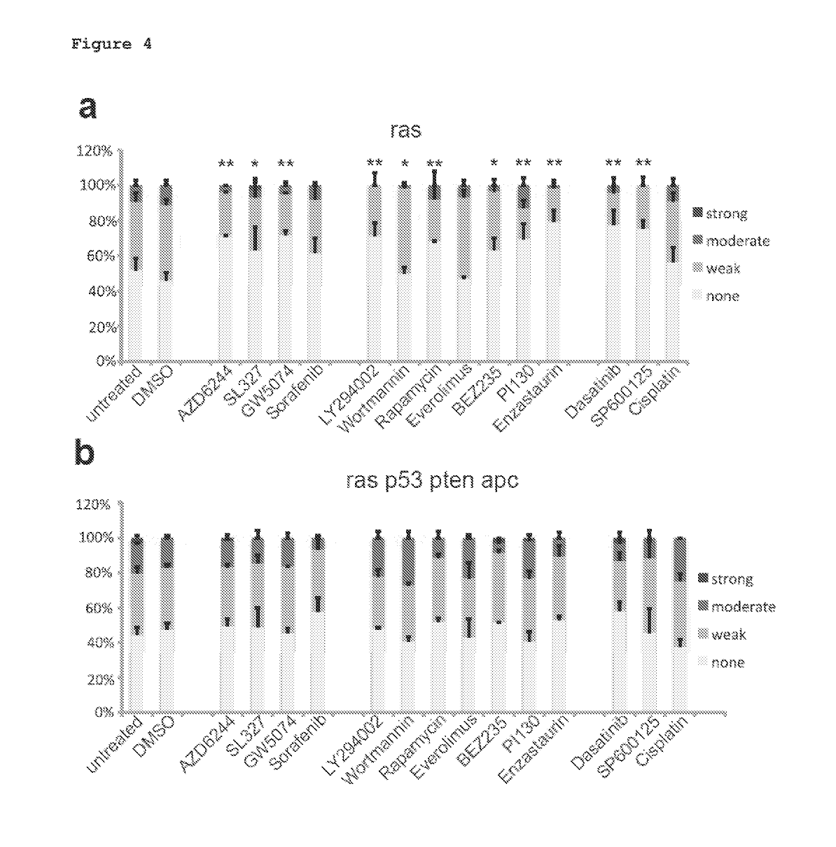

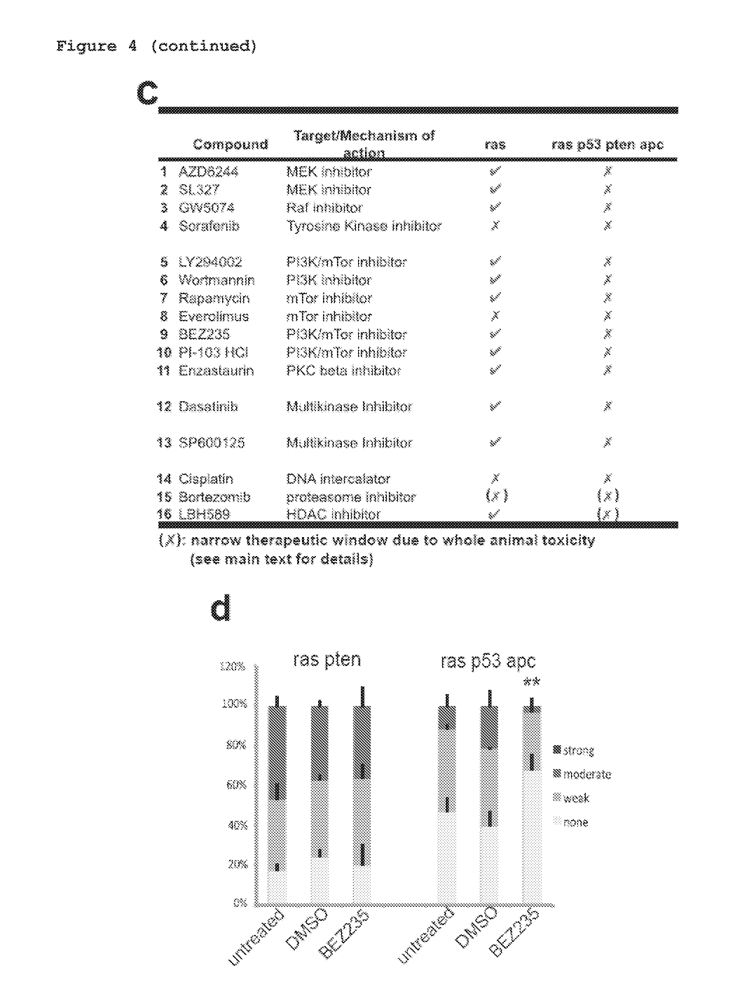

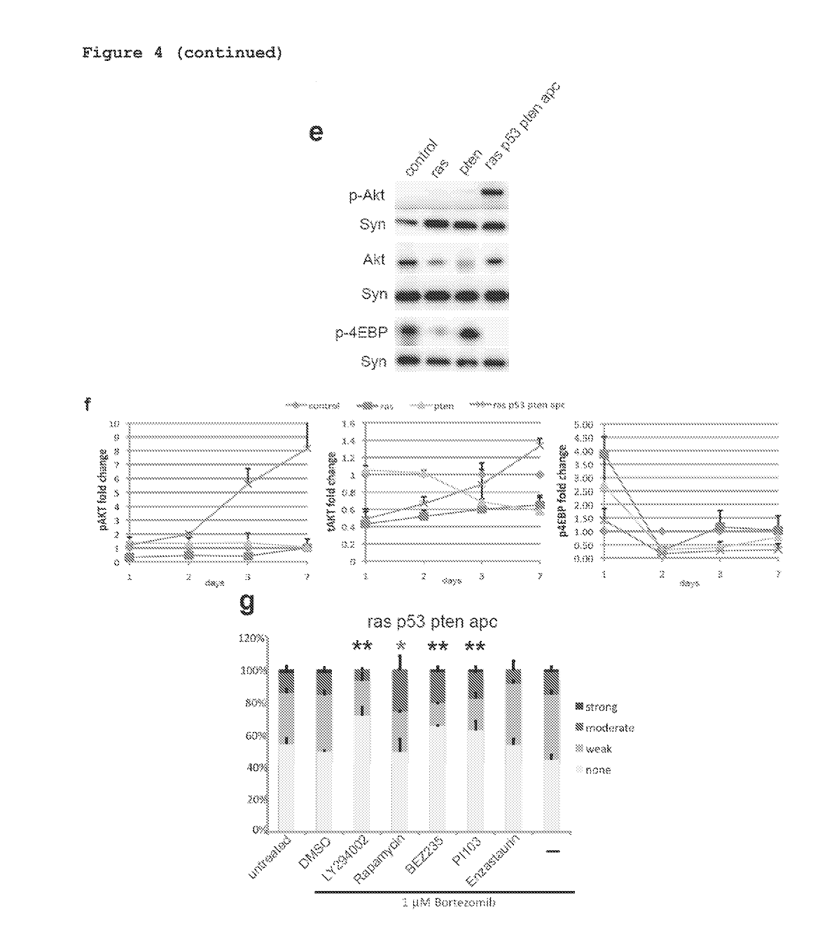

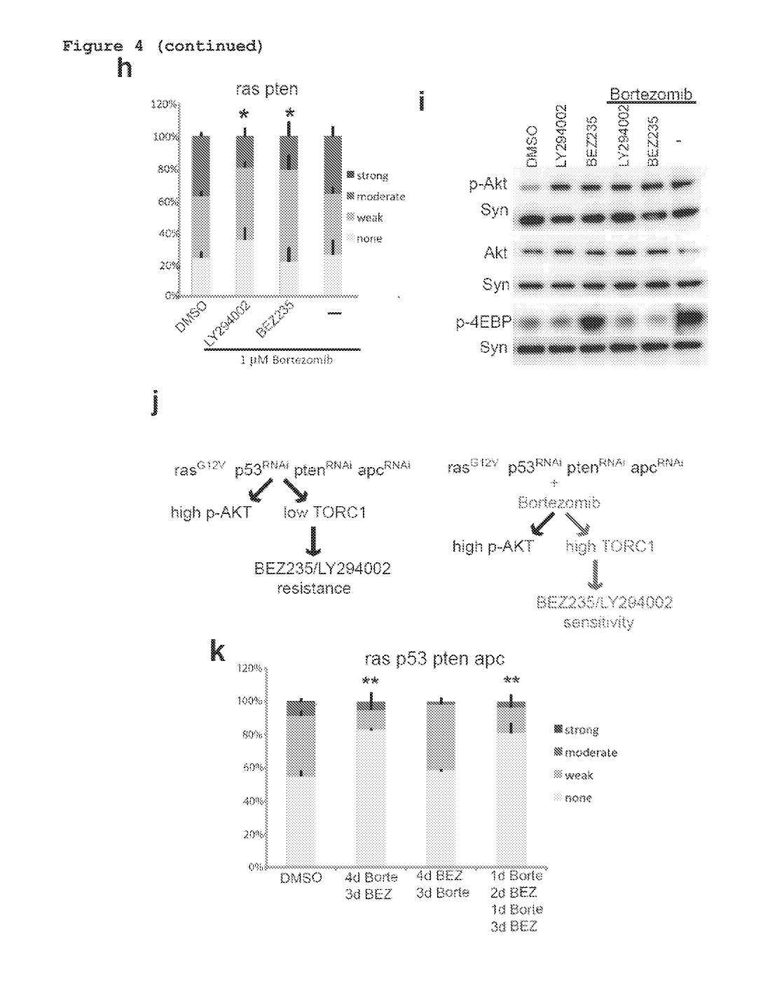

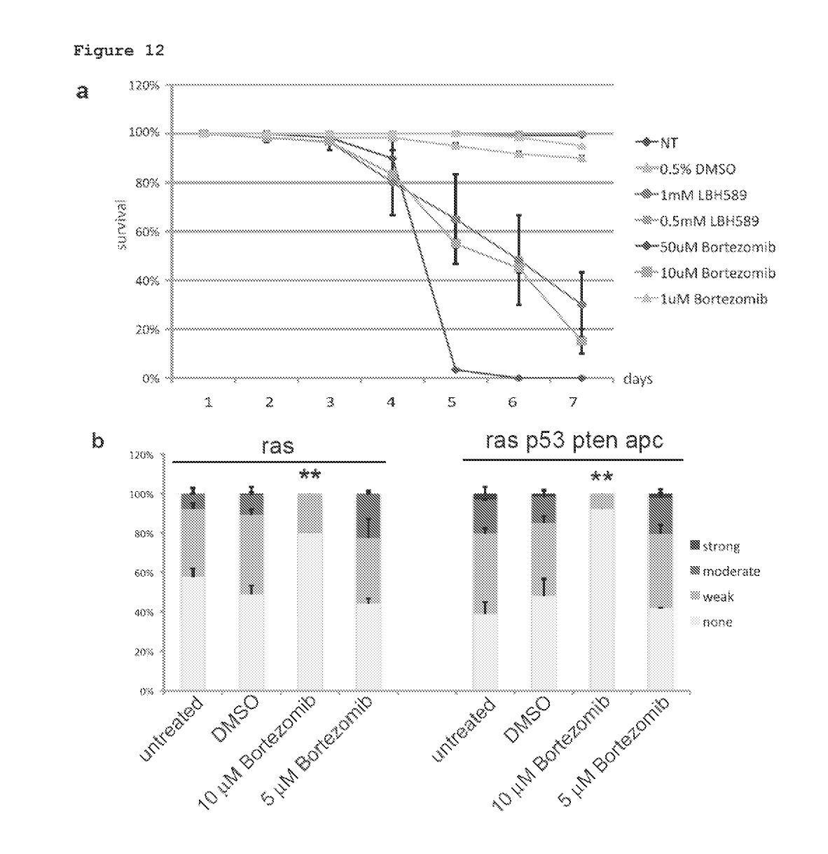

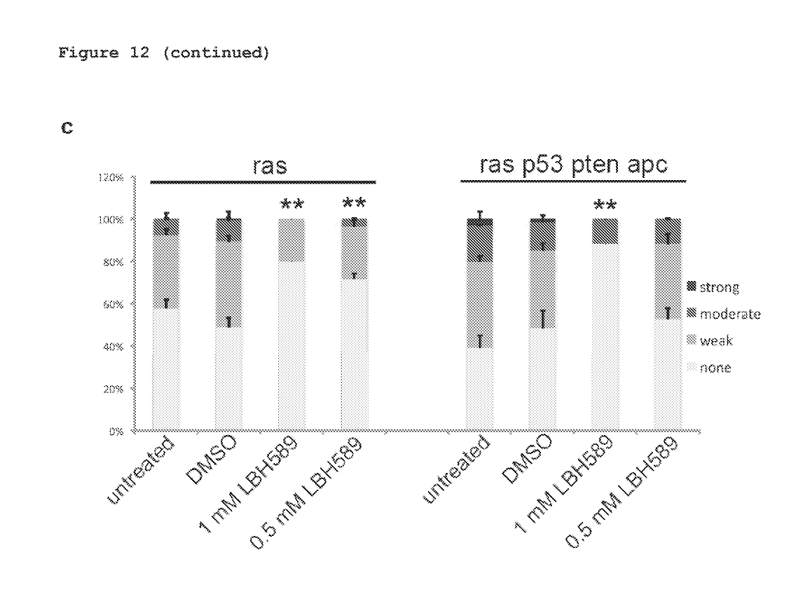

In ras^{G12V} animals, distant migration was significantly suppressed by 12/16 compounds tested, including inhibitors of Raf, MEK, PI3K, mTor, Src, and JNK (FIG. 4a,c). Importantly, the four-hit model ras^{G12V} p53^{RNAi} pten^{RNAi} apc^{RNAi} exhibited a different response: none of these compounds had a statistically significant effect on distant migration (FIGS. 4b,c). None of the compounds showed significant whole animal toxicity at effective doses (FIG. 11) except for the proteosome inhibitor bortezomib (velcade), which only showed efficacy at doses that otherwise killed more than 70% of the animals (FIGS. S7, 4c). The HDAC inhibitor LBH589 (panobinostat) was effective against ras^{G12V} but not against ras^{G12V} p53^{RNAi} pten^{RNAi} apc^{RNAi} at non-toxic doses (FIGS. 4c, 12).

Together, the data herein indicate that drug resistance is another emergent property of multigenic combinations: activating multiple cancer pathways can lead to resistance to a wide range of anti-cancer agents.

### Example 9

### Drug Response as an Emergent Property of Multigenic Combinations

BEZ235 was focused on to determine the transgene(s) responsible for the emergent drug resistance observed in ras^{G12V} p53^{RNAi} pten^{RNAi} apc^{RNAi} animals. It is the first PI3K/mTor inhibitor to enter clinical trials; a phase I trial targeting a spectrum of solid tumors including colorectal is ongoing (25,28). Removing pten^{RNAi} from the quadruple combination (ras^{G12V} p53^{RNAi} apc^{RNAi}) rendered the animals sensitive to BEZ235 (FIG. 4d). Conversely, adding ptensA to ras^{G12V} (ras^{G12V} pten^{RNAi}) was sufficient to direct resistance to BEZ235 (FIG. 4d). This study shows that resistance to the PI3K/mTor inhibitor BEZ235 is an emergent property of the ras^{G12V} pten^{RNAi} combination; the results herein suggest that patients with these two pathways activated may respond more poorly to PI3K pathway inhibitors.

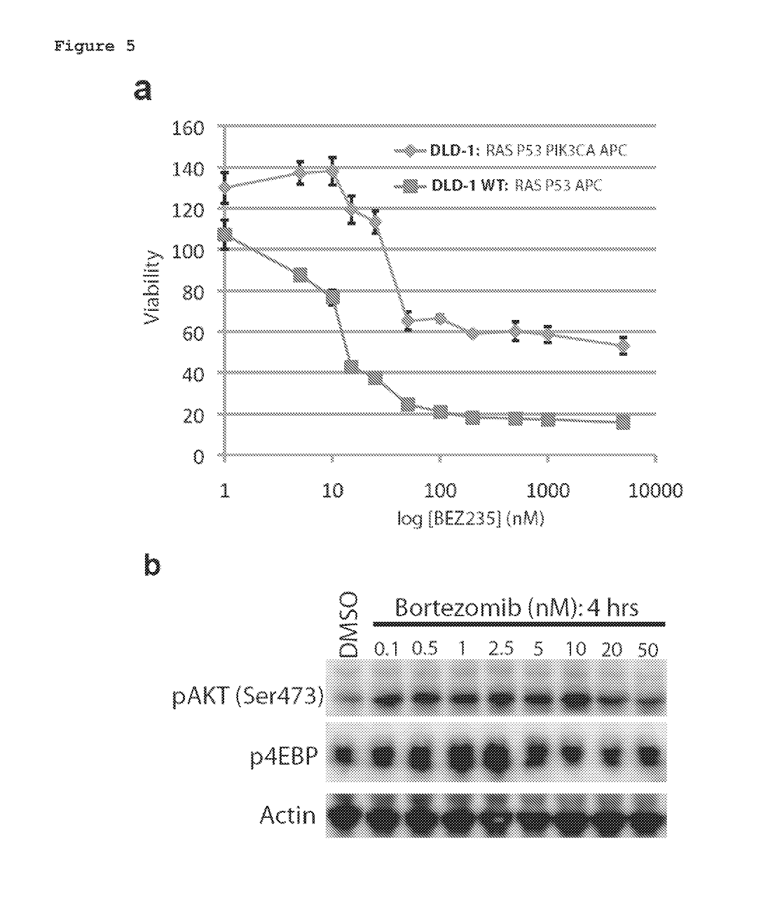

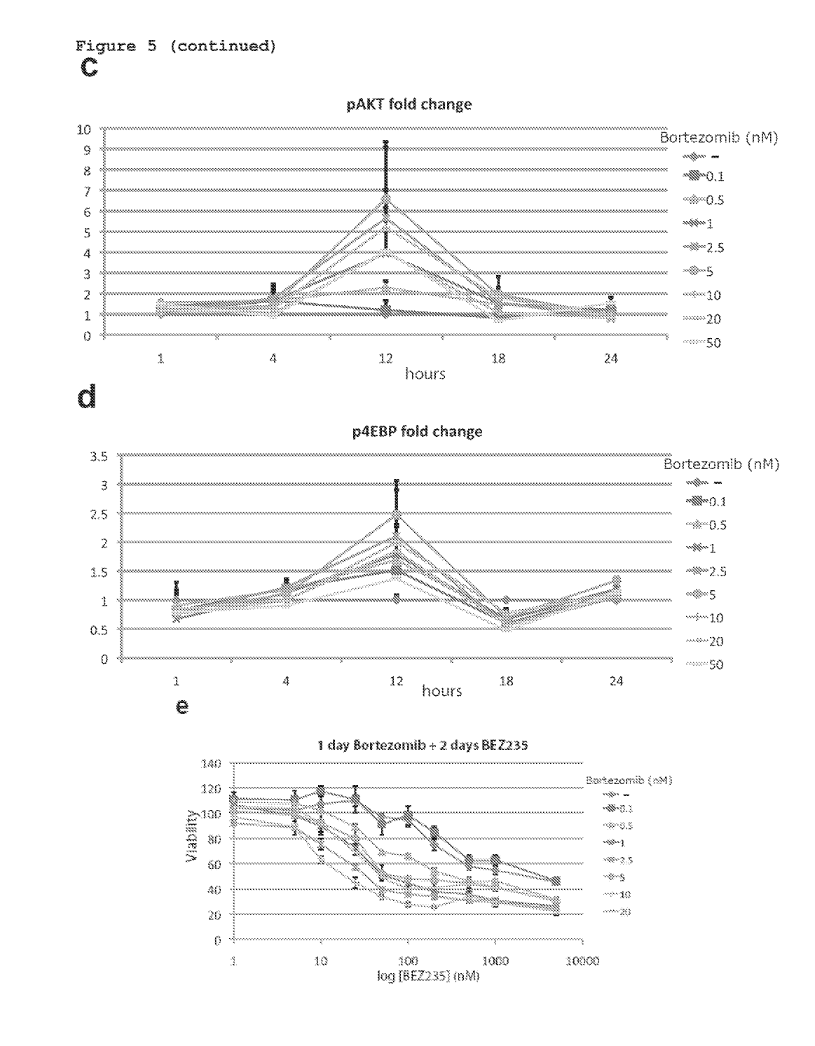

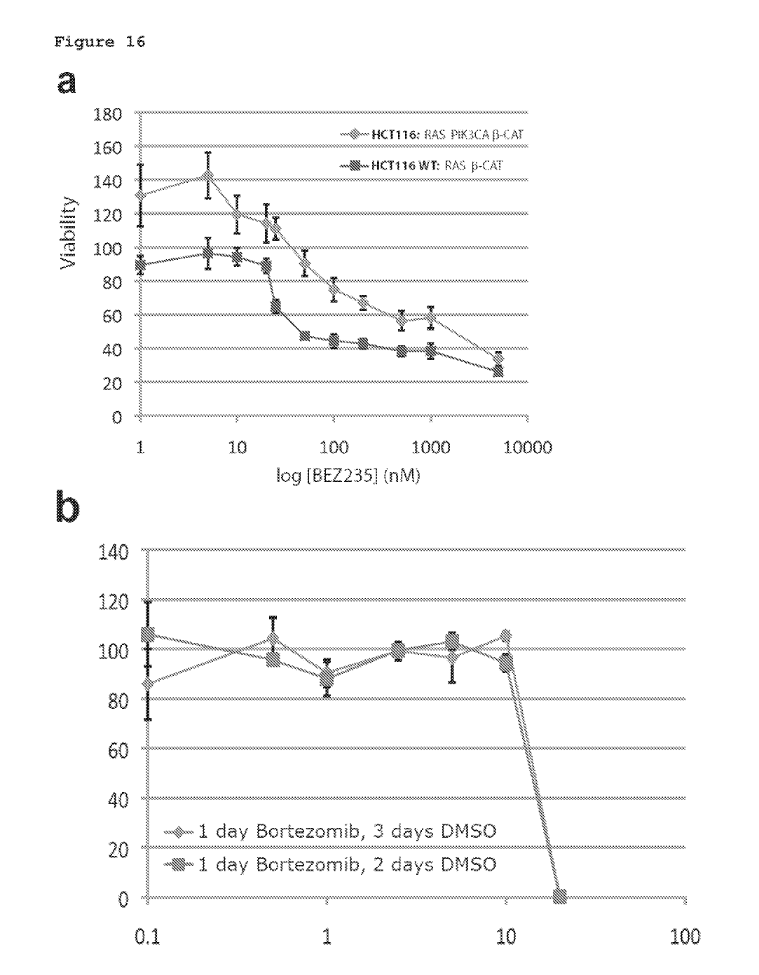

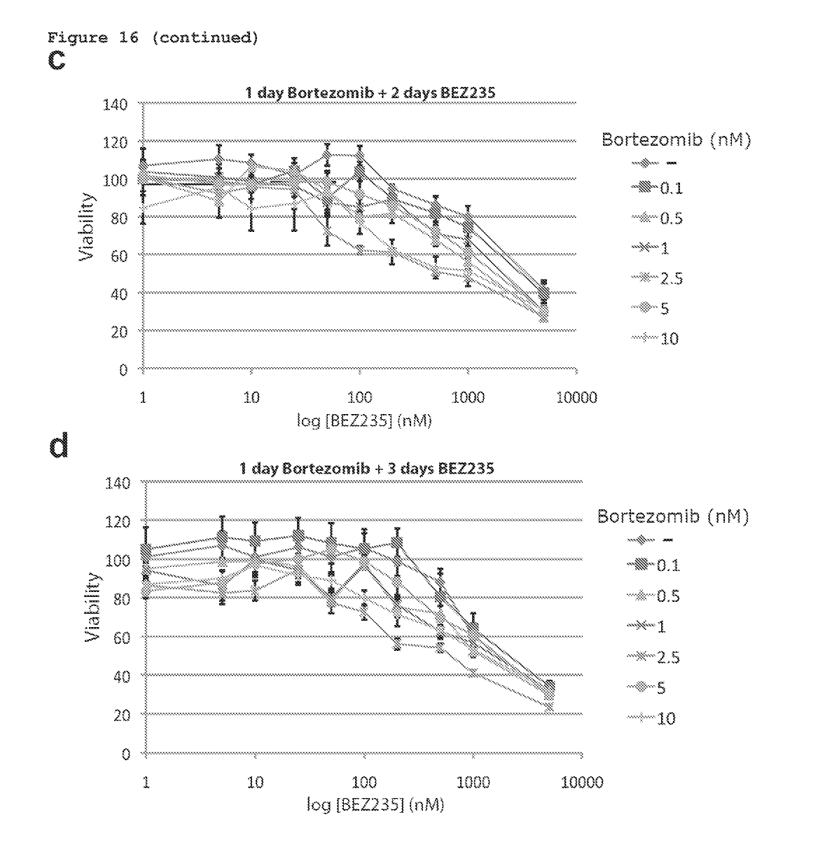

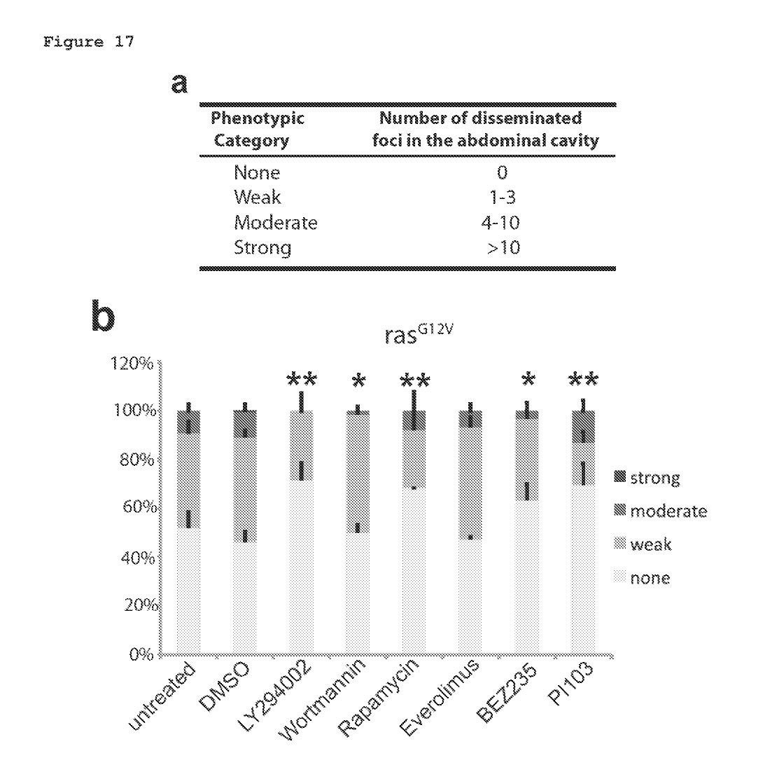

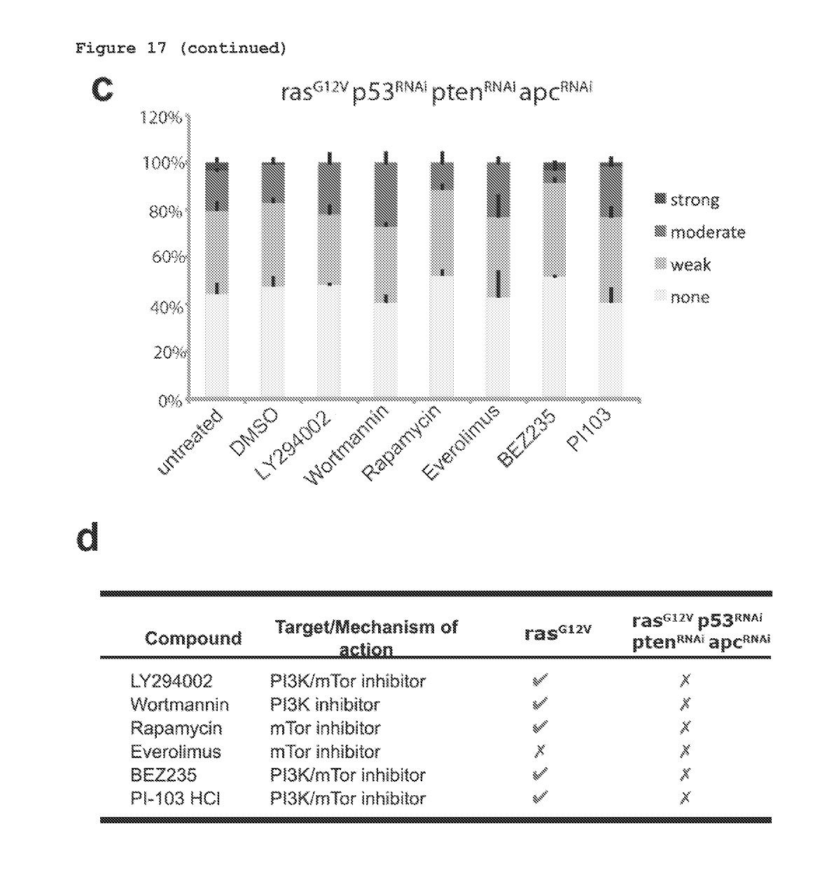

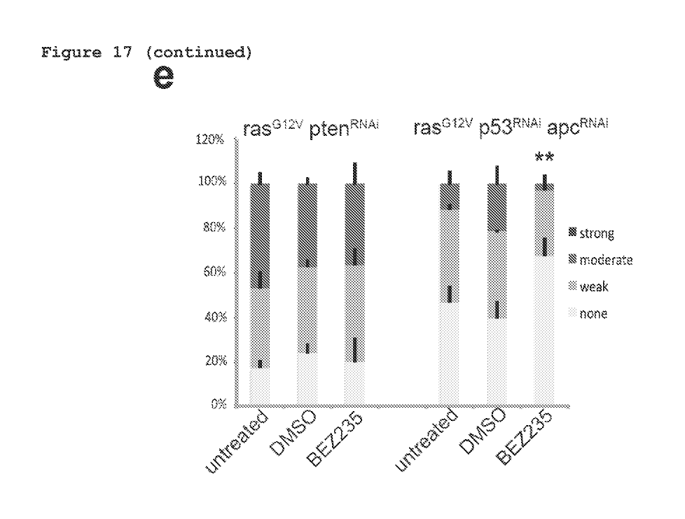

Whether the predictions herein regarding BEZ235 resistance based on pathway activation status were also true in human colon cancer cell lines was tested. It was found that DLD-1 cells contain mutations in Ras, p53, APC, and the PI3K pathway component PIK3CA, similar to the four-hit model herein. This human colorectal cancer cell line was more resistant to BEZ235 than the derivative line DLD-1-WT, which is restored for normal PIK3CA function (FIG. 5a). Furthermore, another colorectal cancer cell line that shows coactivation of Ras/MAPK and PI3K pathways, HCT116 (Ras PIK3CA CTNN1) was also more resistant to BEZ235 than its PI3K pathway wildtype counterpart, HCT116-WT (FIG. 16a).

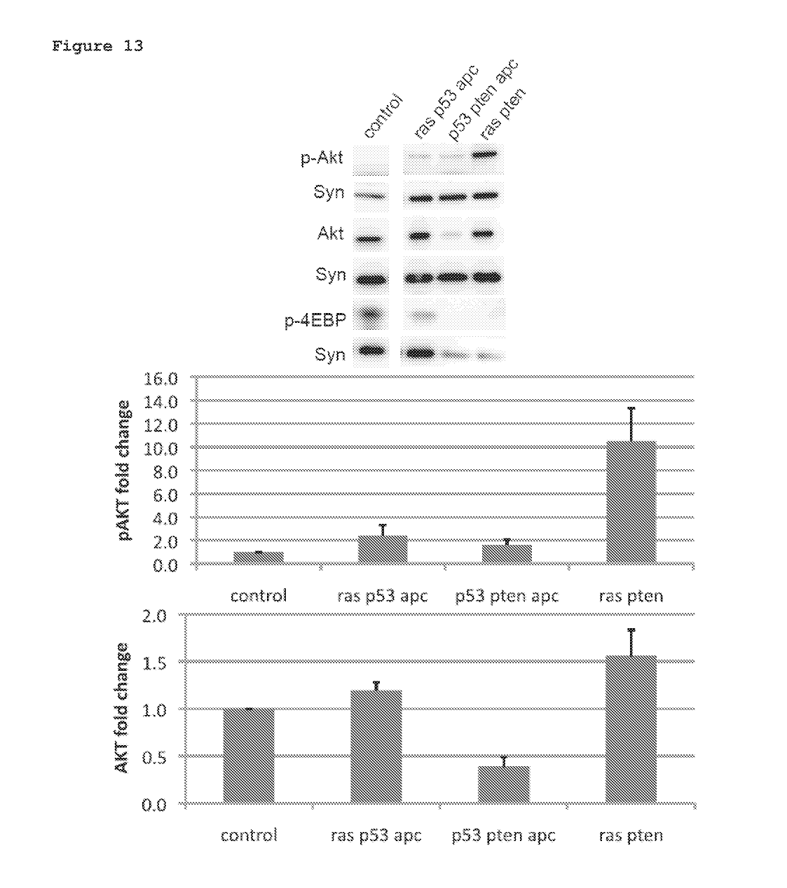

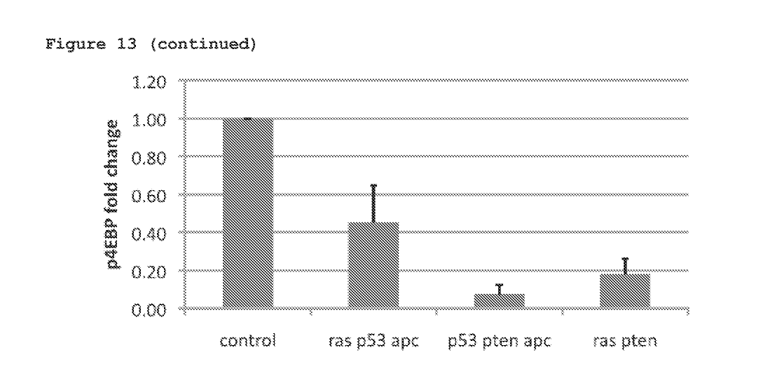

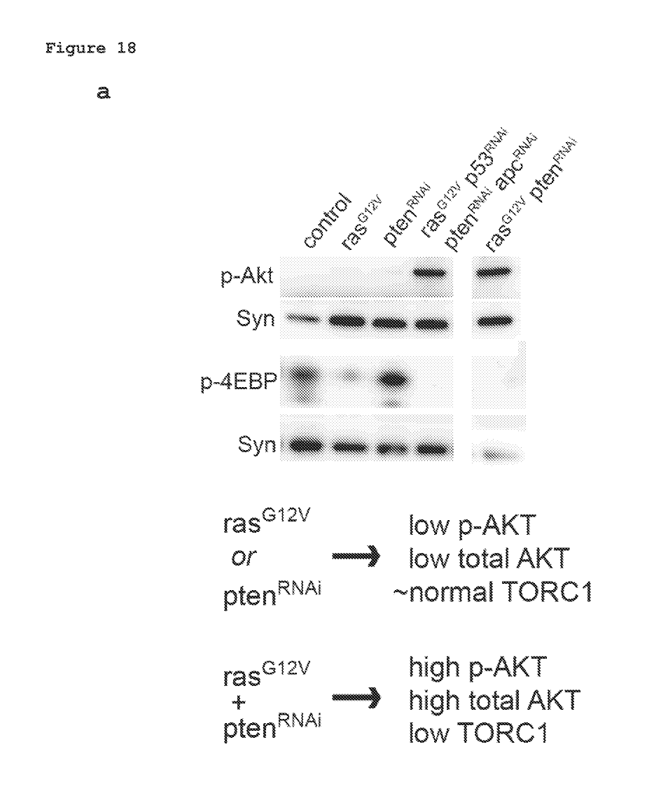

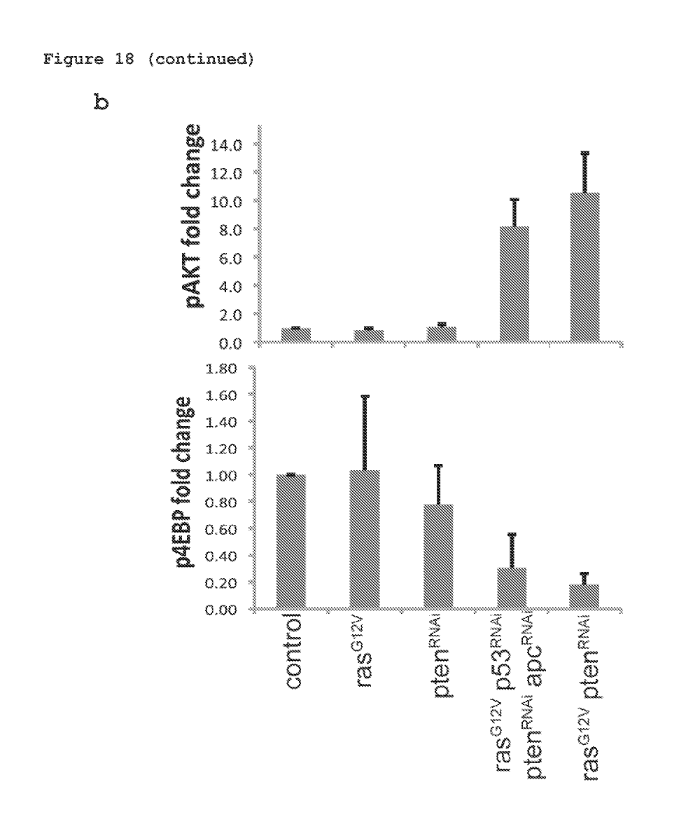

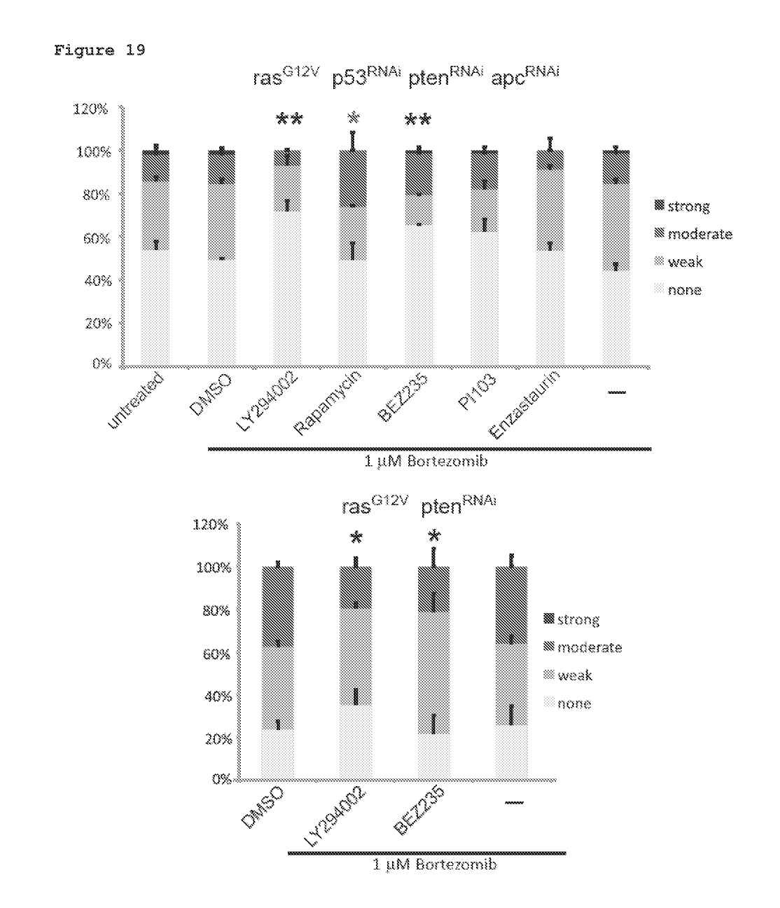

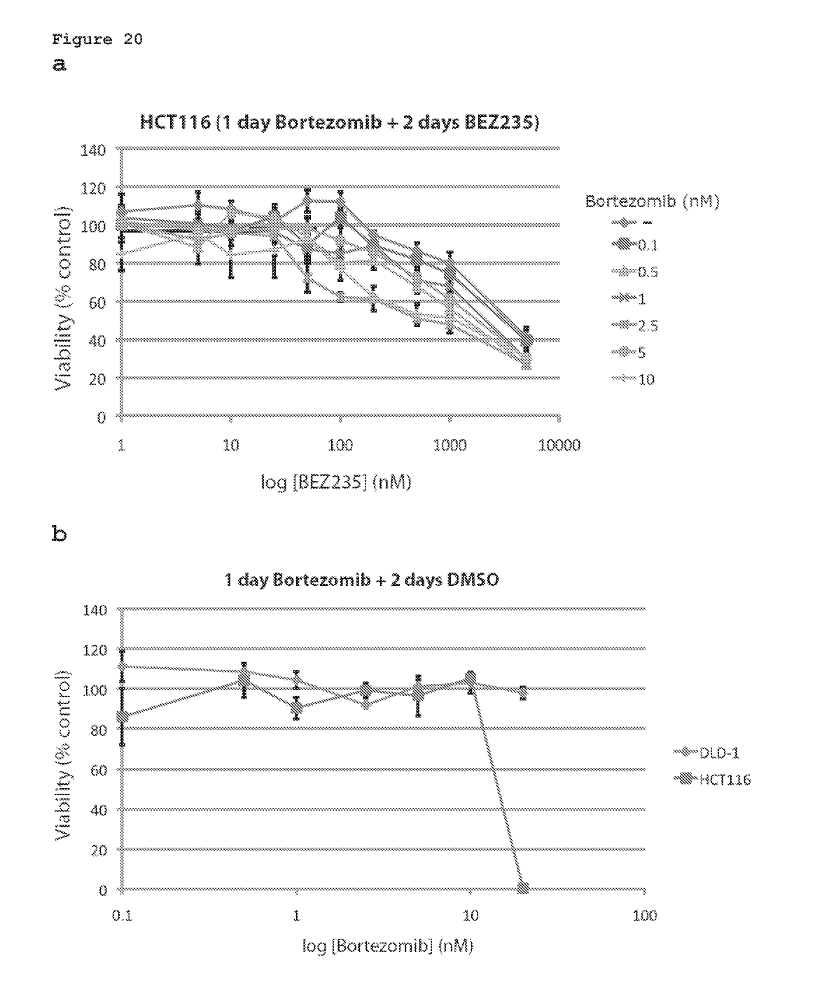

The transgenic animals provided the opportunity to explore the in situ dynamics of PI3K pathway activation in the context of a transformed gut and in the presence of candidate therapeutics. PI3K activation leads to phosphorylation of AKT (p-AKT), which in turn has multiple targets that regulate key aspects of cell survival, growth and metabolism including Tor Complex 1 (TORC1) (29,30). Seven days after transgene induction, ras^{G12V} p53^{RNAi} pten^{RNAi} apc^{RNAi} cells reached a steady state with (i) high levels of p-AKT and (ii) very low levels of TORC1 activity over time (as indicated by 4EBP phosphorylation) (FIG. 4e-f, FIG. 13), a pattern that was recapitulated ras^{G12V} pten^{RNAi} animals (FIG. 18a,b). Time course analysis of pathway activation in ras^{G12V} p53^{RNAi} pten^{RNAi} apc^{RNAi} hindguts showed a continuous increase in p-AKT levels and a concomitant decrease in TORC1 activity (as indicated by 4EBP phosphorylation; FIG. 4f), indicating that while both AKT and TORC1 were activated early on, TORC1 activity decreased over time, while pAKT levels kept rising. By contrast, while single transgene ras^{G12V} or pten^{RNAi} animals began with a similar activity profile, by day seven (i) p-AKT levels were very low and (ii) p-4EBP levels rose back to normal levels (ras^{G12V} reduced ART more rapidly than ptenRNAi (FIG. 4f).

Interestingly, this cellular state of high pAKT and low TORC1 activity can also be induced by FoxO to maintain energy homeostasis in response to physiological stress both in mammalian cells and in Drosophila (31,32). It is possible that this steady state of PI3K pathway activity could provide an advantage to tumor cells whose energy metabolism is altered as a result of oncogenic transformation.

By contrast, while single transgene ras^{G12V} or pten^{RNAi} animals began with a similar activity profile, by day seven (i) p-AKT and total AKT levels were very low and (ii) p-4EBP levels rose back to normal levels (ras^{G12V} reduced AKT more rapidly than pten^{RNAi}; FIG. 4e-f). This altered profile likely reflects a previously described feedback inhibitory loop that controls AKT protein stability in response to chronic PI3K pathway activation (33); the studies herein find that this feedback loop is disrupted when ras^{G12V} and pten^{RNAi} are present together. That is, the key to drug sensitivity is high (normal) TORC1 activity, with p-4EBP levels serving as a biomarker.

### Example 10

### Low Dose Bortezomib Treatment Overcomes Resistance to PI3K Pathway Inhibitors

Signaling pathways rely on constant protein turnover to regulate their activity. It was reasoned herein that inhibiting protein degradation might alter the steady state of PI3K pathway activity we observed in our model and help overcome drug resistance in ras^{G12V} p53^{RNAi} pten^{RNAi} apc^{RNAi} animals. Bortezomib has been reported to both inhibit and activate the PI3K pathway depending on the cell type, dose and duration of treatment (34-36). In exploring bortezomib's effects on signaling in our model, it was noted herein that low doses directed upregulation of PI3K and TORC1 activity (FIG. 4i), a state the experiments herein associated with drug sensitivity. It was hypothesized that this increase in TORC1 activity would render ras^{G12V} p53^{RNAi} pten^{RNAi} apc^{RNAi} hindguts sensitive to PI3K/mTor inhibitors. In fact, low non-toxic concentrations (1 μM, i.e. −10 ng/day/animal) of bortezomib synergized with the PI3K/mTor inhibitors BEZ235, P1103 and LY294002 to inhibit dissemination within the four-hit model (FIG. 4g). Dissemination induced by the ras^{G12V} pten^{RNAi} double was similarly suppressed by the combination therapy (FIGS. 4h 19a). This indicates that sensitivity to combinatorial therapy that includes bortezomib is an emergent property of ras^{G12V} plus pten^{RNAi}. Interestingly and by contrast, the mTor inhibitor rapamycin enhanced distant migration in combination with bortezomib (FIG. 4g 19c), likely reflecting a previously described feedback activation of the PI3K pathway on Ras signaling (37) and suggesting this combination would not be useful as a therapeutic.

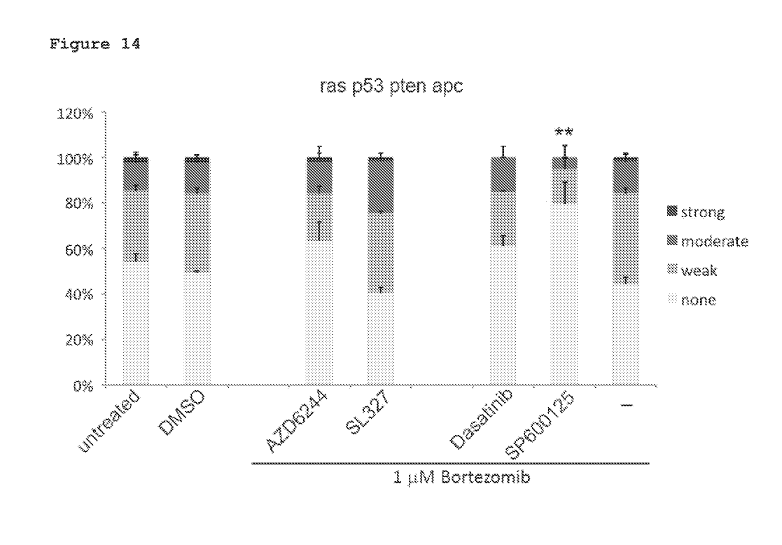

The concept of activating TORC1 activity to promote drug sensitivity was further supported by examining PI3K signaling after combinatorial therapy (FIG. 4i). Bortezomib treatment alone led to an increase in p-4EBP levels, a condition that confers drug sensitivity (FIG. 4i,j). Indeed, combining bortezomib with either BEZ235 or LY294002 reduced p-4EBP back to baseline levels (FIG. 4i,j); this reduction of TORC1 activity was accompanied by efficacy against the quadruple combination (FIG. 4g). If re-activating TORC1 by bortezomib drives dependence on the pathway, thereby rendering these cells sensitive to PI3K pathway inhibition, sequential treatment of animals with bortezomib followed by BEZ235 should also be effective, but not when the order of the treatment is reversed. This is precisely what we observed (FIGS. 4k 19c). Indeed, sequential therapy was much more effective than concurrent feeding of bortezomib andBEZ235 (19c). These data are consistent with the view that TORC1 activity is a key biomarker and determinant of a tumor's sensitivity to at least two PI3K pathway inhibitors. Bortezomib's activity in combination with other drugs is presented in FIG. 14.

### Example 11

### Validation in Human Colorectal Cancer Cell Lines

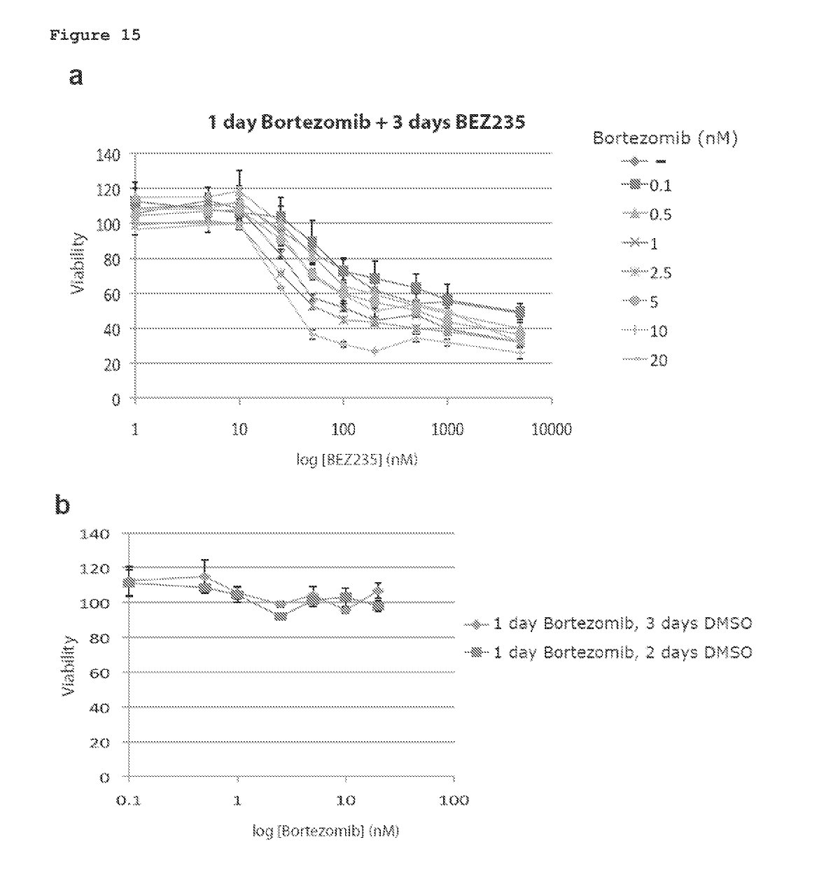

Whether the predictions herein regarding BEZ235 resistance based on pathway activation status held true in human colon cancer cell lines was tested next. The human colorectal cancer cell line DLD-1 contains mutations in Ras, p53, APC, and the PI3K pathway component PIK3CA, a mutational status similar to our four-hit Drosophila model. DLD-1 proved more resistant to BEZ235 than the derivative line DLD-1-WT, in which normal PIK3CA function is restored (38) (FIG. 5a). Also similar to our observations in flies, bortezomib activated the PI3K pathway in these cell lines at doses and durations that did not affect survival (FIG. 5b-d, FIG. 15). Importantly, doses of bortezomib that activated the PI3K pathway also rendered DLD-1 cells sensitive to BEZ235 treatment (FIG. 5e, FIG. 15), mirroring our results in flies. These observations were confirmed with HCT116 cells-another colorectal cancer cell line with co-activation of Ras/MAPK and PI3K pathways-when compared to its PI3K pathway wild type derivative HCT116-WT (38) (FIG. 16).

### Example 12

### DLD-1 Xenograft Study

GOAL: To determine efficacy of Bortezomib-BEZ235 combination in DLD1 xenograft model.

### Experiment Layout:

Mice: Athymic females
Cells: DLD1 cells 10 million cells s.c. single flank with matrigel
Media: DMEM, 10% FBS+P/S
Drugs: Bortezomib i.p.
   BEZ235 p.o.
Vehicle: saline (bortezomib)
   10 animals/group. Start treatments when tumors are ˜80-100 mm³ and continue for 3 weeks, Monitor tumor growth twice a week:
      1.Vehicle p.o. QD Mon-Tue-Wed-Thu-Fri
      2.BEZ235 40 mg/kg p.o. Mon-Tue-Wed-Thu-Fri
      3.Bortezomib ip 0.25 mg/kg ip Mon+BEZ235 40 mg/kg p.o. Tue-Wed-Thu-Fri
      4.Bortezomib ip 0.1 mg/kg ip Mon+BEZ235 40 mg/kg p.o. Tue-Wed-Thu-Fri
      5.Bortezomib ip 0.01 mg/kg ip Mon+BEZ235 40 mg/kg p.o. Tue-Wed-Thu-Fri
      6.Bortezomib ip 0.25 mg/kg ip Mon
      7.Bortezomib ip 0.1 mg/kg ip Mon
      8.Bortezomib ip 0.01 mg/kg ip Mon

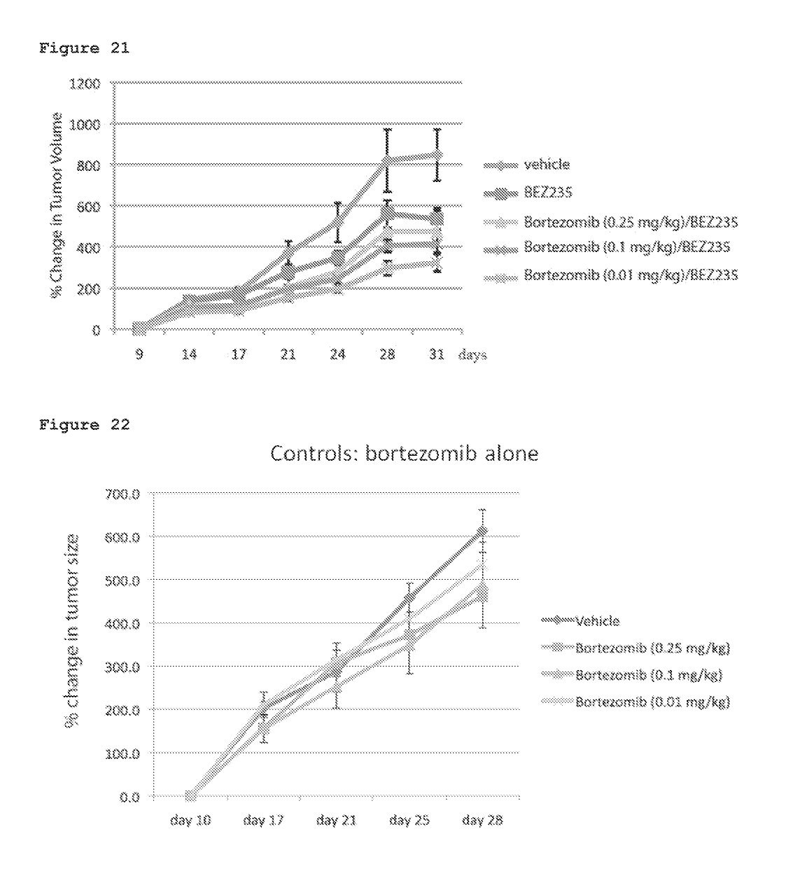

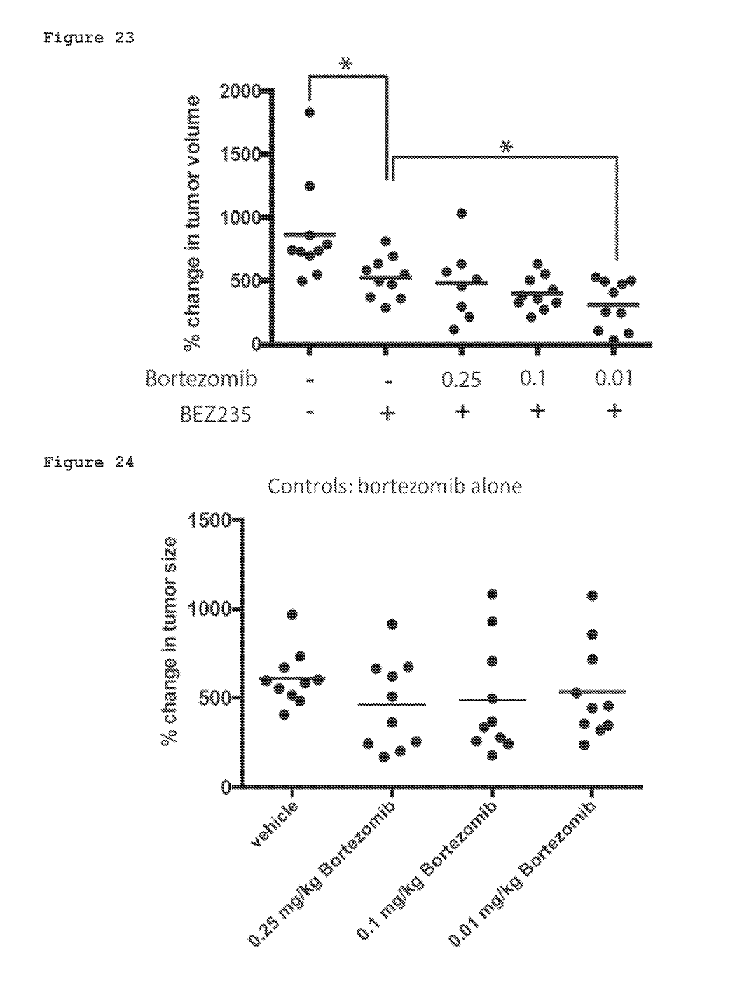

CONCLUSION: The Bortezomib-BEZ235 combination is effective for reducing tumor growth in the DLD1 xenograft model (FIGS. 21-24).

### Discussion

The multigenic and heterogeneous nature of human tumors presents a fundamental challenge for cancer research. To capture this genetic complexity multigenic models of colon cancer were generated in Drosophila-double, triple and quadruple combinations of transgenes representing mutations clustered together in human colon tumors- and targeted them to the adult hindgut. These models recapitulated key features of human cancer, many of which arose as emergent properties of multigenic combinations. Further, using the quadruple combination ras^{G12V} p53^{RNAi} pten^{RNAi} apc^{RNAi} we show that multigenic models exhibit emergent resistance to a panel of drugs; the disclosures herein identify a mechanism of resistance for BEZ235. Combinatorial therapy proved more effective against these multigenic combinations. The disclosures herein demonstrate a novel two-step process of emergent pathway dependence and suppression in both Drosophila and cultured human tumor cells termed ‘induced dependence’. Developing multigenic animal models by referencing cancer genomes is an essential step towards furthering our understanding of a cancer's biology and towards developing effective targeted therapies.

Colon cancer is the second leading cause of cancer-related death in the western world. Its high mortality rate is largely due to the resistance of late stage tumors to targeted therapies. Several highly conserved genes and pathways implicated in colon cancer have been extensively studied in cell culture and mouse models (5,6), but effective therapeutics remain an unmet need. Development of effective targeted therapies will require a better understanding of how genomic changes interact during malignant progression. To this end the subject invention provides a large number of multigenic combinations that target the Drosophila hindgut. These models were designed to reflect sequencing profiles of individual human colon tumors (7-9), taking advantage of extensive conservation of the signaling pathways that direct colon tumors (10). The emergent properties of progressively more complex models were then explored.

### Emergent Properties in Complex Tumors

The disclosures herein demonstrate that targeting the quadruple combination ras^{G12V} p53^{RNAi} pten^{RNAi} apc^{RNAi} to the adult hindgut epithelium led to overproliferation, multilayering, evasion of apoptosis and senescence-like phenotypes, and migration. These are hallmarks of human cancer that reflect the complex and dynamic interactions between individual transgenes within (human tumor-relevant) multigenic combinations (FIGS. 3x,w). Some phenotypes only arose in the presence of multiple transgenes: for instance, multilayering of the hindgut epithelium was an emergent property of ras^{G12V} plus apc^{RNAi}. Also, the behavior of a transgene can change depending on the presence of other transgenes. For instance, while pten^{RNAi} induced caspase activation on its own, it suppressed it in the presence of ras^{G12V}. Finally, some transgenes had complex roles in tumor progression including p53^{RNAi} which was required to evade activation of apoptosis and SA-β-gal but which also reduced tumor cell proliferation.

The findings herein also demonstrate that complex tumors can show resistance to compounds designed for high target specificity. Combinatorial therapy or use of less selective compounds may prove more effective against these tumors. Identifying these useful combinations is likely to require whole animal screening to account for tumor complexity. Utilizing flies and human cell lines, the data herein suggest that novel drug combinations-low dose bortezomib plus an inhibitor of the PI3K/Akt pathway such as BEZ235—can be effective against a common sub-type of colorectal cancer by promoting ‘induced dependence’. The findings herein also provide a template towards future efforts that emphasize dosing to a specific biological outcome rather than maximum tolerable dose (MTD) (39). One open question is whether each tumor sub-type will require a different therapeutic combination or whether multigenic models such as Drosophila can help identify therapeutics that act across several tumor types.

### REFERENCES

1 Ocana, A., Pandiella, A., Siu, L. L. & Tannock, I. F. Preclinical development of molecular-targeted agents for cancer. Nat Rev Clin Oncol 8, 200-209, (2011).
2 Stratton, M. Exploring the Genomes of Cancer Cells: Progress and Promise. Science 331, 1553-1558, (2011).
3 Hay, M., Rosenthal, J., Thomas, D. & Craighead, J. in BIO CEO & Investor Conference.
4 Caponigro, G. & Sellers, W. R. Advances in the preclinical testing of cancer therapeutic hypotheses. Nature Reviews Drug Discovery 10, 179-187, (2011).
5 Markowitz, S. D. & Bertagnolli, M. M. Molecular origins of cancer: Molecular basis of colorectal cancer. The New England journal of medicine 361, 2449-2460, (2009).
6 Sancho, E., Batlle, E. & Clevers, H. SIGNALING PATHWAYS IN INTESTINAL DEVELOPMENT AND CANCER. Annual Review of Cell and Developmental Biology 20, 695-723, (2004).
7 Leary, R. J. et al. Integrated analysis of homozygous deletions, focal amplifications, and sequence alterations in breast and colorectal cancers. Proceedings of the National Academy of Sciences of the United States of America 105, 16224-16229, (2008).
8 Sjoblom, T. et al. The Consensus Coding Sequences of Human Breast and Colorectal Cancers. Science 314, 268-274, (2006).
9 Wood, L. et al. The Genomic Landscapes of Human Breast and Colorectal Cancers. Science 318, 1108-1113, (2007).
10 Molnar, C. et al. in Human Genetic Diseases (ed Dr. Dijana Plaseska-Karafilska) (InTech, 2011).
11 Hanahan, D. & Weinberg, R. Hallmarks of Cancer: The Next Generation. Cell 144, 646-674, (2011).
12 Fox, D. T. & Spradling, A. C. The Drosophila Hindgut Lacks Constitutively Active Adult Stem Cells but Proliferates in Response to Tissue Damage. Stem Cell 5, 290-297, (2009).
13 Takashima, S., Mkrtchyan, M., Younossi-Hartenstein, A., Merriam, J. R. & Hartenstein, V. The behaviour of Drosophila adult hindgut stem cells is controlled by Wnt and Hh signalling. Nature 454, 651-655, (2008).
14 Brand, A. H. & Perrimon, N. Targeted gene expression as a means of altering cell fates and generating dominant phenotypes. Development 118, 401-415 (1993).
McGuire, S. E., Roman, G. & Davis, R. L. Gene expression systems in Drosophila: a synthesis of time and space. Trends in genetics: TIG 20, 384-391, (2004).
16 Sen, B. & Johnson, F. M. Regulation of SRC family kinases in human cancers. J Signal Transduct 2011, 865819, doi:10.1155/2011/865819 (2011).
17 Kessenbrock, K., Plaks, V. & Werb, Z. Matrix metalloproteinases: regulators of the tumor microenvironment. Cell 141, 52-67, (2010).
18 Bangi, E., Pitsouli, C., Rahme, L. G., Cagan, R. & Apidianakis, Y. Immune response to bacteria induces dissemination of Ras-activated Drosophila hindgut cells. EBO Rep, (2012).
19 Leibowitz, B. J. et al. Uncoupling p53 functions in radiation-induced intestinal damage via PUMA and p21. Mol Cancer Res 9, 616-625, (2011).
20 Milyavsky, M. et al. A distinctive DNA damage response in human hematopoietic stem cells reveals an apoptosis-independent role for p53 in self-renewal. Cell Stem Cell 7, 186-197, (2010).
21 Indran, I. R., Tufo, G., Pervaiz, S. & Brenner, C. Recent advances in apoptosis, mitochondria and drug resistance in cancer cells. Biochim Biophys Acta 1807, 735-745, (2011).
22 Fan, Y. & Bergmann, A. The cleaved-Caspase-3 antibody is a marker of Caspase-9-like DRONC activity in Drosophila. Cell Death Differ 17, 534-539, (2010).
23 Chandeck, C. & Mooi, W. Oncogene-induced cellular senescence. Advances in anatomic pathology 17, 42-48, (2010).
24 Collado, M. et al. Tumour biology: Senescence in premalignant tumours. Nature Cell Biology 436, 642-642, (2005).
25 www.clinicaltrials.gov (2011).
26 Kelley, R. & Venook, A. P. Drug development in advanced colorectal cancer: challenges and opportunities. Current oncology reports 11, 175-185, (2009).
27 Ortega, J., Vigil, C. E. & Chodkiewicz, C. Current progress in targeted therapy for colorectal cancer. Cancer control: journal of the Moffitt Cancer Center 17, 7-15, (2010).
28 Peyton, J. D. et al. in ASCO Annual Meeting. Suppl; Abstr 3066 (J Clin Oncol).
29 Guertin, D. A. & Sabatini, D. M. Defining the role of mTOR in cancer. Cancer Cell 12, 9-22, (2007).
30 Laplante, M. & Sabatini, D. M. mTOR Signaling in Growth Control and Disease. Cell 149, 274-293, (2012).
31 Chen, C. C. et al. FoxOs inhibit mTORC1 and activate Akt by inducing the expression of Sestrin3 and Rictor. Dev Cell 18, 592-604, (2010).
32 Lee, J. H. et al. Sestrin as a feedback inhibitor of TOR that prevents age-related pathologies. Science 327, 1223-1228, (2010).
33 Wu, Y. T. et al. mTOR complex 2 targets Akt for proteasomal degradation via phosphorylation at the hydrophobic motif. J Biol Chem 286, 14190-14198, (2011).
34 Befani, C. D. et al. Bortezomib represses HIF-1alpha protein expression and nuclear accumulation by inhibiting both PI3K/Akt/TOR and MAPK pathways in prostate cancer cells. J Mol Med (Berl) 90, 45-54, (2012).
35 Huang, J. et al. Antitumor activity and drug interactions of proteasome inhibitor Bortezomib in human high-risk myelodysplastic syndrome cells. Int J Hematol 93, 482-493, (2011).
36 Yeramian, A. et al. Inhibition of activated receptor tyrosine kinases by Sunitinib induces growth arrest and sensitizes melanoma cells to Bortezomib by blocking Akt pathway. Int J Cancer 130, 967-978, (2012).
37 Wan, X., Harkavy, B., Shen, N., Grohar, P. & Helman, L. J. Rapamycin induces feedback activation of Akt signaling through an IGF-1R-dependent mechanism. Oncogene 26, 1932-1940, (2007).
38 Samuels, Y. et al. Mutant PIK3CA promotes cell growth and invasion of human cancer cells. Cancer Cell 7, 561-573, (2005).
39 Al-Lazikani, B., Banerji, U. & Workman, P. Combinatorial drug therapy for cancer in the post-genomic era. Nat Biotechnol 30, 679-692, (2012).

What is claimed is:

1. A method for treating a mammalian subject afflicted with a cancer which comprises administering to the mammalian subject (i) a proteasome antagonist and (ii) a PI3K signal transduction pathway antagonist, each of (i) and (ii) in an amount such that when both (i) and (ii) are administered, the administration is effective to treat the mammalian subject.

2. The method of claim 1, wherein the mammalian subject is a human subject.

3. The method of claim 2, wherein the cancer is in the form of a solid tumor.

4. The method of claim 3, wherein the cancer is colon cancer.

5. The method of claim 4, wherein the colon cancer is resistant to treatment.

6. The method of claim 5, wherein the colon cancer is resistant to treatment with a PI3K signal transduction pathway antagonist.

7. The method of claim 3, wherein the PI3K signal transduction pathway antagonist is an organic compound having a molecular weight less than 1000 Daltons, a DNA aptamer, an RNA aptamer, or a polypeptide, which antagonist binds to PI3K, mTor, TORC1, TORC2, AKT, or JNK.

8. The method of claim 7, wherein the PI3K signal transduction pathway antagonist is a DNA aptamer, an RNA aptamer, or a polypeptide.

9. The method of claim 7, wherein the PI3K signal transduction pathway antagonist is an organic compound having a molecular weight less than 1000 Daltons.

10. The method of claim 9, wherein the PI3K signal transduction pathway antagonist binds to PI3K and has the structure: 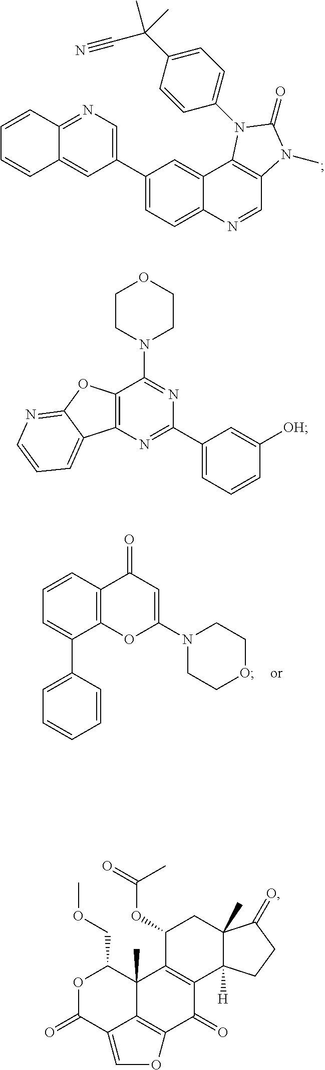 [SMILES: Cn1c(=O)n(-c2ccc(C(C)(C)C#N)cc2)c2c3cc(-c4cnc5ccccc5c4)ccc3ncc21 ; Oc1cccc(-c2nc(N3CCOCC3)c3oc4ncccc4c3n2)c1 ; O=c1cc(N2CCOCC2)oc2c(-c3ccccc3)cccc12 ; COC[C@H]1OC(=O)c2coc3c2[C@@]1(C)C1=C(C3=O)[C@@H]2CCC(=O)[C@@]2(C)C[C@H]1OC(C)=O]
or a pharmaceutically acceptable salt or ester thereof.

11. The method of claim 7, wherein the PI3K signal transduction pathway antagonist is capable of separately binding both PI3K and mTor.

12. The method of claim 9, wherein the PI3K signal transduction pathway antagonist binds to JNK and has the structure: 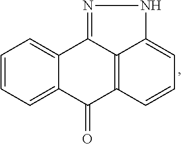 [SMILES: O=C1c2ccccc2-c2n[nH]c3cccc1c23]
or a pharmaceutically acceptable salt or ester thereof.

13. The method of claim 3, wherein the proteasome antagonist is an organic compound having a molecular weight less than 1000 Daltons, a DNA aptamer, an RNA aptamer, or a polypeptide, which antagonist inhibits proteasome function.

14. The method of claim 13, wherein the proteasome antagonist is a DNA aptamer, an RNA aptamer, or a polypeptide.

15. The method of claim 13, wherein the proteasome antagonist is an organic compound having a molecular weight less than 1000 Daltons.

16. The method of claim 15, wherein the proteasome antagonist has the structure: 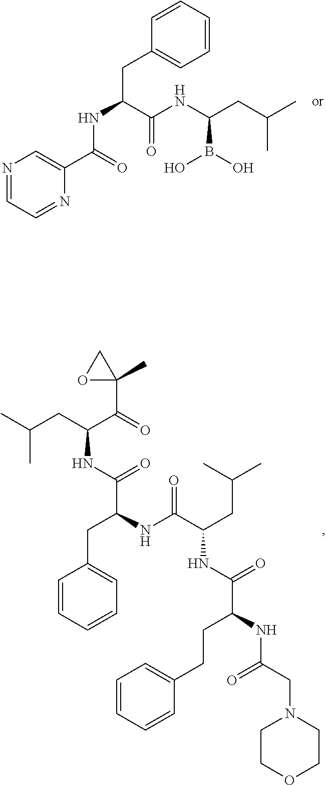
or a pharmaceutically acceptable salt or ester thereof.

17. The method of claim 3, wherein the proteasome antagonist and the PI3K signal transduction pathway antagonist are each an organic compound having a molecular weight less than 1000 Daltons.

18. The method of claim 17, wherein the proteasome antagonist has the structure: 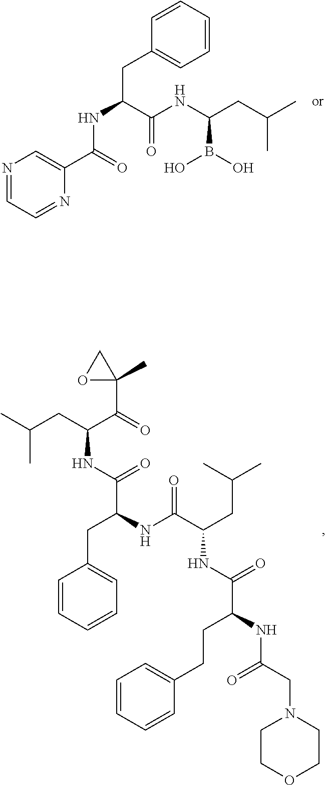
and the PI3K signal transduction pathway antagonist has the structure:

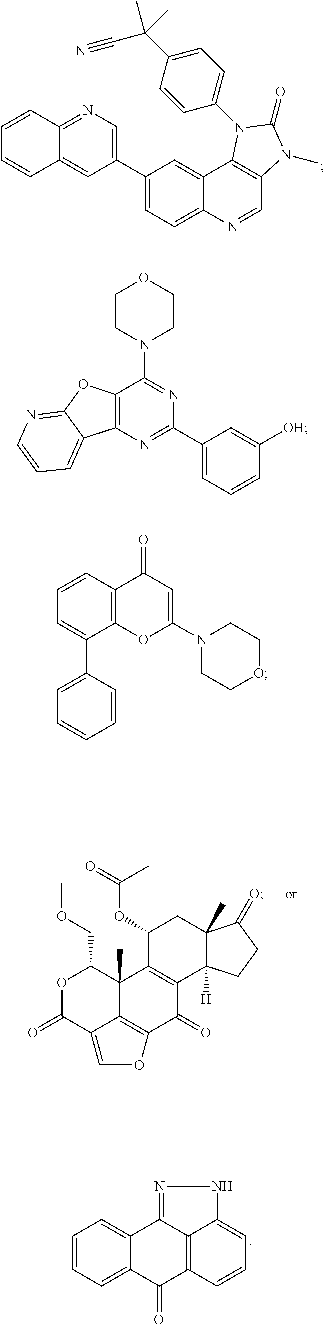 [SMILES: Cn1c(=O)n(-c2ccc(C(C)(C)C#N)cc2)c2c3cc(-c4cnc5ccccc5c4)ccc3ncc21 ; Oc1cccc(-c2nc(N3CCOCC3)c3oc4ncccc4c3n2)c1 ; O=c1cc(N2CCOCC2)oc2c(-c3ccccc3)cccc12 ; COC[C@H]1OC(=O)c2coc3c2[C@@]1(C)C1=C(C3=O)[C@@H]2CCC(=O)[C@@]2(C)C[C@H]1OC(C)=O ; O=C1c2ccccc2-c2n[nH]c3cccc1c23]

19. The method of claim 3, wherein the proteasome antagonist is administered to the mammalian subject before the PI3K signal transduction pathway antagonist, such that the PI3K signal transduction pathway antagonist is administered during at least a portion of the time that the proteasome antagonist is active in the mammalian subject.

20. The method of claim 3, wherein the proteasome antagonist is administered to the mammalian subject concurrently with the PI3K signal transduction pathway antagonist.

21. The method of claim 3, wherein the Receptor Tyrosine Kinase (RTK)/Ras signal transduction pathway and the PI3K signal transduction pathway are misregulated in cells of the cancer compared to cells from tissue of the same type.

22. The method claim 21, wherein the misregulation is selected from the group consisting of:
i) the amount of pAKT is increased and TORC1 activity is decreased in cells of the cancer compared to cells from tissue of the same type;
ii) the Ras signal transduction pathway and the PI3K signal transduction pathway each have a higher level of activation in cells of the cancer compared to cells from tissue of the same type;
iii) Ras and PI3K each have a higher level of activation in cells of the cancer compared to cells from tissue of the same type;
iv) cells of the cancer have at least one activating mutant allele in Ras;
v) cells of the cancer have at least one activating mutant allele in K-Ras;
vi) cells of the cancer have at least one activating mutant allele in N-Ras;
vii) cells of the cancer have at least one activating mutant allele in H-Ras;
viii) cells of the cancer have at least one activating mutant allele in a subunit of PI3K;
ix) cells of the cancer have reduced PTEN function compared to cells from tissue of the same type;
x) cells of the cancer have at least one mutant allele in PTEN that is a deletion mutation, and/or is a mutation that results in the reduced or loss of PTEN protein function in cells of the cancer that express the PTEN mutant protein; and
xi) cells of the cancer have a reduced level of PTEN protein expression compared to cells from tissue of the same type.

23. A method for treating a mammalian subject afflicted with a cancer which comprises administering to the mammalian subject (i) a proteasome antagonist, and (ii) an oligonucleotide which decreases the amount of PI3K, mTor, TORC1, TORC2, AKT, or JNK produced by cells of the cancer, each of (i) and (ii) in an amount that when both (i) and (ii) are administered, the administration is effective to treat the mammalian subject.

24. The method of claim 23, wherein the oligonucleotide is an an antisense oligodeoxynucleotide, a RNA interference inducing compound or a ribozyme that comprises nucleotides in a sequence that is complementary to PI3K, mTor, AKT, or JNK-encoding mRNA.

25. A pharmaceutical composition comprising (i) a proteasome antagonist and (ii) a PI3K signal transduction pathway antagonist or an oligonucleotide which decreases the amount of PI3K, mTor, TORC1, TORC2, AKT, or JNK produced by cells of the cancer, for use in treating a mammalian subject afflicted with a cancer.

26. A method for identifying a cancer patient who will likely benefit from treatment with a PI3K signal transduction pathway antagonist comprising
i) obtaining a biological sample comprising cancer tissue from the cancer patient;
ii) detecting whether the cancer tissue in the biological sample
a) has increased Ras activity and
α) increased PI3K activity, or
β) reduced PTEN expression or activity, or
b) has an increased amount of pAkt and a reduced level of TORC1 activity,
compared to normal tissue of the same type; and
iii) identifying the cancer patient as a cancer patient who will likely benefit from treatment with a PI3K signal transduction pathway antagonist if in step (ii) neither
a) increased Ras activity and
α) increased PI3K activity, or
β) reduced PTEN expression or activity, nor
b) an increased amount of pAkt and a reduced level of TORC1 activity,
is detected in cancer tissue in the biological sample, and identifying the cancer patient as a cancer patient who will not likely benefit from treatment with a PI3K signal transduction pathway antagonist if in step (ii) either
a) increased Ras activity and
α) increased PI3K activity, or
β) reduced PTEN expression or activity, or
b) an increased amount of pAkt and a reduced level of TORC1 activity,
is detected in cancer tissue in the biological sample.

27. The method of claim 26, wherein the cancer patient is selected from the group of cancer patients having colon cancer.

28. A method of treating a cancer patient identified to not likely benefit from treatment with a PI3K signal transduction pathway antagonist in claim 26 comprising administering to the cancer patient (i) a proteasome antagonist and (ii) a PI3K signal transduction pathway antagonist, each of (i) and (ii) in an amount such that when both (i) and (ii) are administered, the administration is effective to treat the cancer patient.
